(19) United States
(12) Patent Application Publication
(10) Pub. No.: US 2022/0054658 A1
(43) Pub. Date: Feb. 24, 2022

(54) CONJUGATES

(71) Applicant: Transfection Holdings Limited, Auckland (NZ)

(72) Inventors: Alan Lawson SEWELL, Auckland (NZ); Ezequiel Silva NIGENDA, Auckland (NZ); Sean Niall O'BYRNE, Auckland (NZ); Ricardo LOPEZ-GONZALEZ, Auckland (NZ); Rodolfo MARQUEZ, Auckland (NZ)

(73) Assignee: Transfection Holdings Limited, Auckland (NZ)

(21) Appl. No.: 17/416,284

(22) PCT Filed: Dec. 18, 2019
(86) PCT No.: PCT/EP2019/086120

§ 371 Date: Jun. 18, 2021

(30) Foreign Application Priority Data
Dec. 18, 2018 (GB) 1820626.8

(51) Int. Cl. A61K 49/00; A61K 47/54; A61K 31/37; A61K 31/4184; A61K 31/7048; A61P 33/02

(52) U.S. Cl. CPC A61K 49/0021; A61K 47/545; A61K 31/37; A61P 33/02; A61K 31/7048; A61K 49/0052; A61K 49/0045; A61K 31/4184

(57) ABSTRACT

The present invention provides a conjugate of formula (I) and its use in methods of treatment, and also methods for delivering an active agent into a cell. The methods may be used to deliver an active agent into a nematode, flatworm, parasite or bacterium. The conjugate of formula (I) is: (formula (I)), wherein -D- is C₁₋₄ alkylene or C₂₋₄ alkenylene, preferably C₂₋₄ alkenylene, where the alkylene or alkenylene is optionally substituted with alkyl or halo; A- is an active agent for delivery; and —R^{A}, —R^{B}, —R^{T1}, —R^{T2}, —R¹, —R², —R³, —X— and -L- are as defined herein.

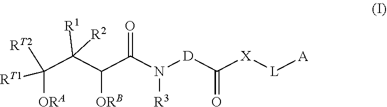

### RELATED APPLICATION

The present application claims the benefit of and priority to GB 1820626.8, filed on 18 Dec. 2018 (18 Dec. 2018), the contents of which are hereby incorporated by reference in their entirety.

### FIELD OF THE INVENTION

The present invention provides conjugates of pantothenic acid or a derivative thereof with an active agent, for delivery of the active agent into a cell or into an organism. The invention also provides the conjugate for use in methods of treatment and methods for the preparation of the conjugate.

### BACKGROUND OF THE INVENTION

Methods for the treatment of parasitic diseases, have recently focused on techniques for improving the delivery of a drug to the location of the parasite within the host, followed by its subsequent uptake into the parasite. Many current methods for the treatment of parasitic diseases rely on the administration to the patient of high levels of drug over sustained periods to ensure that the drug is provided at the target location in sufficient concertation and for sufficient duration to achieve a beneficial effect. The administration of the drug in this way frequently gives rise to serious side effects in the method of treatment.

For this reason it is beneficial to provide a drug delivery strategy that allows lower amounts of the drug to be administered, whilst maintaining the beneficial therapeutic effect. Recent work in this area has looked at all aspects of drug delivery. One line of research has looked at improving the transportation of the active agent within the infected host to the target location. Examples strategies include adaptations of the drug formulation and dosage regimens. For example, the incorporation of an antiparasitic into a nano- or microparticle, an emulsion or a liposome may provide for more targeted delivery of the drug to the pathogen (see, for example, the review of delivery strategies for antiparasitics by Kayser et al. and Date et al.).

Another line of research has focused on the delivery of the drug into the parasite itself, and in particular the transportation of the drug across the parasite cell membrane. The use of many antibiotics is frustrated by the relative inability of the drug to cross cell membranes. To address the problem of delivering the drug into a cell, researchers have looked to connect the drug to a second agent that is known to cross the cell membrane. The second agent may therefore be used to carry the drug into the cell of the parasite.

As an example of this approach for the treatment of malaria and other parasitic diseases, Sparr et al. have described the preparation and use of a conjugate of the antimalarial drug fosmidomycin with an octaarginine peptide. Fosmidomycin is reported to be poorly taken up by T. gondii, M. tuberculosis and P. berghei. This drug was covalently connected to an octaarginine peptide via a short linker, or the drug was prepared as a salt where the counter ion was a labelled octaarginine peptide. The octaarginine peptide has previously been shown to penetrate the cell membrane of red blood cells infected with P. falciparum. The authors showed that the octaarginine peptide could be used to improve the uptake of fosmidomycin into P. falciparum and others, and subsequently improve the antiparasitic effects of the drug.

Landfear has also reported that the uptake of antiparasitic agents into the intracellular environment may be improved if the agents are linked to functionality that is associated with cellular uptake. Thus, Landfear refers to the use of a P2-targeting motif which is known to be a substrate for the P2 transporter for the purpose of increasing selectivity of uptake.

There is a need for further vehicles useful for delivering active agents into parasites. Accordingly, the present inventors have developed a conjugate useful for delivery of active agents into intracellular and extracellular parasites, including nematodes or worms and bacteria, which goes at least some way to meeting this need; and/or at least provides the public with a useful choice.

### SUMMARY OF THE INVENTION

In a general aspect the present invention provides a conjugate comprising a pantothenic acid group or a derivative thereof (“a pantothenic acid group”) for delivery of an active agent into a cell. The pantothenic acid group is covalently linked to the agent, either directly or via a linker.

Thus, the invention allows for the modification of an active agent with a pantothenic acid group to improve or alter the transport properties of the active agent. For example, the pantothenic acid group may improve the transport of the active agent across cellular membranes, thereby increasing the amount of active agent within the intracellular environment.

The conjugates of the invention may be used to deliver an active agent into a cell of a pathogen, such as a bacterial cell or the cells of a parasite, such as a nematode or a worm. The conjugates of the invention may find use in the treatment of a host subject, such as a mammalian subject, who is infected with the pathogen.

The conjugate of the invention may be used to selectively deliver the active agent into a cell that is infected with a pathogen. Thus, the conjugate does not deliver the active agent into uninfected cells.

The conjugate of the invention may also be used to deliver the active agent to a pathogen that is an intracellular or extracellular parasite.

In a first aspect of the invention there is provided a conjugate of formula (I):

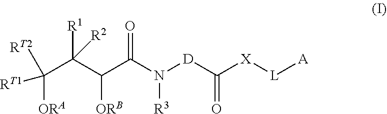

wherein:
—R^{A} and —R^{B} are each independently selected from hydrogen, alkyl, alkenyl, alkynyl, aralkyl, cycloalkylalkyl, alkanoyl and aralkanoyl, such as hydrogen;
or —R^{A} and —R^{B} are together —C(R^{C1})(R^{C2})—, forming a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, such as —R^{C1} and —R^{C2} are alkyl, or —R^{C1} and —R^{C2} are together oxo (═O);
—R^{T1} and —R^{T2} are each independently hydrogen or alkyl, such as hydrogen;
—R¹ and —R² are each independently selected from hydrogen, alkyl, alkenyl, alkynyl, aralkyl and cycloalkylalkyl, such as alkyl;
—R³ is hydrogen or alkyl, such as hydrogen;
D- is C₁₋₄ alkylene or C₂₋₄ alkenylene, such as C₂ alkylene or C₂ alkenylene, where the alkylene or alkenylene is optionally substituted with alkyl or halo;
—X— is a covalent bond, —O—, —S—, —Se—, or —N(R⁴)—, such as —N(R⁴)—, where —R⁴ is hydrogen or alkyl, such as hydrogen;
L- is a linker or a covalent bond; and
A- is an active agent for delivery,
   and salts, solvates and protected forms thereof.

In one embodiment, the compounds of formula (I) is selected from a compound of formula (Ia-I) to (Ia-III):

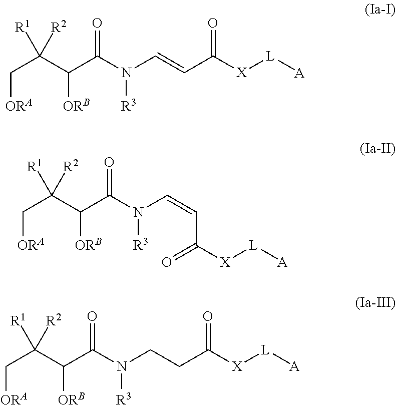

In one embodiment, the compound of formula (I) is of formula (Ib):

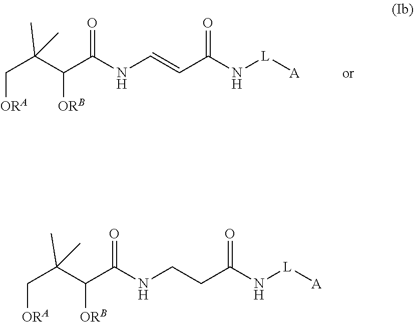

where R^{A}, —R^{B}, -L- and -A are as defined for the compounds of formula (I), and salts, solvates and protected forms thereof.

In one embodiment, the compound of formula (I) is of formula (Ic):

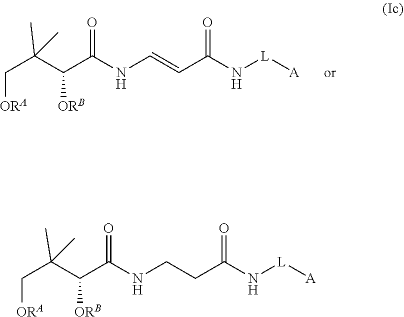

where R^{A}, —R^{B}, -L- and -A are as defined for the compounds of formula (I), and salts, solvates and protected forms thereof.

In one embodiment, the compound is not:

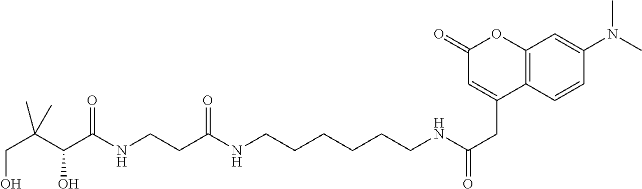 [SMILES: CN(C)c1ccc2c(CC(=O)NCCCCCCNC(=O)CCNC(=O)[C@H](O)C(C)(C)CO)cc(=O)oc2c1]

In a second aspect of the invention there is provided a pharmaceutical composition comprising the conjugate of formula (I) optionally together one or more pharmaceutically acceptable excipients.

In a third aspect of the invention there is provided a conjugate of formula (I) or a pharmaceutical composition comprising the conjugate of formula (I), as described in the first and second aspects of the invention, for use in a method of treatment.

In a fourth aspect of the invention there is provided a conjugate of formula (I) or a pharmaceutical composition comprising the conjugate of formula (I), as described in the first and second aspects of the invention, for use in a method of treating an infection, such as a microbial infection, such as a bacterial infection; or a nematode or flatworm infection; or a parasitic infection.

In various embodiments, the infection may be caused by any one of Haemonchus contortus, Trypanosoma brucei, Theileria annulata, Plasmodium falciparum, Lotmaria passim, Babesia bovis, or Mycobacterium tuberculosis.

In various embodiments, the infection may be caused by worms, kinetidoplasts, apicomplexan, or Mycobacteria.

In a fifth aspect of the invention there is provide the use of conjugate as a tool for drug and protein delivery into Caenorhabditis elegans, which is used as an animal model of human disease.

The invention also provides a method for delivering a conjugate of formula (I) or a pharmaceutical composition comprising the conjugate of formula (I), as described in the first and second aspects of the invention, into a cell, the method comprising the step of contacting a cell with the conjugate of formula (I), or a composition comprising the conjugate.

In one embodiment, the methods of the invention are not methods of treatment of the human or animal body. In on embodiments, the methods of the invention are ex vivo.

In one aspect there is provided compounds for use in the preparation of a compound of formula (I). In one embodiment, the compound is of formula (II), (III), (IV) or (V), which are described in further detail below.

In a further aspect of the invention there is provided a method for the preparation of a compound of formula (I), the method comprising the step of reacting a compound of formula (II), (III) or (IV) with an active agent, thereby to yield a compound of formula (I).

In one embodiment, the method comprises the step of reacting a compound of formula (V) with a compound of formula (II), (III), or (IV), thereby to yield a compound of formula (I).

In yet another aspect of the invention there is provided the use of an active agent for use in a method of treatment, wherein the active agent is in conjugation with a pantothenic acid group. Thus, the active agent may be a conjugate of formula (I).

These and other aspects and embodiments of the invention are discussed in further detail below.

### SUMMARY OF THE FIGURES

FIG. 1 are a pair of fluorescence microscopy images where, left, shows an infected erythrocyte containing two trophozoite stage parasites treated a conjugate of the invention, where the compound is present throughout the cytosol of the parasite. The digestive vacuoles appear as black circles, where there is a lack of compound accumulation; and, right, shows a number of uninfected erythrocytes showing no fluorescence surrounding a trophozoite stage infected cell.

FIG. 2 are a series of microscopy images, including fluorescence microscopy images, of organisms treated with conjugates of the invention, where (a) is a fluorescence microscopy image of an erythrocyte infected with Babesia bovis treated with conjugate 5. Non-infected erythrocytes do not take up the conjugate; (b) is a fluorescence microscopy image of an erythrocyte infected with Theileria parva treated with conjugate 5. The conjugate is seen to accumulate within the parasite; (c) are bright field (left) and fluorescence (right) microscopy images of erythrocytes infected with Plasmodium falciparum treated with conjugate 5. Non-infected erythrocytes do not take up the conjugate; (d) are bright field (left) and fluorescence (right) microscopy images of Trypanomosa brucei treated with conjugate 9 (visible as green spots in the fluorescence images). The DAPI stain is also visible (blue spots in the fluorescence images). Conjugate 5 was also tested (images not shown). Conjugate 5 is taken up by T. brucei and forms small vesicles all over the trypanosome body, whilst not accumulating in either the lysosome or the nucleus, but rather in a vesicle between the flagellar pocket and the lysosome. Conjugate 9 on the other hand, is taken up much more rapidly and in much higher concentrations than compound 5. Compound 9 is also spread throughout the cell, but it does seem to have areas of higher concentration either in the mitochondrion or in the endoplasmic reticulum; (e) is a fluorescence microscopy image of Escherichia coli treated with conjugate 5 showing uptake of the conjugate by the organism; (f) is a fluorescence microscopy image of Enterococcus faecalis treated with conjugate 5 showing uptake of the conjugate by the organism; (g) is a fluorescence microscopy image of Staphylococcus aureus treated with conjugate 5 showing uptake of the conjugate by the organism; (h) are a pair of fluorescence microscopy images of Caenorhabditis elegans treated with conjugates 1 (right image) and 5 (left image). Conjugate 5 is localised with the digestive track of C. elegans. Conjugate 1 is distributed throughout the nematode; and (i) are a pair of fluorescence microscopy images of Haemonchus contortus treated and untreated with conjugate 1. The left image is the control showing the auto-fluorescence of H. contortus. The right image is of H. contortus incubated with conjugate 1.

FIG. 3 shows results of uptake test of delivery vehicles (a) compound 2 and (b) compound 8 in L. passim. Photos were taken from experiments performed at 500 μM for compound 2 and 10 μM for compound 8, and incubation at room temperature for 45 min, size bar: 5 μm.

FIG. 4 shows the results of evaluation of the delivery vehicles and BODIPY 11 on L passim. Bar chart represents RFU's from experiment performed at 1 μM and rt for 45 min incubation. RFU were calculated from images using ImageJ software. (Units×10⁶). The asterisks indicate significant differences (*P<0.03) between the sample and BODIPY control 11 analysed using an independent Student's t-test.

FIG. 5 shows results of uptake test of delivery vehicles 2 and 8 as well as BODIPY unit 11 in honey bee guts. Photos taken from experiments performed at 100 μM and 33° C. for 45 min. incubation, size bar: 100 μm. Photos show brightfield (left image) and FITC filter (right image) images, (a) BODIPY 11; (b) compound 1; (c) compound 2; (d) compound 8.

FIG. 6 shows uptake evaluation of delivery vehicles in RBCs infected with B. bovis. Photos are merged results from brightfield and FITC filter, size bar: 5 μm. a) compound 1; b) compound 3; c) compound 2; d) compound 5; e) BODIPY 11.

FIG. 7 shows uptake evaluation of delivery vehicle (R)-P3 in RBC's infected with B. bovis. Photos are results from experiment performed at 100 μM and 37° C., for 45 min. incubation. Photo: RBC infected by parasites, size bar: 5 μm. (a) Brightfield; (b) DAPI; (c) FITC; (d) merged.

FIG. 8 shows uptake evaluation of ivermectin B1a and compounds 14 and 15 and ivermectin B1a in a mixed culture of wild type nematodes. The images are taken from experiments performed at 5 μM and 50 μM concentrations, 25° C. and 24 h incubation, size bar: 100 μm. (a) ivermectin B1a, 5 μM, Brightfield; (b) compound 14, 5 μM, Brightfield; (c) compound 15, 5 μM, FITC; (d) ivermectin B1a, 50 μM, Brightfield.

FIG. 9 shows uptake evaluation on incubating the bacteria, M. tuberculosis (strain H37Rv), with 50 μg/mL of compounds 1-8, 10 and the control PBS (phosphate-buffered saline). FIG. 9(a) shows a graph of the uptake experiments with the relative fluorescence units (RFUs) of compounds 1-8, 10 and the control PBS after incubation with the bacteria for 45 min. FIG. 9(b) shows two Brightfield images of the M. tuberculosis bacteria for the uptake experiments with compounds 1 (top) and 2 (bottom).

### DETAILED DESCRIPTION OF THE INVENTION

The present inventors have found that pantothenic acid and its derivatives find may be used to modify an active agent to improve the delivery of that agent in vivo.

The conjugate comprises a pantothenic acid group that is covalently linked to an agent, either directly or via a linker group.

Pantothenic acid is utilised in vivo to prepare Coenzyme A (CoA) (see Van der Westhuyzen et al.). The first step in the biosynthesis is the conversion of pantothenic acid to P-Pan, mediated by PanK. This phosphorylated compound is then converted to P-Pan-CMP and then PPC under the action of PPCS.

Recently, analogues of pantothenic acid have been described that are capable of disrupting Coenzyme A biosynthesis. Studies have shown that a range of bacteria are unable to synthesize pantothenate and are therefore dependent on the uptake of pantothenic acid from their environment for the synthesis of CoA. Disrupting CoA biosynthesis is therefore a useful strategy for the treatment of bacterial infections.

An example bioactive derivative of pantothenic acid is CJ-15,801. Structurally, CJ-15,801 differs from pantothenate only in the fact that it has a double bond in the β-alanine moiety. CJ-15,801 is processed in vivo by PanK and PPCS to yield P-CJ-CMP (the CJ-15,801 version of P-Pan-CMP). P-CJ-CMP is a potent tight-binding inhibitor of PPCS. The processing of pantothenic acid is therefore prevented.

Han et at have described a synthetic route to antibiotic CJ-15,801. Several intermediates are protected forms of the antibiotic, such as the carboxylic acid terminus is protected as a benzyl amide or an allyloxycarbonyl group. The protected forms of the antibiotic are not reported to have antibiotic activity and the protecting groups used at the carboxylic acid terminus are not considered to be active agents.

The present inventors have established that active agents may be modified with pantothenic acid and its derivatives to improve the delivery of the active agent, for example to improve the delivery of the active agent into parasites and bacteria. The inventors have established that conjugates containing a pantothenic acid group are rapidly taken up into cells. For example, the inventors have noted the conjugates of the invention are taken up within 45 minutes into S. aureus and E. faecalis cells, amongst others. Thus, the conjugates of the invention find use with gram-positive and gram-negative bacteria.

Additionally, the conjugates of the invention are selective for certain cell types, and, for example, may be preferably taken up into bacterial cells over mammalian cells, or into parasite cells over host cells. In one embodiment, the conjugates of the invention may preferably be taken up into mammalian cells that are infected with a parasite over mammalian cells that are not infected. In another embodiment, the conjugates of the invention may be preferably taken up into cells infected with a parasite over insect cells. The conjugates of the invention are deliverable into prokaryotic cells, such as bacterial cells.

The conjugates of the invention are deliverable into eukaryotic cells, including the cells of eukaryotic microorganisms, such as protists, including for example, Chromalveolata microorganisms, such as Apicomplexa microorganisms, or kinetidoplastids such as Trypanosoma and Lotmaria. The conjugates of the invention are deliverable into a worm, such as C. elegans and H. contortus.

The conjugates of the invention may therefore find use in delivering agents for the treatment of microorganisms that are associated with disease. Thus, the conjugates find use in the treatment of a microorganism infection within a subject, such as a mammalian subject.

Thus, whilst with pantothenic acid and its derivatives are known to have use in vivo within the CoA biosynthesis pathway (either as substrates or antimetabolites), the acid and its derivatives are used in the present invention to modify, such as improve, the transport properties of an active agent.

Clarke et al. have previously described a pantothenic acid analogue for studying the biosynthesis of Coenzyme A (CoA). Here, pantothenic acid was covalently connected to a fluorescent dye via a diaminoalkylene linker. The pantothenic acid analogue was taken up into E. coli cells and was processed within the cell to yield a Coenzyme A analogue.

The pantothenic acid analogue is not used in methods of treatment, and there is no suggestion that the analogue could be delivered into mammalian cells hosting a parasite. Indeed, the authors report that the analogue has very little antibacterial activity, and this is highlighted as an advantage of the analogue.

The work of Clarke et al. is focused solely on studying the metabolic processing of pantothenate compounds within cells. Thus, the pantothenic acid analogue is provided as a substrate for natural product elucidation. There is no suggestion that pantothenic acid or its derivatives should or could be used as delivery vehicles to bring other active agents into a cell.

In one embodiment, a conjugate of the present invention is not compound 1 from Clarke et al. In a further embodiment, the conjugate of the present invention comprises an active agent having biological activity, such as antiparasitic activity, such as antimicrobial activity.

EP 0068485 discloses carbapenem derivatives conjugated to a pantothenic acid group for use as antibiotics. The focus of the patent is to produce these carbapenam derivatives from bacteria, such as Streptomyces sp. OA-6129. The compounds are disclosed to have antimicrobial activity against Comamonas terrigena B-996, a β-lactam-high sensitivity microorganism, however no quantitative data is disclosed. There is no suggestion that pantothenic acid or its derivatives should or could be used as delivery vehicles to bring other active agents into a cell.

The present invention differs from EP 0068485 as the conjugates for use against microbes, such as bacteria, contains at least one unsaturated bond in the in the β-alanine moiety of the pantothenic acid group, forming an enamide. This derivative of the pantothenic acid group is more selective for bacteria than the pantothenic acid group itself (see for example testing on Mycobacterium tuberculosis in the examples).

U.S. Pat. No. 9,108,942 discloses conjugate CLX-SYN-G18-CO1 for use in treating severe pain. The conjugate contains a 2-(2-((2,6-dichlorophenyl)amino)phenyl)acetic acid group as the active agent attached to a pantothenic acid group. The other conjugates disclosed have a wide variety of different groups attached to the active agent. There is no suggestion that pantothenic acid group is preferable to the other list of groups disclosed.

The present invention is novel over CLX-SYN-G18-CO1 as the conjugate of the present invention contains at least one unsaturated bond in the in the β-alanine moiety of the pantothenic acid group, forming an enamide. This derivative of the pantothenic acid group is more selective than the pantothenic acid group itself (see for example testing on Plasmodium falciparum, Trypansoma brucei, Theileria armulata, and Mycobacterium tuberculosis in the examples).

WO 2012 064632 discloses 5-mercapto-1H-indazole-4,7-dione derivatives which can inhibit fatty acid synthase, for use in treating a wide variety of conditions such as bacterial and protozoal infections. Some of the disclosed compounds are conjugated to pantothenic acid groups, amongst a wide variety of other possible groups. There is no biological data provided to support the claim that the compounds have activity against bacteria or protozoa. Protozoa are not necessarily parasites.

The present invention is novel over WO 2012 064632 as the conjugate for use against bacteria or parasites of the present invention contains at least one unsaturated bond in the in the β-alanine moiety of the pantothenic acid group, forming an enamide. This derivative of the pantothenic acid group is more selective for bacteria or parasites than the pantothenic acid group itself (see for example testing on Plasmodium falciparum, Trypansoma brucei, Theileria annulata, and Mycobacterium tuberculosis in the examples).

WO 2012/097454 discloses conjugates for potentiating resistant bacterial cells, comprising an aminoglycoside moiety attached to a pantothenic acid group. The conjugates are reported to lack antibacterial activity on their own but resensitize bacteria towards other antibiotics. There is no suggestion that pantothenic acid or its derivatives should or could be used as delivery vehicles to bring other active agents into a cell.

The present invention is novel over WO 2012/097454 as the conjugate for use against bacteria of the present invention contains at least one unsaturated bond in the in the β-alanine moiety of the pantothenic acid group, forming an enamide. This derivative of the pantothenic acid group is more selective for bacteria than the pantothenic acid group itself (see for example testing on Mycobacterium tuberculosis in the examples).

WO 2019/060634 discloses two compounds comprising a cystamine derivative conjugated to a pantothenic acid group. The compounds are for treating cystamine-sensitive symptoms, syndromes and diseases. This includes infectious diseases such as bacterial and parasitic infections. The example bacteria and parasites stated to cause a cystamine-sensitive infection is Pseudomonas aeruginosa bacteria that cause cystic fibrosis, and Plasmodium falciparum and Plasmodium beghei parasites that cause malaria. Again, there is no suggestion that pantothenic acid or its derivatives should or could be used as delivery vehicles to bring other active agents into a cell.

The present invention is novel over WO 2019/060634 as the active agent in conjugate for use against bacteria and parasites of the present invention contains at least one unsaturated bond in the in the β-alanine moiety of the pantothenic acid group, forming an enamide. This derivative of the pantothenic acid group is more selective for bacteria and parasites than the pantothenic acid group itself (see for example testing on Plasmodium falciparum, Trypansoma brucei, Theileria annulata, and Mycobacterium tuberculosis in the examples).

Meier et al discloses compounds for in vitro and in vivo protein labelling, containing a fluorescent dye moiety conjugated to a pantothenic acid group. These compounds were shown to be incorporated into the CoA pathway of E. coli and attach to the carrier protein Fren. Again, there is no suggestion that pantothenic acid or its derivatives should or could be used as delivery vehicles to bring other active agents into bacteria such as E. coli.

The present invention is novel over Meier et al. as the conjugates for use in a method of delivery into bacteria such as E. coli contain at least one unsaturated bond in the in the β-alanine moiety of the pantothenic acid group, forming an enamide. This derivative of the pantothenic acid group is more selective for bacteria than the pantothenic acid group itself (see for example testing on Mycobacterium tuberculosis in the examples).

Shakya et al. discloses pantothenic acid derivatives as polyketide mimetics. The mimetics attach to actinorhodin acyl carrier protein (actACP) for use in interrogating polyketide synthases (PKSs). There is no suggestion that pantothenic acid or its derivatives should or could be used as delivery vehicles to bring active agents into a cell.

The present invention is novel over Shakya et al. as the conjugates of the present invention contain at least one unsaturated bond in the in the β-alanine moiety of the pantothenic acid group, forming an enamide. Additionally, the moiety attached to the pantothenic group is not a dye, a small drug, a polypeptide, a polynucleotide or a polysaccharide.

Storz et al. discloses compounds that inhibit the PqsD enzyme. Inhibition of PqsD represses the production of 2-heptyl-4-hydroxyquinoline (HHQ) and Pseudomonas quinolone signal (PQS). The molecules HHQ and PQS are involved in the regulation of virulence factor production and biofilm formation of Pseudomonas aeruginosa, a pathogenic gram-negative bacterium. Thus, PqsD is a target for the development of anti-infectives.

The present invention is novel over Storz et al. as the conjugate for use against bacteria of the present invention contains at least one unsaturated bond in the in the β-alanine moiety of the pantothenic acid group, forming an enamide. This derivative of the pantothenic acid group is more selective for bacteria than the pantothenic acid group itself (see for example testing on Mycobacterium tuberculosis in the examples).

### Conjugate

The present invention provides a conjugate of an agent together with a pantothenic acid group. Thus, in one embodiment, the conjugate comprises a group having the structure:

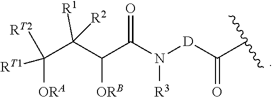

In the present case, references to a pantothenic acid group and its derivatives (“a pantothenic acid group”) is a reference to a group having the structure shown above.

In one aspect of the invention there is provided a conjugate of formula (I):

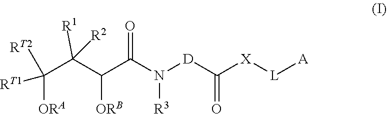

and salts, solvates and protected forms thereof. The substituent groups are discussed in detail below.

### Example Conjugates

In one embodiment, the compound of formula (I) is of formula (Ib):

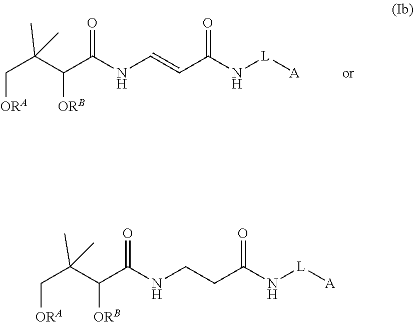

where —R^{A}, —R^{B}, -L- and -A are as defined for the compounds of formula (I), and salts, solvates and protected forms thereof.

In one embodiment, the compound of formula (I) is of formula (Ic):

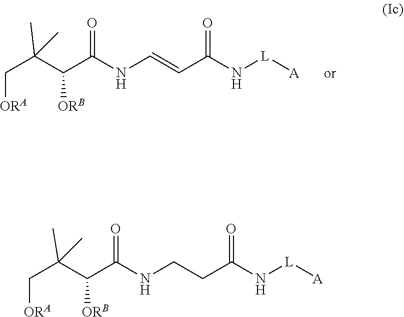

where —R^{A}, —R^{B}, -L- and -A are as defined for the compounds of formula (I), and salts, solvates and protected forms thereof.

In one embodiment, the compound of formula (I) is of formula (Id):

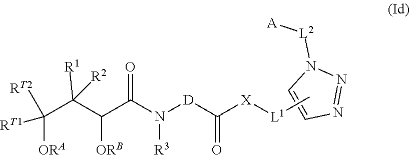

where —R^{T1}, —R^{T2}, —R^{A}, —R^{B}, —R¹, —R², —R³, -D-, and —X— are as defined for the compounds of formula (I);
L¹- is alkylene or heteroalkylene;
L²- is alkylene or heteroalkylene;
   and salts, solvates and protected forms thereof.

In one embodiment, the compound of formula (I) is selected from one of the following formulae:

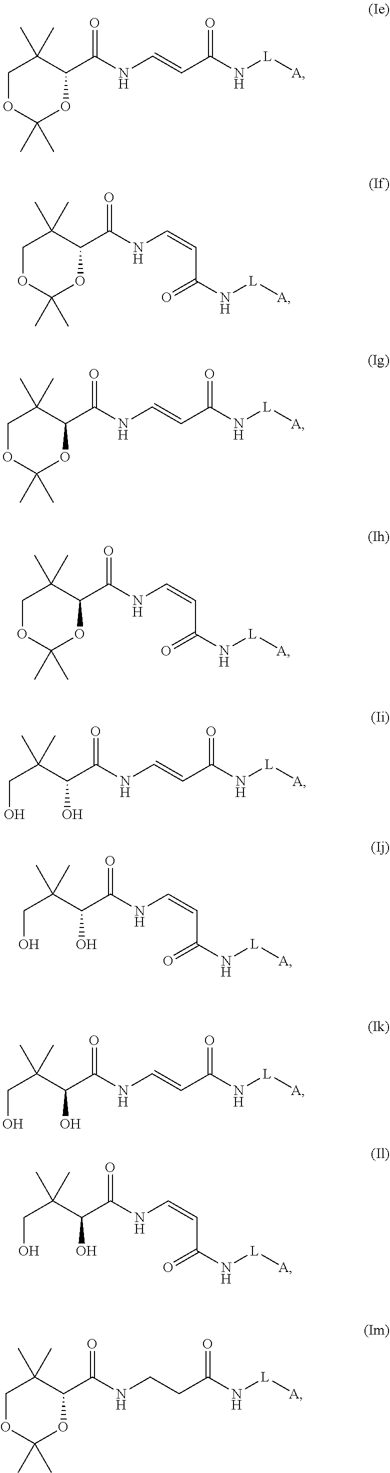

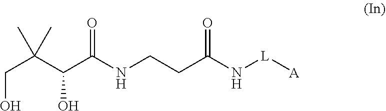

wherein -L- and -A are defined as for the compounds of formula I.

In a preferred embodiment, -L- is a linker as defined for the compounds of formula (Id). In an even more preferred embodiment, -L¹- is a C₂ alkylene, and -L²- is a C₃ alkylene or a C₅ alkylene.

In one embodiment, the compound of formula I is a compound of formula (Ie) or (If). In a preferred embodiment, -L- is a linker as defined for the compounds of formula (Id). In an even more preferred embodiment, -L¹- is a C₂ alkylene, and -L²- is a C₃ alkylene or a C₅ alkylene.

### Intermediate Compounds

In other aspects of the invention there are provided intermediate compounds for use in the preparation of the compounds of formula (I).

Accordingly, there is provided a compound of formula (II).

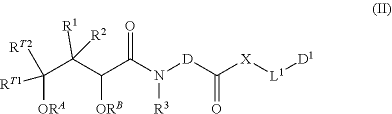

where —R^{T1}, —R^{T2}, —R^{A}, —R^{B}, —R¹, —R², —R³, -D-, and —X— are as defined for the compounds of formula (I);
L¹- is alkylene or heteroalkylene;
D¹ is selected from —OH, —SH, —SeH, —NH₂, —NHR^{N}, —COOH, —COH, —COOR^{D}, —N₃, —C═CH₂, —C═C(H)(Hal) and —C≡CH, where —R^{N} and —R^{D} are each independently alkyl, and Hal is halogen,
and salts, solvates and protected forms thereof.

Also provided is a compound of formula (III):

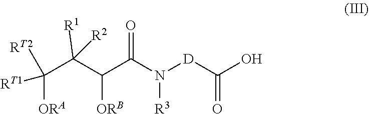

where —R^{T1}, —R^{T2}, —R^{A}, —R^{B}, —R¹, —R², —R³ and -D- are as defined for the compounds of formula (I),
and salts, solvates and protected forms thereof.

The invention also provides a compound of formula (IV):

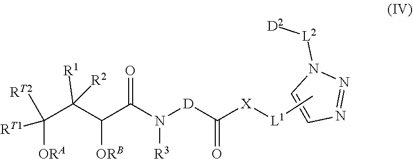

where —R^{T1}, —R^{T2}, —R^{A}, —R^{B}, —R¹, —R², —R³, -D-, and —X— are as defined for the compounds of formula (I);
L¹- is alkylene or heteroalkylene;
L²- is alkylene or heteroalkylene;
D² is selected from —OH, —SH, —SeH, —NH₂, —NHR^{N}, —COOH, —COH, —COOR^{D} and maleimidyl,
and salts, solvates and protected forms thereof.

Also provide, and suitable for use in the invention is a compound of formula (VI:

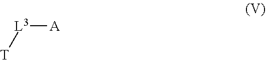

where -A is an active agent;
L³- is a covalent bond, alkylene or heteroalkylene;
T is selected from —OH, —SH, —SeH, —NH₂, —NHR^{N}, —COOH, —COH, —COOR^{D}, —N₃, —C═CH₂ and —C≡CH, where —R^{N} and —R^{D} are each independently selected from alkyl,
and salts, solvates and protected forms thereof.

### Substituent Groups

An alkyl group refers to a monovalent hydrocarbon group. The alkyl group is fully saturated. It may be linear or branched. An alkyl group may be C₁₋₁₀ alkyl, C₁₋₆ alkyl, C₁₋₄ alkyl, C₁₋₂ alkyl or C₁ alkyl (methyl).

An alkylene group refers to a bivalent hydrocarbon group. The alkylene group is fully saturated. It may be linear or branched. An alkylene group may be C₁₋₁₀ alkylene, C₁₋₆ alkylene, C₁₋₄ alkylene, CM alkylene, C₂₋₃ alkylene, C₁₋₂ alkylene, C₂ alkylene (ethylene) or C₁ alkylene (methylene).

An alkenyl group refers to a monovalent hydrocarbon group having one or more carbon-carbon double bonds, such as one double bond. The alkenyl group may be fully or partially unsaturated, such as partially unsaturated. It may be linear or branched. An alkenyl group may be C₂₋₁₀ alkenyl, C₂₋₆ alkenyl, C₂₋₄ alkenyl, C₂₋₃ alkenyl, C₃ alkenyl (allyl) or C₂ alkenyl (vinyl).

An alkenylene group refers to a bivalent hydrocarbon group having one or more carbon-carbon double bonds, such as one double bond. The alkenyl group may be fully or partially unsaturated, such as partially unsaturated. It may be linear or branched. An alkylene group may be C₂₋₁₂ alkenylene, C₂₋₁₀ alkenylene, C₂₋₆ alkenylene, C₄₋₆ alkylene, C₂₋₄ alkenylene, C₂₋₃ alkenylene, C₂ alkenylene, or C₃ alkenylene.

An alkynyl group refers to a monovalent hydrocarbon group having one or more carbon-carbon triple bonds, such as one triple bond. The alkynyl group may be fully or partially unsaturated, such as partially unsaturated. It may be linear or branched. An alkynyl group may be C₂₋₁₀ alkynyl, C₂₋₆ alkynyl, C₂₋₄ alkynyl, C₂₋₃ alkynyl, C₃ alkynyl (propargyl) or C₂ alkynyl.

A cycloalkyl group refers to a monovalent cyclic hydrocarbon group. The cycloalkyl group is fully saturated. A cycloalkyl group may have one ring, or two or more fused rings. The cycloalkyl group may be C₁₋₁₀ cycloalkyl, such as CM cycloalkyl, such C₄₋₆ cycloalkyl, such as C₆ cycloalkyl (cyclohexyl).

A cycloalkylene group refers to a bivalent cyclic hydrocarbon group. The cycloalkylene group is fully saturated. A cycloalkylene group may have one ring, or two or more fused rings. The cycloalkyl group may be C₃₋₁₀ cycloalkylene, such as C₃₋₆ cycloalkylene, such C₄₋₆ cycloalkylene, such as C₆ cycloalkylene (cyclohexylene).

A heteroalkylene group refers to a bivalent hydrocarbon group where one or more carbon atoms is replaced with a heteroatom. The heteroalkylene group is fully saturated. It may be linear or branched.

A heteroalkylene group may be a C₂₋₁₂ heteroalkylene group, such as a C₃₋₁₂ heteroalkylene, such as C₃₋₁₂ heteroalkylene.

A heteroalkylene group may be an alkylene glycol group, such as a polyalkylene glycol group. Examples here include an ethylene glycol group, such as a polyethylene glycol group.

The heteroalkylene group may include one or two heteroatoms, such as one.

The heteroatoms may be selected from —O—, —S— and/or —NH—.

An aryl group refers to a monovalent aromatic group. The aryl group may be a carboaryl group or a heteroaryl group.

A carboaryl group may be C₆₋₁₄ carboaryl, C₆₋₁₀ carboaryl, such as C₆ carboaryl (phenyl) or C₁₀ carboaryl (naphthyl).

A heteroaryl group may be C₅₋₁₀ heteroaryl, such as C₅₋₆ heteroaryl, such as C₅ heteroaryl or C₆ heteroaryl. A heteroaryl group has one or more aromatic ring atoms selected from N, S and O.

Examples of C₅ heteroaryl groups include pyrrolyl and oxazolyl. Examples of C₆ heteroaryl groups include pyridyl and pyrimidinyl.

An aryl group may have one ring, or two or more fused rings. Where a heteroaryl group has two or more rings, each ring may have from 5 to 7 ring atoms, of which 0 to 4 are heteroatoms (with the proviso that at least one ring has one heteroatom).

An arylene group refers to a bivalent aromatic group. The arylene group may be a carboarylene group or a heteroarylene group.

A carboarylene group may be C₆₋₁₄ carboarylene, C₆₋₁₀ carboarylene, such as C₆ carboarylene (phenylene) or C₁₀ carboarylene (naphthylene).

A heteroarylene group may be C₅₋₁₀ heteroarylene, such as C₅₋₆ heteroarylene, such as C₅ heteroaryl or C₆ heteroarylene. A heteroaryl group has one or more aromatic ring atoms selected from N, S and O.

Examples of C₅ heteroarylene groups include triazolylene, pyrrolylene and oxazolylene.

Examples of C₆ heteroarylene groups include pyridylene and pyrimidylene.

An arylene group may have one ring, or two or more fused rings. Where a heteroarylene group has two or more rings, each ring may have from 5 to 7 ring atoms, of which 0 to 4 are heteroatoms (with the proviso that at least one ring has one heteroatom).

A heterocyclene group refers to a bivalent heterocycle. The heterocyclene group is fully saturated.

A heterocyclene may be C₅₋₁₂ heterocyclene, such as C₅₋₇ heterocyclene, such as C₅₋₆ heterocyclene, such as C₆ heterocyclene.

A heterocyclene may have one or two fused rings. Where two rings are present, one or both rings may have a heteroatom.

The heterocyclene may have one or more ring heteroatoms selected from O, S and N (such as NH). The sulfur atom may be oxides, such as SO and SO₂.

A carbon ring atom in a heterocyclene group may have an oxo substituent (═O). In one embodiment, the oxo substituent is provided on a carbon ring atom having a neighbouring nitrogen ring atom, thereby to provide an amido-like group in the heterocycle.

Where the heterocyclene has a nitrogen ring atom, the heterocyclene may be connected via the nitrogen ring atom.

An aralkyl group refers to an alkyl group having one or more, such as one, aryl substituents. The aralkyl is connected via the alkyl group. An example of an aralkyl group is benzyl. The alkyl and aryl groups may each be as defined herein.

A cycloalkylalkyl group refers to an alkyl group having a cycloalkyl substituent. The cycloalkylalkyl group is connected via the alkyl group. The alkyl and cycloalkyl groups may each be as defined herein.

An alkanoyl group refers to an alkyl group where the carbon of the alkyl group that forms the connection is substituted with oxo (═O). An example of an alkanoyl group is acyl (C₂ alkanoyl). The alkanoyl group may be based on an alkyl group as described herein.

An aralkanoyl group refers to an aralkyl group where the carbon of the alkyl group that forms the connection is substituted with oxo (═O). An example of an aralkanoyl group is benzoyl. The aralkanoyl group may be based on an aralkyl group as described herein.

An alkoxy group refers to an alkyl ether, which is connected via the ether oxygen atom. The alkyl group is as defined herein.

An alkenoxy group refers to an alkenyl ether, which is connected via the ether oxygen atom. The alkenyl group is as defined herein. In one embodiment, the ether oxygen is not provided at a carbon atom that also participates in carbon-carbon double bond.

An alkynoxy group refers to an alkynyl ether, which is connected via the ether oxygen atom. The alkynyl group is as defined herein. In one embodiment, the ether oxygen is not provided at a carbon atom that also participates in carbon-carbon triple bond.

An aralkoxy group refers to an aralkyl ether, which is connected via the ether oxygen atom provided on the alkyl of the aralkyl. The aralkyl group is as defined herein.

A cycloalkylalkoxy group refers to a cycloalkylalkyl ether, which is connected via the ether oxygen atom provided on the alkyl of the cycloalkylalkyl. The cycloalkylalkyl group is as defined herein.

—R^{A} and —R^{B}

The groups —R^{A} and —R^{B} are each independently selected from hydrogen, alkyl, alkenyl, alkynyl, aralkyl, cycloalkylalkyl and alkanoyl, or —R^{A} and —R^{B} are together —C(R^{C1})(R^{C2})—, forming a 6-membered ring.

—R^{A} and —R^{B} may together be a group —C(R^{C1})(R^{C2})—. This six-membered ring is formed together with the oxygen to which each of —R^{A} and —R^{B} are attached, and the carbon atoms that are a and p to those oxygen atoms, within the pantoyl moiety:

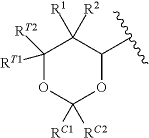

The six-membered ring is a 1,3-dioxane group. The compound may be referred to as an acetal, for example where —R^{C1} and —R^{C2} are alkyl or hydrogen. In another embodiment, —R^{C1} and —R^{C2} together form an oxo (═O) group. Here, the compound may be referred to a cyclic carbonate.

In one embodiment, the —R^{A} and —R^{B} are each independently selected from hydrogen and alkyl, or —R^{A} and —R^{B} are together —C(R^{C1})(R^{C2})—, forming a 6-membered ring.

In one embodiment, —R^{A} and —R^{B} are each independently selected from hydrogen and alkyl, such as hydrogen.

A compound where —R^{A} and —R^{B} are both hydrogen may be formed from a compound where —R^{A} and —R^{B} are together —C(R^{C1})(R^{C2})—. Similarly, compounds where —R^{A} and —R^{B} are not both hydrogen may be formed from compounds where —R^{A} and —R^{B} are both hydrogen.

An alkanoyl group may be a C₁₋₆ alkanoyl group, such as C₁₋₄, such as C₂ alkanoyl (an acyl group). A compound having an alkanoyl group may be formed by reaction of the alcohol with an appropriate acid chloride or anhydride, for example.

Typically, both —R^{A} and —R^{B} are hydrogen or —R^{A} and —R^{B} are together —C(R^{C1})(R^{C2})—, such as —C(Me)₂-.

In one embodiment, —R^{A} is hydrogen.

In one embodiment, —R^{B} is hydrogen.

Stereochemistry The conjugates of the invention are based on pantothenic acid. Thus, the compounds of the invention may also possess the stereochemical configuration of pantothenic acid. This is the (2R)-configuration. Thus, in one embodiment, the conjugate of formula (I) may be:

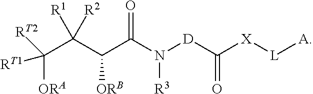

Thus, in one embodiment, a conjugate of the invention has a (2R)-stereochemistry.

In another embodiment, the conjugate of the invention has a (2S)-stereochemistry. Thus, in one embodiment, the conjugate of formula (I) may be:

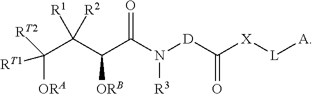

—R^{C1} and —R^{C2}

The group —R^{C1} may be selected from hydrogen, alkyl, alkenyl, alkynyl, aralkyl and cycloalkylalkyl.

The group —R^{C2} may be selected hydrogen, alkyl, alkenyl, alkynyl, aralkyl, cycloalkylalkyl, alkoxy, alkenoxy, alkynoxy, aralkoxy and cycloalkylalkoxy.

Alternatively, —R^{C1} and —R^{C2} are together oxo (═O). This may be referred to as a carbonate protecting group for the diol functionality.

The group —R^{C2} may be an ether group, such as alkoxy. The compound may be referred to as an orthoester.

In one embodiment, —R^{C1} is selected from hydrogen, alkyl and aralkyl.

In one embodiment, —R^{C1} is selected from hydrogen and alkyl.

In one embodiment, —R^{C1} is alkyl, such as methyl.

In one embodiment, —R^{C2} is may be selected from hydrogen, alkyl, alkenyl, alkynyl, aralkyl and cycloalkylalkyl.

In one embodiment, —R^{C2} is selected from hydrogen, alkyl and aralkyl.

In one embodiment, —R^{C2} is selected from hydrogen and alkyl.

In one embodiment, —R^{C2} is alkyl, such as methyl.

In one embodiment, —R^{C1} and —R^{C2} are each alkyl.

In one embodiment, —R^{C1} and —R^{C2} are each methyl.

—R^{T1} and —R^{T2}

The groups —R^{T1} and —R^{T2} are each independently hydrogen or alkyl. The alkyl group may be C₁₋₆ alkyl, such as C₁₋₄ alkyl, such as C₁₋₂ alkyl. The alkyl group may be methyl.

In one embodiment, —R^{T1} is hydrogen and —R^{T2} is hydrogen or alkyl, such as methyl.

In one embodiment, each of —R^{T1} and —R^{T2} is hydrogen.

Pantothenic acid groups having alkyl substituents at the co-position (the terminal position of the pantoyl moiety, also the β-position) are known from Bird et al. (and as discussed by Spry et al.)

### —R^{D}

Each —R^{D} is independently alkyl.

An alkyl group may be a C₁₋₁₂ alkyl group, such as C₁₋₆ alkyl, such as C₁₋₄ alkyl, such as C₁₋₂ alkyl. An alkyl group may be C₁ alkyl (methyl).

In one embodiment, each —R^{D} is methyl.

### —R^{N}

Each —R^{N} is independently alkyl.

An alkyl group may be a C₁₋₁₂ alkyl group, such as C₁₋₆ alkyl, such as C₁₋₄ alkyl, such as C₁₋₂ alkyl. An alkyl group may be C₁ alkyl (methyl).

In one embodiment, each —R^{N} is methyl.

—R¹ and —R²

—R¹ and —R² are each independently selected from hydrogen, alkyl, alkenyl, alkynyl, aralkyl and cycloalkylalkyl.

In one embodiment, —R¹ and —R² are each independently selected from alkyl, alkenyl, aralkyl and cycloalkylalkyl.

In one embodiment, —R¹ and —R² are each alkyl. The groups —R¹ and —R² may both be methyl. One of —R¹ and —R² may be methyl, and the other may be other than methyl.

In one embodiment, —R¹ is the same as —R².

Akinnusi et al. describe geminal derivatives of pantothenamides where the gem-dimethyl substituents of pantothenic acid are replaced with one or two alternative alkyl, alkenyl, aralkyl and cycloalkylalkyl groups.

### —R³

In one embodiment, —R³ is hydrogen.

In one embodiment, —R³ is alkyl, such as methyl.

### -D-

The group -D- is C₁₋₄ alkylene or C₂₋₄ alkenylene, where the alkylene or alkenylene is optionally substituted with alkyl or halo, such as optionally substituted mono- or di-substituted with alkyl or halo, such as optionally substituted mono- or di-substituted with alkyl.

Alternatively, -D- is C₂₋₄ alkenylene where the alkenylene is optionally substituted with alkyl or halo.

In one embodiment, -D- is C₁₋₃ alkylene or C₂₋₃ alkenylene where the alkylene or alkenylene is optionally substituted with alkyl or halo.

Alternatively, -D- is C₂₋₃ alkenylene where the alkenylene is optionally substituted with alkyl or halo.

In one embodiment, -D- is C₂ alkylene or C₂ alkenylene, where the alkylene or alkenylene is optionally substituted with alkyl or halo.

Alternatively, -D- is C₂ alkenylene, where the alkenylene is optionally substituted with alkyl or halo.

The alkylene or alkenylene may be a linear alkylene or linear alkenylene.

In one embodiment, -D- is or —C(R⁵)₂—C(R⁶)₂— or —C(R⁵)═C(R⁶)—, where each —R⁵ is hydrogen or alkyl and each —R⁶ is hydrogen or alkyl.

In one embodiment, -D- is C₂ alkylene or C₂ alkenylene, optionally substituted with alkyl.

Alternatively, -D- is C₂ alkenylene, optionally substituted with alkyl.

Where -D- is C₄ alkenylene, the alkenylene may be a diene.

Preferably, -D- is a C₂₋₃ alkenylene.

Even more preferably, -D- is a C₂ alkenylene.

### Double Bond

In one embodiment, such as in a preferred embodiment, a double bond is present in the conjugate of formula (I).

In one embodiment, the double bond has a trans or cis arrangement.

In one embodiment, the double bond has a trans arrangement. Here, trans refers to the arrangement of the amido groups across the double bond. For example, the compound of formula (Ia-I) has a trans arrangement:

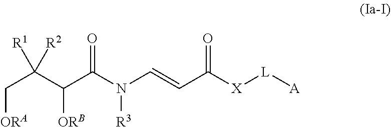

In one embodiment, the double bond has a cis arrangement. Here, cis refers to the arrangement of the amido groups across the double bond. For example, the compound of formula (Ia-II) has a cis arrangement

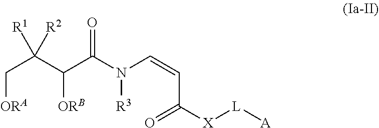

The inventors have found that the geometry of the double bond may influence the selectivity of conjugate to deliver the agent into a particular cell or a particular organism.

Where the group is a diene, the double bonds may both have trans or cis geometry, or one may be trans and the other may be cis.

### —R⁵

Where the pantothenic acid group has a double bond, one group —R⁵ may be present. Where the pantothenic acid group does not have a double bond, two groups —R⁵ may be present. Here, the groups —R⁵ may be the same or different.

In one embodiment, each —R⁵ is hydrogen.

In one embodiment, each —R⁵ is alkyl such as —R⁵ methyl.

### —R⁶

Where the pantothenic acid group has a double bond one group —R⁶ may be present. Where the pantothenic acid group does not have a double bond, two groups —R⁶ may be present. Here, the groups —R⁶ may be the same or different.

In one embodiment, each —R⁶ is hydrogen.

In one embodiment, each —R⁶ is alkyl, such as methyl.

Where a double bond is present, the group —R⁶ may be the same as the group —R⁵. For example, —R⁵ and —R⁶ may both be hydrogen.

Where a double bond is not present, the groups —R⁶ may be the same, and they may be the same as each —R^{%} group as the group —R⁵. For example, each group —R⁵ and each group —R⁶ may be hydrogen.

### —X—

—X— is a covalent bond, —O—, —S—, —Se—, or —N(R⁴)—, such as —N(R⁴)—, where —R⁴ is hydrogen or alkyl, such as hydrogen.

In one embodiment, —X— is a covalent bond, —O—, —S—, or —N(R⁴)—.

In one embodiment, —X— is a covalent bond, —O—, or —N(R⁴)—.

In one embodiment, —X— is a covalent bond or —N(R⁴)—.

Alternatively, —X— may be a covalent bond or —O—.

In one embodiment, —X— is —N(R⁴)—. Exemplary groups include —N(H)— and —N(Me)-.

### —R⁴

In one embodiment, —R⁴ is hydrogen.

In one embodiment, —R⁴ is alkyl, such as methyl.

### -L-

The group -L- may be a covalent bond. Here, the pantothenic acid group together with the group —X— are connected directly to the active agent -A.

Alternatively, the group -L- may be a linker for indirect covalent connection of the pantothenic acid group and the group —X— to the active agent -A.

In one embodiment, the linker -L- is a group *-L³-B-L⁴-G-L^{A}-,
wherein the asterisk indicates the point of attachment to —X—;
-L³- is a covalent bond, alkylene or heteroalkylene;
-B- is a covalent bond, arylene, heterocyclene, or cycloalkylene;
-L⁴- is a covalent bond, alkylene or heteroalkylene; and
-G- is selected from a covalent bond, —O—, —S—, —N(R^{N})—, —C(O)—, —C(O)N(R^{N})—, —C(O)O—, —N(R^{N})C(O)—, —OC(O)—, and a maleimide-derived group, where —R^{N} is hydrogen or alkyl,
-L^{A}- is a covalent bond, alkylene or heteroalkylene,
wherein at least one of -L³-, -B- and -L⁴- is not a covalent bond, and when -B- is a covalent bond, -L⁴- is a covalent bond.

In one embodiment, -L³- and -B- and -L⁴- are not all covalent bonds.

In one embodiment, the linker -L- is a group *-L³-G-L^{A}-,
wherein the asterisk indicates the point of attachment to —X—;
-L³- is a covalent bond, alkylene or heteroalkylene; and
-G- is selected from a covalent bond, —O—, —S—, —N(R^{N})—, —C(O)—, —C(O)N(R^{N})—, —C(O)O—, —N(R^{N})C(O)—, —OC(O)—, and a maleimide-derived group, where —R^{N} is hydrogen or alkyl,
-L^{A}- is a covalent bond, alkylene or heteroalkylene.

In one embodiment, -L³- is a C₃ or C₅ alkylene. In one embodiment, -G- is a maleimide-derived group.

In one embodiment, -L³- is a C₃ or C₅ alkylene, and -G- is a maleimide-derived group.

In one embodiment, the linker -L- is a group *-L³-B-L⁵-G-
wherein the asterisk indicates the point of attachment to —X—;
-L³- is a covalent bond, alkylene or heteroalkylene;
-B- is a covalent bond, arylene, heterocyclene, or cycloalkylene;
-L⁵- is an amide group of formula *—(NR^{N}C(O)-L⁶)-, where the asterisk indicates the point of attachment to -B-, and -L⁶- is alkylene;
G- is a covalent bond, —O—, —S—, —N(R^{N})—, —C(O)—, —C(O)N(R^{N})—, —C(O)O—, —N(R^{N})C(O)—, —OC(O)—, and a maleimide-derived group,
and —R^{N} is hydrogen or alkyl.

In one embodiment, the linker comprises a maleimide-derived group:

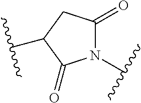 [SMILES: *C1CC(=O)N(*)C1=O]

The maleimide-derived group may be present at the terminal of the linker -L-, for example as a group -G-, for connection to the active agent -A. Thus the connection between the linker and active agent may be formed by the reaction of a thiol group with a maleimide, typically where the active agent possesses thio functionality. For example, the active agent may be a polypeptide having a cysteine residue. Here, the sulfur atom of the active agent is bonded to a carbon ring atom of the maleimide-derived group:

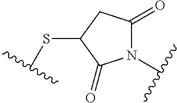 [SMILES: *SC1CC(=O)N(*)C1=O]

Whilst the linkage shown above is formed via a sulfur atom, the maleimide-derived group may also be connected via —O—, —Se—, and —NH—, where such groups may be derived, for example, from the side chain functionality of appropriate amino acid residues (such as Ser, Se-Cys and Lys respectively).

The maleimide-derived group may be a heterocyclene in the linker -L-, for example as a group -B-.

### -L¹-

The group -L¹- is selected from alkylene and heteroalkylene.

A heteroatom present in the heteroalkylene group may not be bonded to the group —X—, particularly when —X— is not a covalent bond.

A heteroatom present in the heteroalkylene group may not be bonded to the group -D¹.

A heteroatom present in the heteroalkylene group may be selected from —O—, —S—, —Se—, or —N(H)—.

In one embodiment, the heteroatom present in the heteroalkylene group may be selected from —O—, —S—, or —N(H)—.

In one embodiment, the heteroatom present in the heteroalkylene group may be selected from —O— or —N(H)—.

In one embodiment, the heteroatom present in the heteroalkylene group is —O—.

In one embodiment, the heteroatom present in the heteroalkylene group is —N(H)—.

In one embodiment, -L¹- is C₁₋₁₂ alkylene, such as C₂₋₆ alkylene, such as C₄₋₆ alkylene, or a C₃₋₅ alkylene.

### -L²-

The group -L²- is selected from alkylene and heteroalkylene.

A heteroatom present in the heteroalkylene group may not be bonded to the nitrogen atom of the triazole.

A heteroatom present in the heteroalkylene group may not be bonded to the group -D².

A heteroatom present in the heteroalkylene group may be selected from —O—, —S—, —Se—, or —N(H)—.

In one embodiment, the heteroatom present in the heteroalkylene group may be selected from —O—, —S—, or —N(H)—.

In one embodiment, the heteroatom present in the heteroalkylene group may be selected from —O— or —N(H)—.

In one embodiment, the heteroatom present in the heteroalkylene group is —O—.

In one embodiment, the heteroatom present in the heteroalkylene group is —N(H)—.

In one embodiment, -L²- is C₁₋₁₂ alkylene, such as C₂₋₆ alkylene, such as C₄₋₆ alkylene.

### -L³-

The group -L³- is selected from a covalent bond, alkylene and heteroalkylene.

A heteroatom present in the heteroalkylene group may not be bonded to the group —X—, particularly when —X— is not a covalent bond.

A heteroatom present in the heteroalkylene group may be selected from —O—, —S—, —Se—, or —N(H)—.

In one embodiment, the heteroatom present in the heteroalkylene group may be selected from —O—, —S—, or —N(H)—.

In one embodiment, the heteroatom present in the heteroalkylene group may be selected from —O— or —N(H)—.

In one embodiment, the heteroatom present in the heteroalkylene group is —O—.

In one embodiment, the heteroatom present in the heteroalkylene group is —N(H)—.

In one embodiment, -L³- is C₁₋₁₂ alkylene, such as C₂₋₆ alkylene, such as C₄₋₆ alkylene.

### -B-

The group -B- is a covalent bond, arylene, heterocyclene, or cycloalkylene.

In one embodiment, -B- is a covalent bond. Here, -L⁴- is also a covalent bond

In one embodiment, -B- is arylene, heterocyclene, or cycloalkylene.

In one embodiment, -B- is arylene, such as carboarylene or heteroarylene.

In one embodiment, -B- is carboarylene, such as phenylene.

In one embodiment, -B- is heteroarylene, such as triazolylene, such as 1,2,3-triazolylene, such as 1,2,3-triazolyl-1,4-ene and 1,2,3-triazolyl-1,5-ene.

In one embodiment, -B- is heterocyclene.

### -L⁴-

The group -L⁴- is a covalent bond, alkylene or heteroalkylene.

A heteroatom present in the heteroalkylene group may not be bonded to the group -G-, particularly when -G- is —O—, —S—, —N(R^{N})—, —C(O)—, —C(O)N(R^{N})—, —C(O)O—, —N(R^{N})C(O)—, and —OC(O)—.

A heteroatom present in the heteroalkylene group may be selected from —O—, —S—, —Se—, or —N(H)—.

In one embodiment, the heteroatom present in the heteroalkylene group may be selected from —O—, —S—, or —N(H)—.

In one embodiment, the heteroatom present in the heteroalkylene group may be selected from —O— or —N(H)—.

In one embodiment, the heteroatom present in the heteroalkylene group is —O—.

In one embodiment, the heteroatom present in the heteroalkylene group is —N(H)—.

### -G-

The group -G- is selected from a covalent bond, —O—, —S—, —N(R^{N})—, —C(O)—, —C(O)N(R^{N})—, —C(O)O—, —N(R^{N})C(O)—, —OC(O)—, and a maleimide-derived group, where —R^{N} is hydrogen or alkyl.

In one embodiment, -G- is a covalent bond.

In one embodiment, -G- is selected from —O—, —S—, —N(R^{N})—, —C(O)—, —C(O)N(R^{N})—, —C(O)O—, —N(R^{N})C(O)—, —OC(O)—, and a maleimide-derived group.

In one embodiment, -G- is selected from —O—, —N(R^{N})—, —C(O)—, —C(O)N(R^{N})—, —C(O)O—, —N(R^{N})C(O)—, —OC(O)—, and a maleimide-derived group.

In one embodiment, -G- is selected from —O—, —C(O)—, —C(O)N(R^{N})—, —C(O)O—, —N(R^{N})C(O)—, —OC(O)—, and a maleimide-derived group.

In one embodiment, -G- is selected from a covalent bond, —C(O)N(R^{N})—, —N(R^{N})C(O)—, and a maleimide-derived group.

In one embodiment, -G- is selected from —C(O)N(R^{N})—, —N(R^{N})C(O)—, and a maleimide-derived group.

In one embodiment, -G- is selected from a covalent bond, —C(O)N(R^{N})—, and a maleimide-derived group.

In one embodiment, -G- is selected from —C(O)N(R^{N})—, and a maleimide-derived group.

In one embodiment, -G- is a maleimide-derived group.

### -L^{A}-

The group -L^{A}- is a covalent bond, alkylene or heteroalkylene.

A heteroatom present in the heteroalkylene group may not be bonded to the group -G-, particularly when -G- is —O—, —S—, —N(R^{N})—, —C(O)—, —C(O)N(R^{N})—, —C(O)O—, —N(R^{N})C(O)—, and —OC(O)—.

A heteroatom present in the heteroalkylene group may be selected from —O—, —S—, —Se—, or —N(H)—.

In one embodiment, the heteroatom present in the heteroalkylene group may be selected from —O—, —S—, or —N(H)—.

In one embodiment, the heteroatom present in the heteroalkylene group may be selected from —O— or —N(H)—.

In one embodiment, the heteroatom present in the heteroalkylene group is —O—.

In one embodiment, the heteroatom present in the heteroalkylene group is —N(H)—.

In one embodiment, -L^{A}- is a covalent bond.

In one embodiment, -L^{A}- is alkylene.

### -L⁵-

The group -L⁵- is an amide group of formula *—(NR^{N}C(O)-L⁶)-, where the asterisk indicates the point of attachment to -B-, and -L⁶- is alkylene.

In one embodiment, -L⁵- is *—(NHC(O)-L⁶)-.

In one embodiment, -L⁶- is C₂₋₁₂ alkylene, such as C₂₋₆ alkylene.

### Triazole

In some embodiments of the invention, a compound contains a triazole group, particularly a 1,2,3-triazole, which may be present in the linker -L-, or in a precursor group to the linker -L-.

Where the 1,2,3-triazole is present, it is 1,4- or 1,5-substituted. In one embodiment, the 1,2,3-triazole is 1,4-substituted.

### -D¹

The group -D¹ is a functional group for forming a covalent bond to an active agent having a functional group, including a modified active agent which is provided with the functional group, for reaction with -D¹.

Thus, -D¹ may be selected from —OH, —SH, —SeH, —NH₂, —NHR^{N}, —COOH, —COH, —COOR^{D}, —N₃, —C═CH₂, —C═C(H)(Hal) and —C≡CH.

In one embodiment, -D¹ may be selected from —OH, —SH, —NH₂, —NHR^{N}, —COOH, —COH, —COOR^{D}, —N₃, —C═CH₂, —C═C(H)(Hal) and —C≡CH.

In one embodiment, -D¹ is selected from OH, —SH, —SeH, —NH₂, and —NHR^{N}.

In one embodiment, -D¹ is selected from —OH, —NH₂, —COOH, —N₃, and —C≡CH.

In one embodiment, -D¹ is selected from —NH₂, —COOH, —N₃, and —C≡CH.

Where -D¹ is —NH₂, this group is suitable for forming amide, carbamate and carbamide bonds.

Where -D¹ is —COOH, this group is suitable for forming amide and ester bonds.

Where -D¹ is —N₃, and —C≡CH, these groups are suitable for forming a linking 1,2,3-triazole group.

Where -D¹ is —C═C(H)(Hal) this group may be suitable for participating in cross-coupling reactions.

### -D²

The group -D² is a functional group for forming a covalent bond to an active agent having a functional group, including a modified active agent which is provided with the functional group, for reaction with -D¹.

Thus, -D² may be selected from —OH, —SH, —SeH, —NH₂, —NHR^{N}, —COOH, —COH, —COOR^{C} and maleimidyl.

In one embodiment, -D² may be selected from —OH, —SH, —NH₂, —NHR^{N}, —COOH, —COH, —COOR^{C} and maleimidyl.

In one embodiment, -D² is selected from —OH, —SH, —SeH, —NH₂, —NHR^{N} and maleimidyl.

In one embodiment, -D² is selected from —OH, —NH₂, —COOH and maleimidyl.

In one embodiment, -D² is maleimidyl.

In one embodiment, -D² is selected from —OH, —NH₂ and —COOH.

### -T

The group -T is a functional group for forming a covalent bond to, for example, a compound of formula (II), (III) or (IV), such as (III). Thus, -T may be reactive with a carboxylic acid group (such as present in (III)) or with a group —NH₂ (such as where -D¹ is —NH₂ in a compound of formula (II) or -D² is —NH₂ in a compound of formula (IV)).

-T may be selected from —OH, —SH, —SeH, —NH₂, —NHR^{N}, —COOH, —COH, —COOR^{D}, —N₃, —C═CH₂ and —C≡CH.

In one embodiment, -T is selected from —OH, —SH, —SeH, —NH₂, and —NHR^{N}. Such groups are suitable for reaction with the carboxylic group present in the compound (III).

In one embodiment, -T is selected from —OH, —NH₂, and —NHR^{N}.

In one embodiment, -T is —NH₂ or —NHR^{N}.

In one embodiment, -T is selected from —COOH, —COH, and —COOR^{D}. Such groups are suitable for reaction with an amino group present in compound (II) or (IV).

In one embodiment, -T is —COOH.

In one embodiment, -T is —N₃ or —C≡CH. Such groups are suitable for reaction with —C≡CH or —N₃ respectively, where such are present in compound (II) or (IV).

### A-

The conjugate contains an active agent for delivery into a cell. The group -A is a radical of the active agent, which may be formally (and not necessarily in practice) derived by removal of a hydrogen radical from the active agent.

The active agent may be a biologically active agent, such as an agent for use in a method of treatment. The active agent, such as when the active agent is a small organic molecule, may have a molecular weight of 1,000 Da or less, such as 500 Da or less, it may be referred to as a small drug. Optionally, the active agent has a molecular weight of 150 Da or more, such as 175 Da or more, such as 200 Da or more.

The active agent may be biologically active even when not attached to the structure shown below:

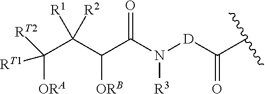

where —R^{A}, —R^{B}, —R^{T1}, —R^{T2}—R¹—R², —R³, and -D- are as defined above (that is, an active agent not in conjugation with a pantothenic acid group).

The active agent may be a compound suitable for use in the treatment of a microbial, such as a bacterial, infection.

The active agent may be a compound suitable for use in the treatment of a parasitic infection.

The active agent may be a compound suitable for treating a nematode or worm, such as flatworm, infection.

The active agent may be a compound suitable for treating a Mycobacterium infection, an Escherichia infection, a Staphylococcus infection or an Enterococcus infection.

The active agent may be a compound suitable for treating a Plasmodium infection, a Trypanosoma infection, a Theileria infection, or a Babesia infection, and additionally or alternatively, a Phytophthora infection, a Crithidia infection, or a Lotmaria infection.

The active agent may be a compound suitable for treatment a Caenorhabditis infection or a Haemonchus infection.

In the conjugates of the invention, it is expected that the active agent is not pantothenic acid or its derivatives. Thus, in one embodiment, -A is not a pantothenic acid group, and thus -A does not have the structure shown below:

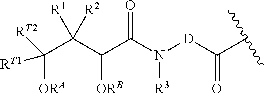

where —R^{A}, —R^{B}, —R^{T1}, —R^{T2}—R¹—R², —R³, and -D- are as defined above.

Thus, in one embodiment, the conjugate is not a dimer of pantothenic acid groups.

The group -A may be a dye, such as an organic dye.

In one embodiment, the dye is a fluorescent dye.

In one embodiment, -A is not a fluorescent dye.

The active agent may be or comprise a polypeptide, such as a protein.

The active agent may be or comprise a polynucleotide.

The active agent may be or comprise a polysaccharide.

Additionally, the polysaccharide may be a disaccharide or a trisaccharide, or a polysaccharide having three or more saccharide units. In one embodiment, the active agent may not be a disaccharide.

The conjugate of the present invention is for use in the delivery of an agent to a desired location, such as within a cell.

The agent is not particularly limited, and may be any agent whose presence at a particular location is considered desirable.

The agent may be an active agent for use in a method of treatment or a method of diagnosis.

Generally, the active agent will have a functional group for forming a covalent connection with the linker. Thus, the active agent may have one or more groups selected from —OH, —SH, —NH₂, —NHR^{N}, —COOH, —COH, —COOR^{C}, —N₃, —C═CH₂, —C≡CH and maleimidyl.

For example, thiol-containing (—SH) active agents, such as cysteine-containing polypeptides, may form a connection to a maleimide group on a linker precursor, such as a compound (III) or (IV).

The active agent may be modified to incorporate a particular functional group for forming a covalent connection to the pantothenic acid group.

### Activity

The conjugates of the invention may have activity against pathogens, such as bacteria and nematodes. Here, the conjugate of the invention is typically provided with an active agent which possess the requisite biological activity.

The compounds of the invention may find use in methods of treatment, such as described in further detail below.

In one embodiment, the conjugate may reduce parasitemia by at least 20%, at least 40%, at least 50%, at least 70, at least 80, or at least 90% as compared with an untreated population or compared with a population of cells treated with the active agent alone (that is, an active agent not in conjugation with a pantothenic acid group).

The parasitemia may be determined at, for example, 48 h or 72 h from initial treatment of the parasite population.

The parasite may be a Plasmodium parasite, such as P. falciparum, or a Theileria parasite, such as T. annulata.

Additionally or alternatively, the parasite may be a parasite as described below.

The parasite may be an apicomplexan or a kinetidoplastid.

The parasite may be a Theileria parasite, such as T. annulata and T. parva, or a Phytophthora parasite, such as P. cinnamomi and P. agathidicida, or a Babesia parasite, such as B. bovis, or a Crithidia parasite, such as C. bombi, or a Lotmaria parasite, such as L. passim, or a Toxoplasma parasite, such as T. gondii.

The parasite may also be a Plasmodium parasite, such as P. vivax, P. ovate, P. malaria and P. knowlesi, or a Trypanosoma parasite, such as T. brucei.

The parasite may be a Plasmodium parasite, such as P. falciparum.

A conjugate of the invention may be a compound having antimicrobial or anthelmintic activity.

In one embodiment, the conjugate may have an antimicrobial activity as measured by MIC of at most 150 μM, at most 100 μM, at most 50 μM, at most 25 μM, at most 10 μM, or at most μM.

Additionally or alternatively, the conjugate may be a compound having antibacterial activity, such as against a bacterium as described below.

The bacterium may be a Mycobacteria bacterium, such as M. tuberculosis.

The bacterium may be an Enterococcus bacterium, such as E. faecalis.

The bacterium may be an Escherichia bacterium, such as E. coli, or a Staphylococcus bacterium, such as S. aureus.

The antimicrobial and anthelmintic activity may be determined using an assay as described herein.

In one embodiment, the conjugate may have an anthelmintic activity as measured by LC₅₀ of at most 10.0 μg/mL, at most 5.0 μg/mL, at most 2.0 μg/mL, at most 1.0 μg/mL, at most 0.5 μg/mL, or at most 0.1 μg/mL.

Additionally, the helminth may be a nematode or worm, such as a flatworm. The nematode or worm may be a Caenorhabditis nematode, such as C. elegans, or a Haemonchus nematode, such as H. contortus, or a Schistosoma flatworm, such as S. haematobium.

The active agent may have biological activity, and when the active agent is used alone (that is, an active agent not in conjugation with a pantothenic acid group) it may have the antiparasitic, antimicrobial or anthelmintic activities described above in relation to the conjugate. However, it is a feature of the invention that the conjugate is provided to enhance the biological activity of the active agent by ensuring that the active agent can be delivered into the cells of the target organism. Thus, in embodiments of the invention the conjugate containing the active agent has an improved activity compared with the active agent used alone.

The conjugate of the invention may have a low toxicity. The toxicity of the conjugate, as measured against the percentage viability of a human embryonic kidney cell line treated with a conjugate, for example, may be 40% or less, such as 30% or less, such as 20% or less, such as 10% or less. The compounds may be used at a concentration of 100 μM.

### Salts, Solvates and Other Forms

Examples of salts of the compounds of the invention, such as the conjugate of formula (I), include all pharmaceutically acceptable salts, such as, without limitation, acid addition salts of strong mineral acids such as HCl and HBr salts and addition salts of strong organic acids such as a methanesulfonic acid salt. Further examples of salts include sulphates and acetates such as trifluoroacetate or trichloroacetate.

A compound of formula (I) can also be formulated as prodrug. Prodrugs can include an antibacterial compound herein described in which one or more amino groups are protected with a group which can be cleaved in vivo, to liberate the biologically active compound. In one embodiment the prodrug is an “amine prodrug”. Examples of amine prodrugs include sulphomethyl, as described in e.g., Bergen et al, Antimicrob. Agents and Chemotherapy, 2006, 50, 1953 or HSO₃-FMOC, as described in e.g. Schechter et a/, J. Med Chem 2002, 45(19) 4264, and salts thereof. Further examples of amine prodrugs are given by Krise and Oliyai in Biotechnology; Pharmaceutical Aspects, 2007, 5(2), 101-131.

In one embodiment a compound of formula (I) is provided as a prodrug.

A reference to a compound of the present disclosure is also a reference to a solvate of that compound. Examples of solvates include hydrates.

A compound of the present disclosure includes a compound where an atom is replaced by a naturally occurring or non-naturally occurring isotope. In one embodiment the isotope is a stable isotope. Thus a compound described here includes, for example deuterium containing compounds and the like. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r- forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₆ alkyl includes n-propyl and iso-propyl; butyl includes n-, iso-, sec-, and tert-butyl; methoxyphenyl includes ortho-, meta-, and para-methoxyphenyl).

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

The compounds described herein may be provided in a protected form. Thus, one or more functional groups within compound may be provided with a protecting group to prevent their unintended reaction, for example during synthesis or storage.

It may be convenient or desirable to prepare, purify, and/or handle a compound in a chemically protected form. The term “chemically protected form,” as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, “Protective Groups in Organic Synthesis” (T. Green and P. Wuts; 3rd Edition; John Wiley and Sons, 1999).

For example, an amine group may be protected as an amide or a urethane, for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH—Fmoc), as a 6-nitroveratryloxy amide (—NH—Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH—Psec); or, in suitable cases, as an N-oxide (>NO.).

A carboxylic acid group may be protected as an ester for example, as: a C₁₋₇ alkyl ester (e.g. a methyl ester; a f-butyl ester); a C₁₋₇haloalkyl ester (e.g. a C₁₋₇ trihaloalkyl ester); a triC₁₋₇ alkylsilyl-C₁₋₇ alkyl ester; or a C₅₋₂₀ aryl-C₁₋₇ alkyl ester (e.g. a benzyl ester; a nitrobenzyl ester); or as an amide, for example, as a methyl amide.

A hydroxyl group may be protected as an ether (—OR) or an ester (—OC(═O)R), for example, as: a f-butyl ether; a benzyl, benzhydryl (diphenylmethyl), or trityl (triphenylmethyl) ether; a trimethylsilyl or f-butyldimethylsilyl ether; or an acetyl ester (—OC(═O)CH₃, —OAc).

The compounds for use in the invention may have 1,3-diol functionality, where each of —R^{A} and —R^{B} is hydrogen. Each of the hydroxyl groups may be independently protected as ether or ester forms. In the present invention the diol may also be protected as an acetal. Thus, —R^{A} and —R^{B} are together —C(R^{C1})(R^{C2})—, forming a 6-membered ring, where each —R^{C1} and —R^{C2} is each independently selected from hydrogen, alkyl, alkenyl, alkynyl, aralkyl and cycloalkylalkyl.

An aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

One aspect of the present invention pertains to compounds in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Methods for Preparing Conjugates

The invention also provides methods for the preparation of conjugates of the invention, including conjugates of formula (I).

The worked examples in the present describe methods for the preparation of conjugates, and these methods may be adapted for the preparation of other conjugates of formula (I).

Generally, the methods of the invention take a pantothenic acid group and react that group with an active agent to form a conjugate. Where the conjugate contains a linker, the linker may be attached to either the pantothenic acid group or the active agent and the linker may then form the connection between the two. For example, in some of the methods of the invention the pantothenic acid group is provided with a linker terminating in a maleimide group. This maleimide group is reacted with an active agent, such as a polypeptide, having thiol functionality to form the conjugate.

In other methods, both the pantothenic acid group and the active agent may contain part of a linker, and the reaction between the pantothenic acid group and the active agent may form the completed linked group. For example, in some of the methods of the invention the pantothenic acid group is provided with a part of a linker terminating in an acetylene group. The active agent is provided with a part of a linker terminating in an azide group. The acetylene group is reacted with the azide group in a click-chemistry type reaction to form an imidazole, thereby generating the linker between the pantothenic acid group and the active agent.

The preparation of pantothenic acid derivatives is described in the art. These methods may be adapted for use in the present invention.

The methods of the invention may make use of intermediate compounds of formula (II), (III) and (IV), and optionally the compound of formula (V) also.

In one aspect, there is provided a method for preparing a compound of formula (I), the method involving the step of reacting a compound of formula (II), (III) or (IV) with an active agent, thereby to yield a compound of formula (I).

Here the active agent has functionality for reaction with a compound of formula (II), (III) or (IV). For example, the compounds of formula (III) is a carboxylic acid at its terminal. This group may be reacted with hydroxyl, thiol or amino functionality present on the active agent to form a conjugate having ester, thioester or amido functionality.

In one aspect of the invention there is provided a method of preparing a conjugate of the invention, the method comprising the step of reacting a compound of formula (II) with a compound of formula (V).

Here the active agent (V) and the and the pantothenic acid group (II) are both provided with a part of the linker, and the linker parts are provided with suitable functionality for reaction together, thereby to form the complete linker.

The compound of formula (II) is:

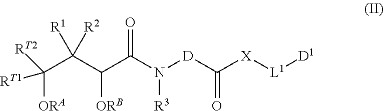

where —R^{T1}, —R^{T2}, —R^{A}, —R^{B}, —R¹, —R², —R³, -D-, and —X— are as defined for the compounds of formula (I);
-L¹- is alkylene or heteroalkylene;
-D¹ is selected from —OH, —SH, —SeH, —NH₂, —NHR^{N}, —COOH, —COH, —COOR^{D}, —N₃, —C═CH₂, —C═C(H)(Hal) and —C═CH, where —R^{N} and —R^{D} are each independently alkyl, and Hal is halogen,
and salts, solvates and protected forms thereof.

Accordingly, the compounds of formula (II) are suitable for preparing conjugates of formula (I) where -L- contains an alkylene or heteroalkylene group.

The compound of formula (II) may be reacted directly with an active agent, as described above, where the active agent is provided with suitable functionality for reaction with the group -D¹.

Alternatively, the compound of formula (II) may be reacted with an active agent that is optionally provided with a linker part, such as the compound of formula (V).

The compound of formula (V) is:

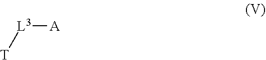

where -A is an active agent;
-L³- is a covalent bond, alkylene or heteroalkylene;
-T is selected from —OH, —SH, —SeH, —NH₂, —NHR^{N}, —COOH, —COH, —COOR^{D}, —N₃, —C═CH₂ and —C≡CH, where —R^{N} and —R^{D} are each independently selected from alkyl,
and salts, solvates and protected forms thereof.

Thus, the group -T is provided for reaction with the group -D¹ of the compound of formula (II).

The reaction of compound (II) with compound (V) provides a conjugate of formula (I) having a linker containing an alkylene or heteroalkylene group that is connected to the active agent, or to an alkylene or heteroalkylene group via the group formed from the reaction of -T and -D¹.

Alternatively, the compound of formula (III) may be reacted with an active agent that is optionally provided with a linker part, such as the compound of formula (V).

The compound of formula (III) is:

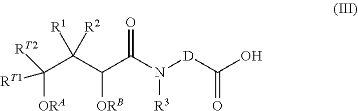

where —R^{T1}, —R^{T2}, —R^{A}, —R^{B}, —R¹, —R², —R³ and -D- are as defined for the compounds of formula (I),
and salts, solvates and protected forms thereof.

In the compound of formula (V), the group -T is a group suitable for reaction with a carboxylic acid. For example, -T is —OH, —SH, —NH₂, or —NHR^{N}, such as —NH₂, or —NHR^{N} such as —NH₂.

The reaction of compound (III) with compound (V) provides a conjugate of formula (I) having a pantothenic acid group that is connected to the active agent, or a pantothenic acid group that is connected to an alkylene or heteroalkylene group via the group formed from the reaction of -T and the carboxylic group.

The compound of formula (III) may be used to prepare a compound of formula (II).

Alternatively, the compound of formula (IV) may be reacted with an active agent that is optionally provided with a linker part, such as the compound of formula (V).

The compound of formula (IV) is:

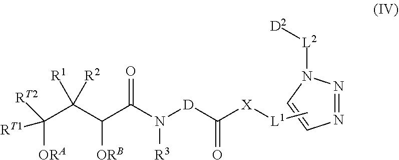

where —R^{T1}, —R^{T2}, —R^{A}, —R^{B}, —R¹, —R², —R³, -D-, and —X— are as defined for the compounds of formula (I);
-L¹- is alkylene or heteroalkylene;
-L²- is alkylene or heteroalkylene;
-D² is selected from —OH, —SH, —SeH, —NH₂, —NHR^{N}, —COOH, —COH, —COOR^{D} and maleimidyl,
and salts, solvates and protected forms thereof.

The methods described above are based on the reaction of common functionality to form standard bonds. Thus, the reaction of amino and carboxyl functionality is anticipated, providing an amide bond. Similarly, the reaction of acetylene and azide functionality is anticipated, providing a triazole linking group. The reaction conditions necessary to achieve this bond formation is well known to the skilled person, and exemplary conditions are provided in the worked examples of the present case.

### Methods of Treatment

The compounds of formula (I), or a pharmaceutical formulation containing this compound, are suitable for use in methods of treatment and prophylaxis. The compounds may be administered to a subject in need thereof.

The conjugates of the invention may be used in methods for the treatment of parasitic, such as microbial, including bacterial infections, protozoan infections, or helminthic infections, such as nematode or worm infection, such as flatworm infection.

The conjugates of formula (I) are for use in a method of treatment of the human, or animal body by therapy. In some aspects of the invention, a compound of formula (I) may be administered to a mammalian subject, such as a human, to treat a microbial infection, such as a bacterial infection.

Another aspect of the present invention pertains to use of a compound of formula (I) in the manufacture of a medicament for use in treatment. In one embodiment, the medicament comprises a conjugate of formula (I). In one embodiment, the medicament is for use in the treatment of a microbial infection, such as a bacterial infection.

In one embodiment, the conjugate is suitable for use as an anthelmintic. Thus, the conjugate may be used to treat a helminth infection, such as a nematode or worm infection, such as schistosomiasis.

Additionally or alternatively, the conjugate may be used to treat a Haemonchus infection, such as a H. contortus infection. The conjugate may therefore be used to treat haemonchosis. The subject for treatment may be a mammal, such as a sheep or goat.

Additionally or alternatively, the conjugate may be used to treat a Schistosoma infection, such as a S. haematobium infection. The conjugate may therefore be used to treat schistosomiasis. The subject for treatment may be a mammal, such as a human.

In one embodiment, the conjugate used to treat a worm infection has the structure Ie or II.

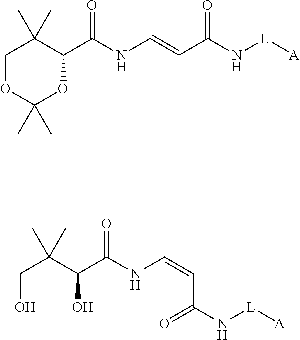

In one embodiment, L is a linker as defined for the compounds of formula (Id). In one embodiment, -L²- is a C₃ alkylene. In a preferred embodiment, -L¹- is a C₂ alkylene and -L²- is a C₃ alkylene.

In one embodiment, the linker -L- is a group *-L³-G-L^{A}-,
wherein the asterisk indicates the point of attachment to —X—;
-L³- is an alkylene; and
-G- is a maleimide-derived group; and
-L^{A}- is a covalent bond.

In a preferred embodiment, -L³- is a C₃ alkylene.

In one embodiment, the conjugate is for use in the treatment of an Escherichia infection, such as an E. Coli infection, or a Staphylococcus infection, such as an S. Aureus infection.

Additionally or alternatively, the, the conjugate is for use in the treatment of an Enterococcus infection, such as an E. faecalis infection.

In one embodiment, the conjugate is for use in the treatment of an Apicomplexan infection.

For example, the conjugate may be used to treat a Babeosia infection, for example B. bovis. The conjugate may therefore be used to treat babesiosis. The subject for treatment here may be a mammal, such as a bovine subject.

In one embodiment, the conjugate used to treat a Babeosia infection has the structure Ie or Ii.

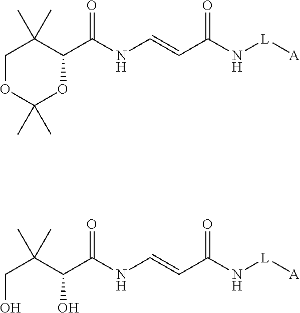

In one embodiment, L is a linker as defined for the compounds of formula (Id). In one embodiment, -L²- is a C₃ alkylene. In a preferred embodiment, -L¹- is a C₂ alkylene and -L²- is a C₃ alkylene.

In one embodiment, the linker -L- is a group *-L³-G-L^{A}-,
wherein the asterisk indicates the point of attachment to —X—;
-L³- is an alkylene; and
-G- is a maleimide-derived group; and
-L^{A}- is a covalent bond.

In a preferred embodiment, -L³- is a C₅ alkylene.

The conjugate may be used to treat a Theileria infection, such as T. annulata and T. parva. The conjugate may therefore be used to treat tropical theileriosis and East Coast fever. The subject for treatment may be a mammal, such as a bovine or an ovine subject.

The conjugate may be used to treat a Plasmodium infection, such as P. falciparum, P. vivax, P. ovate, P. malaria and P. knowlesi. The conjugate may therefore be used to treat malaria. The subject for treatment may be a mammal, such as a human.

Additionally or alternatively, the conjugate may be used to treat a Phytophthora infection, such as P. cinnamomi and P. agathidicida. The conjugate may therefore be used to treat kauri dieback. The subject for treatment may be a plant, such as a tree.

The conjugate may be used to treat a Toxoplasma infection, such as T. gondii. The conjugate may therefore be used to treat Toxoplasmosis. The subject for treatment may be a mammal, such as a human.

The conjugate may be used to treat a kinetoplastid infection, such as a Trypanosoma infection such as a Trypanosoma brucei infection. The conjugate may therefore be used to treat sleeping sickness.

The conjugate may be used to treat a Lotmaria infection, such as a Lotmaria passim infection. Additionally or alternatively, the conjugate may be used to treat a Crithidia infection, such as C. bombi. The subject may therefore be a bee, such as a bumblebee.

In one embodiment, the conjugate used to treat a Lotmaria infection has the structure If.

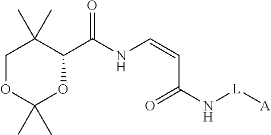

The conjugate may be used to treat a Mycobacteria infection, such as a Mycobacteria tuberculosis infection. The conjugate may therefore be used to treat tuberculosis.

In one embodiment, the conjugate used to treat a Mycobacteria infection has the structure If.

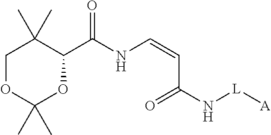

A conjugate of formula (I) may be administered in conjunction with a second active agent. Administration may be simultaneous, separate or sequential.

The methods and manner of administration will depend on the pharmacokinetics of the compound of conjugate (I) and the second active agent.

By “simultaneous” administration, it is meant that a compound of formula (I) and a second active agent are administered to a subject in a single dose by the same route of administration.

By “separate” administration, it is meant that a compound of formula (I) and a second active agent are administered to a subject by two different routes of administration which occur at the same time. This may occur for example where one agent is administered by infusion and the other is given orally during the course of the infusion.

By “sequential” it is meant that the two agents are administered at different points in time, provided that the activity of the first administered agent is present and ongoing in the subject at the time the second agent is administered.

### Methods of Delivery

The conjugates of the invention may be used in methods of delivery, such as methods for delivery of an active agent into an organism, including into the cell of an organism.

The method of the invention includes the step of exposing a conjugate of the invention, such as the conjugate of formula (I), to an organism, and permitting the conjugate to pass into the organism.

The organism may be a helminth, such as a nematode or worm. The nematode or worm may ingest the conjugate. The nematode or worm may be a Caenorhabditis nematode, such a Caenorhabditis elegans nematode, or a Haemonchus nematode, such as Haemonchus contortus nematode, or a Schistosoma flatworm, such as Schistosoma haematobium flatworm.

In a more preferred embodiment, the organism is a nematode or worm.

Additionally or alternatively, the organism may be a parasite. The parasite may ingest the conjugate. The parasite may be Plasmodium parasite, such as P. falciparum, P. vivax, P. ovate, P. malaria and P. knowlesi, or a Theileria parasite, such as T. annulata and T. parva, or a Phytophthora parasite, such as P. cinnamomi and P. agathidicida, or a Babesia parasite, such as B. bovis, or a Crithidia parasite, such as C. bombi, or a Lotmaria parasite, such as L. passim, or a Trypanosoma parasite, such as T. brucei, or a Toxoplasma parasite, such as T. gondii.

In another preferred embodiment, the organism is a parasite.

When the organism is a parasite, the parasite is preferably a Plasmodium parasite, such as P. vivax, P. ovate, P. malaria and P. knowlesi, or a Theileria parasite, such as T. annulata and T. parva, or a Phytophthora parasite, such as P. cinnamomi and P. agathidicida, or a Babesia parasite, such as B. bovis, or a Crithidia parasite, such as C. bombi, or a Lotmaria parasite, such as L. passim, or a Trypanosoma parasite, such as T. brucei, or a Toxoplasma parasite, such as T. gondii.

When the organism is a parasite, the parasite is more preferably a Theileria parasite, such as T. annulata and T. parva, or a Phytophthora parasite, such as P. cinnamomi and P. agathidicida, or a Babesia parasite, such as B. bovis, or a Crithidia parasite, such as C. bombi, or a Lotmaria parasite, such as L. passim, or a Toxoplasma parasite, such as T. gondii.

The organism may be a microbe, such as a bacterium. The microbe, such as bacterium, may ingest the conjugate. The microbe, such as bacterium, may be a Mycobacteria bacterium, such as M. tuberculosis, or an Escherichia bacterium, such as E. coli, or a Staphylococcus bacterium, such as S. aureus, or an Enterococcus bacterium, such as E. faecalis.

When the organism is a microbe, such as bacterium, the microbe is preferably a Mycobacteria bacterium, such as M. tuberculosis, or an Enterococcus bacterium, such as E. faecalis.

When the organism is a microbe, such as bacterium, the microbe is more preferably a Mycobacteria bacterium, such as M. tuberculosis.

The conjugate is able to pass through a cell wall of an organism.

The organism maybe unicellular or multicellular.

The methods of delivery may be performed in vivo or ex vivo. Thus, the organism may be located inside a human, insect, or animal, or the organism may be located outside a human, insect, or animal.

### Treatment

The term “treatment,” as used herein in the context of treating a condition, pertains generally to treatment and therapy, whether of a human, insect, or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients or subjects who have not yet developed the condition, but who are at risk of developing the condition, is encompassed by the term “treatment.”

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

The term “treatment” includes combination treatments and therapies, as described herein, in which two or more treatments or therapies are combined, for example, sequentially or simultaneously.

### Formulations

In one aspect, the present invention provides a pharmaceutical composition comprising a compound of formula (I) together with a pharmaceutically acceptable excipient, such as a carrier. The pharmaceutical composition may additionally comprise a second active agent. In an alternative embodiment, where a second agent is provided for use in therapy, the second agent may be separately formulated from the compound of formula (I). The comments below made in relation to the compound of formula (I) may therefore also apply to the second agent, as separately formulated.

While it is possible for the compound of formula (I) to be administered alone or together with the second agent, it is preferable to present it as a pharmaceutical formulation (e.g., composition, preparation, medicament) comprising at least one compound of formula (I), as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one compound of formula (I), as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound. The composition optionally further comprises the second active agent in a predetermined amount.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound of formula (I) with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, lozenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

### Dosage

Generally, the methods of the invention may comprise administering to a subject an effective amount of a compound of formula (I) to provide a biological effect.

It will be appreciated by one of skill in the art that appropriate dosages of the compound of formula (I), and compositions comprising the compound of formula (I), can vary from patient to patient. Determining the optimal dosage will generally involve the balancing of the level of therapeutic benefit against any risk or deleterious side effects. The selected dosage level will depend on a variety of factors including, but not limited to, the activity of the particular compound of formula (I), the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound of formula (I) and route of administration will ultimately be at the discretion of the physician, veterinarian, beekeeper, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of a compound of formula (I) is in the range of about 10 μg to about 250 mg (more typically about 100 μg to about 25 mg) per kilogram body weight of the subject per day. Where the compound of formula (I) is a salt, a prodrug, or the like, the amount administered is calculated on the basis of the parent compound and so the actual weight to be used is increased proportionately.

### Routes of Administration

A compound of formula (I), or a pharmaceutical composition comprising the compound of formula (I), may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orang-utan, gibbon), an insect (e.g. bee, bumblebee), or a human. Additionally or alternatively, the subject/patient may be a caprine (e.g. goat).

Furthermore, the subject/patient may be any of its forms of development, for example, a foetus. The present inventors have found that the conjugates of the invention may not be taken up by mammalian cells, and this includes rapidly dividing mammalian cells, such as those within the developing foetus.

Thus, the conjugates of the invention may be used to treat pregnant subjects and subjects who are intending to conceive.

In one preferred embodiment, the subject/patient is a human.

It is also envisaged that the invention may be practised on a non-human animal having a microbial infection. A non-human mammal may be a rodent. Rodents include rats, mice, guinea pigs, chinchillas and other similarly-sized small rodents used in laboratory research.

### Other Preferences

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “comprising” as used in this specification and claims means “consisting at least in part of”. When interpreting statements in this specification and claims which include the term “comprising”, other features besides the features prefaced by this term in each statement can also be present. Related terms such as “comprise” and “comprises” are to be interpreted in similar manner.

In this specification where reference has been made to external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

Unless context dictates otherwise, the descriptions and definitions of the features set out above are not limited to any particular aspect or embodiment of the invention and apply equally to all aspects and embodiments which are described. Where technically appropriate embodiments may be combined and thus the disclosure extends to all permutations and combinations of the embodiments provided herein.

Certain aspects and embodiments of the invention will now be illustrated by way of example and with reference to the figures described above.

### Examples

The following examples are provided solely to illustrate the present invention and are not intended to limit the scope of the invention, as described herein.

### General Synthesis Methods

Reactions involving air sensitive reagents and anhydrous solvents were performed in glassware that had been dried in an oven (130° C.). These reactions were carried out with the exclusion of air using an argon atmosphere. Acetonitrile, dichloromethane, diethyl ether, tetrahydrofuran and toluene were purified through a Pure Solv 400-5MD solvent purification system (Innovative Technology, Inc.). All reagents were used as received, unless otherwise stated. Solvents were evaporated under reduced pressure at 40° C. using a Buchi Rotavapor.

Microwave reactions were performed in a Biotage Initiator system.

Column chromatography was performed under pressure using silica gel (Fluoro Chem Silica LC 60A) as the stationary phase and HPLC grade solvents as eluent. Reactions were monitored by thin layer chromatography. TLC was performed on aluminium sheets pre-coated with silica gel (Merck or Fluorochem Silica Gel 60 F254). The plates were visualised by the quenching of UV fluorescence (λₘₐₓ 254 nm) and/or by staining with a KMnO₄ solution or acidic ethanolic anisaldehyde dip.

Proton magnetic resonance spectra (¹H NMR) and carbon magnetic resonance spectra (¹³C NMR) were recorded at 400 MHz and 100 MHz or at 500 MHz and 125 MHz using either a Bruker DPX Avance400 instrument or a Bruker AvanceIII500 instrument. Chemical shifts (δ) are reported in parts per million (ppm) and are referenced to the residual solvent peak. The order of citation in parentheses is (i) number of equivalent nuclei (by integration), (ii) multiplicity (s=singlet, d=doublet, t=triplet, q=quartet, m=multiplet, b=broad, dm=double multiplet, or a combination of these terms), and (iii) coupling constant (J) quoted in Hertz to the nearest 0.1 Hz.

IR spectra were obtained employing a Golden Gate™ attachment that uses a type IIa diamond as a single reflection element so that the IR spectrum of the compound (solid or liquid) could be detected directly (thin layer) without any sample preparation (Shimadzu FTIR-8400). Only significant absorptions are reported in wavenumbers.

UV-Vis absorption spectra were recorded using a Shimadzu UV-3600 UV-Vis-NIR spectrophotometer. A Brand® UV-Cuvette UV-Transparent spectrophotometry plastic cuvette with a 10 mm pathlength and 3 mL volume was used. Fluorescent emission spectra were recorded using a Shimadzu RF-5301 PC spectrofluorophotometer and Panorama fluorescence 1.1 software.

High resolution mass spectra were recorded by the analytical group of the Chemistry department at Glasgow University on a JEOL JMS-700 mass spectrometer by electrospray and chemical ionisation or on a Bruker microTOFq mass spectrometer by electrospray ionisation.

### Compound Syntheses—Intermediates

### (E)-2-(Trimethylsilyl)ethyl 3-(2,2,5,5- tetramethyl-1,3-dioxane-4-carboxamido)acrylate and (Z)-2-(Trimethylsilyl)ethyl 3-(2,2,5,5-tetramethyl-1,3-dioxane-4-carboxamido)acrylate

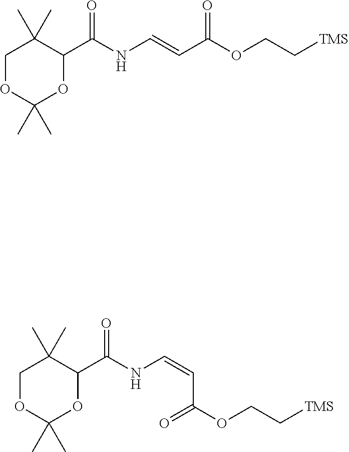 [SMILES: CC1(C)OCC(C)(C)C(C(=O)N/C=C/C(=O)OCC[Si](C)(C)C)O1 ; CC1(C)OCC(C)(C)C(C(=O)N/C=C\C(=O)OCC[Si](C)(C)C)O1]

2-(Trimethylsilyl)ethyl-2-(triphenylphosphoranylidene)acetate (0.84 g, 3.9 mmol) was dissolved in benzene (60 mL ) and N-formyl-2,2,5,5-tetramethyl-1,3-dioxane-4-carboxamide (R-form or S-form) (0.84 g, 3.9 mmol) was added. The solution was heated to 80° C. for 18 h then let cool to rt. The solvent was removed in vacuo to give the crude as a yellow oil. The crude was purified using column chromatography (10-20% EtOAc/petroleum ether) to give the enamide products as a separable mixture of cis (0.46 g, 33%) and trans (0.75 g, 54%) isomers as a yellow oil and white solid respectively. The NMR data obtained for this compound matches the literature data for this compound.

See also Sewell et al.

(E)-form: ¹H NMR (CDCl₃, 500 MHz) δ: 8.38 (1H, d, J=11.8 Hz), 7.97 (1H, dd, J=14.2, 11.8 Hz), 5.59 (1H, d, J=14.2 Hz), 4.24-4.18 (2H, m), 4.20 (1H, s), 3.72 (1H, d, J=11.8 Hz), 3.32 (1H, d, J=11.8 Hz), 1.52 (3H, s), 1.46 (3H, s), 1.06 (3H, s), 1.02 (2H, t, J=8.4 Hz), 1.01 (3H, s), 0.05 (9H, s); ¹³C NMR (CDCl₃, 125 MHz) δ: 169.4, 168.7, 137.2, 104.7, 100.0, 78.7, 72.2, 63.9, 34.9, 30.9, 23.3, 20.3, 20.1, 18.8, 0.0; IR νₘₐₓ (film)/cm⁻¹ 3295, 2957, 2897, 1688, 1638, 1476; HRMS (Cl) calcd for C₁₇H₃₂O₅NSi (M+H)+: m/z 358.2050, found m/z 358.2052; mp 126-127° C.

R-form: [α]_{D} +58.9 (c=1.1, CHCl₃, T=22.5° C.).

S-form: [α]_{D} −56.7 (c=0.6, CHCl₃, T=21.6° C.).

(Z)-form: ¹H NMR (CDCl₃, 500 MHz) δ: 11.08 (1H, d, J=11.6 Hz), 7.40 (1H, dd, J=11.6, 9.0 Hz), 5.15 (1H, d, J=8.9 Hz), 4.26-4.20 (2H, m), 4.21 (1H, s), 3.72 (1H, d, J=11.6 Hz), 3.32 (1H, d, J=11.6 Hz), 1.59 (3H, s), 1.47 (3H, s), 1.05 (3H, s), 1.04 (3H, s), 1.02 (2H, m), 0.05 (9H, s); ¹³C NMR (CDCl₃, 125 MHz) δ: 170.2, 169.8, 137.3, 100.8, 99.8, 79.1, 72.8, 63.7, 34.8, 30.8, 23.4, 20.5, 20.1, 18.9, 0.0; IR νₘₐₓ (film)/cm⁻¹ 3331,2959, 2858,1680, 1628, 1478; HRMS (Cl) calcd for C₁₇H₃₂O₅NSi (M+H)+: m/z 358.2050, found m/z 358.2054.

R-form: [α]_{D} +40.5 (c=1.0, CHCl₃, T=22.5° C.).

S-form: [α]_{D} −38.0 (c=0.8, CHCl₃, T=22.5° C.).

### (E)-3-(2,2,5,5-Tetramethyl-1,3-dioxane-4-carboxamido)acrylic Acid

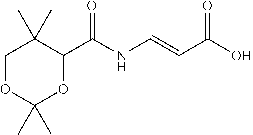 [SMILES: CC1(C)OCC(C)(C)C(C(=O)N/C=C/C(=O)O)O1]

This compound is also known from Sewell et al. (see compound 11).

(E)-2-(Trimethylsilyl)ethyl 3-(2,2,5,5-tetramethyl-1,3-dioxane-4-carboxamido)acrylate (R-form or S-form) (110 mg, 0.310 mmol) was dissolved in THF (20 mL ) and cooled to 0° C. before addition of TBAF (0.370 mL of a 1 M solution in THF, 0.370 mmol). The resultant pale yellow solution was allowed to stir for 16 h while warming up to room temperature before concentrating the reaction mixture in vacuo. Purification of the crude residue by flash column chromatography (0-2% MeOH/CH₂Cl₂) gave the acid product as a mixture with TBAF impurities. The crude product was dissolved in EtOAc (10 mL ) and water (10 mL ) before 1 M aqueous NaOH was added until the solution was pH 12. The resultant solution was stirred for 1 hr at room temperature before the aqueous layer was collected and diluted with EtOAc (15 mL ) before being 1 M HCl was added until pH 5. The mixture was stirred for a further 1 h at room temperature before the layers were separated and the aqueous phase was extracted with EtOAc (2×10 mL ). The combined organics were washed with brine (20 mL ), dried (Na₂SO₄) and filtered before solvent was removed in vacuo to give the acid product (64 mg, 79%) as a white solid.

¹H NMR (CD₃OD, 400 MHz) δ: 7.95 (1H, d, J=14.3 Hz), 5.86 (1H, J=14.3 Hz), 4.31 (1H, s), 3.80 (1H, d, J=11.6 Hz), 3.32 (1H, d, J=11.6 Hz), 1.53 (3H, s), 1.45 (3H, s), 1.04 (3H, s), 1.03 (3H, s); ¹³C NMR (CD₃OD, 100 MHz) δ: 171.5, 170.9, 138.5, 104.0, 100.8, 78.6, 72.2, 34.3, 29.5, 22.1, 19.3, 19.0; IR νₘₐₓ (film)/cm⁻¹ 3318, 3094, 2963, 2878, 1670, 1636; HRMS (Cl) calcd for C₁₂H₂₀O₅N (M+H)+: m/z 258.1341, found m/z 258.1346; mp 185-186° C.

R-form: [α]_{D} +81.3 (c=0.5, CHCl₃, T=22.7° C.).

S-form: [α]_{D} −77.6 (c=1.2, CHCl₃, T=22.8° C.).

### (Z)-3-(2,2,5,5-Tetramethyl-1,3-dioxane-4-carboxamido)acrylic Acid

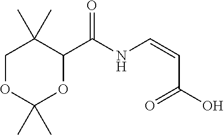 [SMILES: CC1(C)OCC(C)(C)C(C(=O)N/C=C\C(=O)O)O1]

(Z)-2-(Trimethylsilyl)ethyl 3-(2,2,5,5-tetramethyl-1,3-dioxane-4-carboxamido)acrylate (R-form or S-form) (310 mg, 0.868 mmol) was dissolved in a mixture of THF (6 mL ) and water (0.150 mL ) before being cooled to 0° C. TBAF (1.30 mL of a 1 M solution in THF, 1.30 mmol) was added via syringe at 0° C. and then left to stir at room temperature for 16 h. Solvent was then removed in vacuo to give the crude product as a pale yellow oil. Purification of the crude residue by flash column chromatography (0-2% MeOH/CH₂Cl₂) gave the desired acid product (178 mg, 80%) as a colourless oil.

¹H NMR (CDCl₃, 500 MHz) δ: 11.25, (1H, d, J=11.8 Hz), 7.54 (1H, dd, J=11.8, 8.8 Hz), 5.22 (1H, d, J=8.9 Hz), 4.21 (1H, s), 3.73 (1H, d, J=11.7 Hz), 3.33 (1H, d, J=11.7 Hz), 1.53 (3H, s), 1.46 (3H, s), 1.05 (3H, s), 1.03 (3H, s); ¹³C NMR (CDCl₃, 125 MHz) δ: 173.6, 168.8, 138.6, 99.3, 96.6, 77.2, 71.2, 33.2, 29.2, 21.8, 19.0, 18.6; IR νₘₐₓ (film)/cm⁻¹ 3302, 2993, 2940, 2870, 1678, 1601; HRMS (Cl) calcd for C₁₂H₂₀O₅N (M+H)+: m/z 258.1341, found m/z 258.1339.

R-form: [α]_{D} +51.0 (c=0.5, CHCl₃, T=22.7° C.).

S-form: [α]_{D} −47.1 (c=0.8, CHCl₃, T=21.7° C.).

### (Z)-3-(2,4-Dihydroxy-3,3-dimethylbutanamido)acrylic Acid

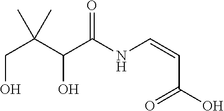

(Z)-3-(2,2,5,5-Tetramethyl-1,3-dioxane-4-carboxamido)acrylic acid (R-form or S-form) (40 mg, 0.160 mmol) was treated with BiCl₃ (5 mg, 16 μmol) in MeCN (1 mL ) and H₂O (50 μL). Purification of the crude residue by flash column chromatography (0-5% MeOH/CH₂Cl₂) gave the diol product (9 mg, 26%) as a colourless oil.

¹H NMR (CDCl₃, 500 MHz) δ: 7.43 (1H, d, J=8.9 Hz), 5.17 (1H, d, J=8.9 Hz), 4.05 (1H, s), 3.49 (1H, d, J=10.9 Hz), 3.41 (1H, d, J=10.9 Hz), 0.94 (3H, s), 0.93 (3H, s); ¹³C NMR (CDCl₃, 125 MHz) δ: 174.6, 171.4, 137.3, 98.8, 76.8, 69.8, 40.8, 21.3, 20.5; IR νₘₐₓ (film)/cm⁻¹ 3306, 2967, 2940, 2832, 1670; HRMS (Cl) calcd for C₉H₁₅O₅N (M+H)+: m/z 218.1028, found m/z 218.1029.

The R-form is known.

S-form: [α]_{D} −30.8 (c=1.4, MeOH, T=25.5° C.).

### (E)-N-(3-(3-Hydroxypropylamino)-3-oxoprop-1-enyl)-2,2,5,5-tetramethyl-1,3-dioxane-4-carboxamide

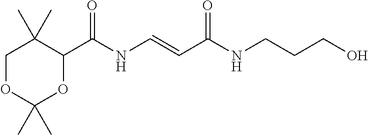 [SMILES: CC1(C)OCC(C)(C)C(C(=O)N/C=C/C(=O)NCCCO)O1]

(E)-3-(2,2,5,5-Tetramethyl-1,3-dioxane-4-carboxamido)acrylic acid (50 mg, 0.195 mmol) was dissolved in CH₂Cl₂ (0.5 mL ) and the resulting solution was treated with HBTU (110 mg, 0.290 mmol), 3-amino-propan-1-ol (22 μL, 0.290 mmol) and DIPEA (51 μL, 0.290 mmol). The resultant solution was heated under microwave conditions at 80° C. for 2.5 h before removing solvent in vacuo to afford the crude product. Purification of the crude residue by flash column chromatography (0-5% MeOH/EtOAc) gave the pantothenamide product (20 mg, 33%) as a white solid.

¹H NMR (CDCl₃, 500 MHz) δ: 8.35 (1H, d, J=10.8 Hz), 7.80 (1H, dd, J=13.8, 10.8, Hz), 5.98 (1H, t, J=6.0 Hz), 5.86 (1H, d, J=13.8 Hz), 4.20 (1H, s), 3.73 (1H, d, J=11.7 Hz), 3.66-3.63 (2H, m), 3.53-3.48 (3H, m), 3.33 (1H, d, J=11.7 Hz), 1.74-1.69 (2H, m), 1.52 (3H, s), 1.47 (3H, s), 1.06 (3H, s), 1.01 (3H, s); ¹³C NMR (CDCl₃, 125 MHz) δ: 168.2, 167.5, 133.3, 105.7, 99.5, 77.3, 71.3, 59.2, 36.3, 33.4, 32.5, 29.5, 21.9, 18.8, 18.7; IR νmax (film)/cm⁻¹ 3275, 2998, 2953, 2876, 1705, 1659; HRMS (Cl) calcd for C₁₅H₂₇O₅N₂ (M+H)+: m/z 315.1920, found m/z 315.1917; m.p. 165-166° C.

### (Z)—N-(3-(3-Hydroxypropylamino)-3-oxoprop-1-enyl)-2,2,5,5-tetramethyl-1,3-dioxane-4-carboxamide

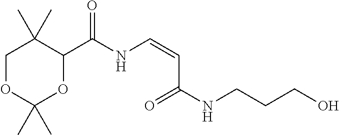 [SMILES: CC1(C)OCC(C)(C)C(C(=O)N/C=C\C(=O)NCCCO)O1]

(I)-3-(2,2,5,5-Tetramethyl-1,3-dioxane-4-carboxamido)acrylic acid (50 mg, 0.195 mmol) was treated following the procedure for the synthesis of the corresponding (E)-form described above with BTFFH (92 mg, 0.290 mmol), 3-amino-propan-1-ol (22 μL, 0.290 mmol) and DIPEA (51 μL, 0.290 mmol). Purification of the crude residue by flash column chromatography (0-5% MeOH/EtOAc) gave the pantothenamide product (31 mg, 57%) as a white solid.

¹H NMR ((CD₃)₂CO, 500 MHz) δ: 11.91 (1H, d, J=11.1 Hz), 7.38 (1H, brs), 7.24 (1H, dd, J=11.1, 8.9 Hz), 5.25 (1H, d, J=8.9 Hz), 4.28 (1H, s), 3.87 (1H, brs), 3.81 (1H, d, J=11.6 Hz), 3.58 (2H, t, J=6.0 Hz), 3.41-3.35 (2H, m), 3.31 (1H, d, J=11.6 Hz), 1.70-1.65 (2H, m), 1.60 (3H, s), 1.50 (3H, s), 1.04 (3H, s), 1.00 (3H, s); ¹³C NMR ((CD₃)₂CO), 125 MHz) δ: 169.3, 169.0, 133.3, 101.4, 99.7, 77.7, 71.6, 59.5, 36.3, 33.7, 33.5, 29.5, 22.0, 19.3, 19.0; IR μmax (film)/cm⁻¹ 3243, 2974, 2930, 2842, 1672, 1612; HRMS (Cl) calcd for C₁₅H₂₆O₅N₂ (M+H)+: m/z 315.1918, found m/z 315.1920; m.p. 80-81° C.

### (E)-N-(3-(5-Hydroxypentylamino)-3-oxoprop-1-enyl)-2,2,5,5-tetramethyl-1,3-dioxane-4-carboxamide

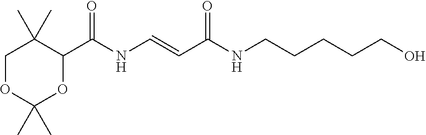 [SMILES: CC1(C)OCC(C)(C)C(C(=O)N/C=C/C(=O)NCCCCCO)O1]

(E)-3-(2,2,5,5-Tetramethyl-1,3-dioxane-4-carboxamido)acrylic acid (50 mg, 0.195 mmol) was treated following the procedure for the synthesis of (E)-N-(3-(3-hydroxypropylamino)-3-oxoprop-1-enyl)-2,2,5,5-tetramethyl-1,3-dioxane-4-carboxamide with HBTU (110 mg, 0.290 mmol), 5-amino-pentan-1-ol (100 mg, 0.971 mmol) and DIPEA (51 μL, 0.290 mmol). Purification of the crude residue by flash column chromatography (0-5% MeOH/EtOAc) gave the pantothenamide product (12 mg, 18%) as a colourless oil.

¹H NMR (CDCl₃, 400 MHz) δ: 8.31 (1H, d, J=11.0 Hz), 7.78 (1H, dd, J=13.9, 11.0 Hz), 5.85 (1H, d, J=13.9 Hz), 5.61 (1H, t, J=5.4 Hz), 4.21 (1H, s), 3.74 (1H, d, J=11.8 Hz), 3.67 (2H, t, J=6.0 Hz), 3.39-3.34 (2H, m), 3.34 (1H, d, J=11.8 Hz), 1.78 (1H, brs), 1.65-1.55 (4H, m), 1.53 (3H, s), 1.48 (3H, s), 1.48-1.41 (2H, m), 1.07 (3H, s), 1.02 (3H, s); ¹³C NMR (CDCl₃, 125 MHz) δ: 168.2, 166.3, 132.8, 106.3, 99.5, 77.2, 71.3, 62.6, 39.4, 33.4, 32.2, 29.5, 29.4, 23.0, 21.9, 18.8, 18.7; IR νmax (film)/cm⁻¹ 3264, 2991,2934, 2868, 1701, 1661; HRMS (Cl) calcd for C₁₇H₃₁O₅N₂ (M+H)+: m/z 343.2238, found m/z 343.2233.

### (Z)—N-(3-(5-Hydroxypentylamino)-3-oxoprop-1-enyl)-2,2,5,5-tetramethyl-1,3-dioxane-4-carboxamide

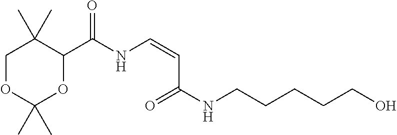 [SMILES: CC1(C)OCC(C)(C)C(C(=O)N/C=C\C(=O)NCCCCCO)O1]

(Z)-3-(2,2,5,5-Tetramethyl-1,3-dioxane-4-carboxamido)acrylic acid (50 mg, 0.195 mmol) was treated following the procedure for the synthesis of (E)-N-(3-(3-hydroxypropylamino)-3-oxoprop-1-enyl)-2,2,5,5-tetramethyl-1,3-dioxane-4-carboxamide with BTFFH (92 mg, 0.290 mmol), 5-amino-pentan-1-ol (30 mg, 0.290 mmol) and DIPEA (51 μL, 0.290 mmol). Purification of the crude residue by flash column chromatography (0-5% MeOH/EtOAc) gave the pantothenamide product (27 mg, 40%) as a colourless oil.

¹H NMR ((CD₃)₂CO, 400 MHz) δ: 11.92 (1H, d, J=11.0 Hz), 7.27 (1H, brs), 7.22 (1H, dd, J=11.0, 8.9 Hz), 5.24 (1H, d, J=8.9 Hz), 4.27 (1H, s), 3.80 (1H, d, J=11.8 Hz), 3.58-3.54 (2H, m), 3.29-3.24 (2H, m), 3.31 (1H, d, J=11.8 Hz), 1.78 (1H, brs), 1.58-1.50 (4H, m), 1.52 (3H, s), 1.50 (3H, s), 1.46-1.40 (2H, m), 1.04 (3H, s), 1.00 (3H, s); ¹³C NMR ((CD₃)₂CO), 100 MHz) δ: 169.0, 168.6, 133.0, 101.8, 99.8, 77.8, 71.6, 62.3, 39.4, 33.7, 33.4, 30.2, 29.9, 24.1, 22.1, 19.4, 19.0; IR νₘₐₓ (film)/cm⁻¹ 3293, 2992, 2937, 2868, 1652, 1609; HRMS (Cl) calcd for C₁₇H₃₁O₅N₂ (M+H)+: m/z 343.2230, found m/z 343.2233.

### (R,E)-N-(3-(But-3-ynylamino)-3-oxoprop-1-enyl)-2,2,5,5-tetramethyl-1,3-dioxane-4-carboxamide

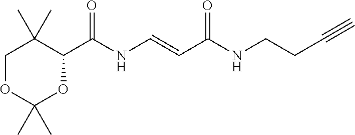 [SMILES: C#CCCNC(=O)/C=C/NC(=O)[C@@H]1OC(C)(C)OCC1(C)C]

(R,E)-3-(2,2,5,5-Tetramethyl-1,3-dioxane-4-carboxamido)acrylic acid (100 mg, 0.389 mmol) was treated following the procedure for the synthesis of (E)-N-(3-(3-hydroxypropylamino)-3-oxoprop-1-enyl)-2,2,5,5-tetramethyl-1,3-dioxane-4-carboxamide with BTFFH (184 mg, 0.584 mmol), 1-amino-3-butyne (46 μL, 0.584 mmol) and DIPEA (102 μL, 0.584 mmol). Purification of the crude residue by flash column chromatography (50-60% EtOAc/petroleum ether) gave the pantothenamide product (118 mg, 99%) as a white solid.

¹H NMR ((CD₃)₂CO, 500 MHz) δ: 9.35 (1H, d, J=11.1 Hz), 7.83 (1H, dd, J=13.9, 11.1 Hz), 7.18 (1H, brs), 5.95 (1H, d, J=13.9 Hz), 4.28 (1H, s), 3.78 (1H, d, J=11.4 Hz), 3.40-3.36 (2H, m), 3.29 (1H, d, J=10.6 Hz), 2.39 (2H, td, J=6.9, 2.6 Hz), 2.36 (1H, t, J=2.6 Hz), 1.47 (3H, s), 1.40 (3H, s), 1.03 (3H, s), 1.00 (3H, s). 13C NMR ((CD₃)₂CO), 125 MHz) δ: 169.3, 166.9, 133.8, 106.7, 100.0, 82.7, 77.9, 71.7, 70.7, 39.1, 33.9, 29.9, 22.1, 19.9, 19.2, 19.0; IR νₘₐₓ (film)/cm⁻¹ 3309, 3287, 2996, 2944, 2889, 2871, 1657, 1616; HMRS (EI) calcd for C₁₆H₂₄O₄N₂ M+: m/z 308.1736, found m/z 308.1739; m.p. 153-154° C.; [α]D +73.9 (c=0.5, (CH₃)₂CO, T=30.0° C.).

The (S,E)-form may be prepared in a similar manner from (S,E)-3-(2,2,5,5-Tetramethyl-1,3-dioxane-4-carboxamido)acrylic acid. [α]_{D} −65.5 (c=1.1, (CH₃)₂CO, T=24.4° C.).

### (R,E)-N-(3-(But-3-ynylamino)-3-oxoprop-1-enyl)-2,4-dihydroxy-3,3-dimethylbutan-amide

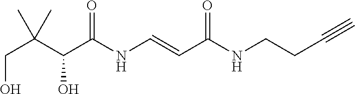 [SMILES: C#CCCNC(=O)/C=C/NC(=O)[C@H](O)C(C)(C)CO]

(R,E)-N-(3-(But-3-ynylamino)-3-oxoprop-1-enyl)-2,2,5,5-tetramethyl-1,3-dioxane-4-carboxamide (110 mg, 0.357 mmol) was dissolved in a mixture of MeCN (1 mL ) and H₂O (50 μL). BiCl₃ (11 mg, 0.035 mmol) was then added, resulting in a milky suspension that was stirred for 18 h at rt. The reaction mixture was quenched with a few drops of saturated aqueous NaHCO₃ followed by filtration through a pad of Celite® that was subsequently washed with EtOAc (10 mL ). The eluant was concentrated in vacuo to give the crude product. Purification of the crude residue by flash column chromatography (5-7% MeOH/CH₂Cl₂) gave the diol product (49 mg, 51%) as a white solid.

¹H NMR ((CD₃)₂CO, 400 MHz) δ: 9.55 (1H, d, J=11.3 Hz), 7.87 (1H, dd, J=14.0, 11.3 Hz), 7.27 (1H, brs), 6.00 (1H, d, J=14.0 Hz), 4.09 (1H, d, J=5.0 Hz), 4.03 (1H, t, J=5.5 Hz), 3.56 (1H, br s), 3.49 (1H, dd, J=10.5, 5.5 Hz), 3.43 (1H, dd, J=10.6, 5.5 Hz), 3.41-3.35 (2H, m), 2.38 (2H, td, J=6.9, 2.6 Hz), 2.37-2.35 (1H, m), 0.94 (3H, s), 0.93 (3H, s); ¹³C NMR ((CD₃)₂CO), 100 MHz) δ: 172.9, 167.1, 134.2, 106.2, 82.7, 77.5, 70.7, 70.4, 40.3, 39.2, 21.3, 20.7, 19.9; IR νₘₐₓ (film)/cm⁻¹ 3368, 3309, 3247, 2965, 2920, 2861, 1691, 1652; HMRS (EI) calcd for C₁₃H₁₉O₄N₂ (M−H)+: m/z 267.1350, found m/z 267.1344; m.p. 171-172° C.; [α]_{D} +78.1 (c=1.1, (CH₃)₂CO, T=30.0° C.).

### (Z)—N-(3-(But-3-yn-1-ylamino)-3-oxoprop-1-en-1-yl)-2,2,5,5-tetramethyl-1,3-dioxane-4-carboxamide

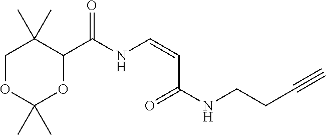 [SMILES: C#CCCNC(=O)/C=C\NC(=O)C1OC(C)(C)OCC1(C)C]

(Z)-3-(2,2,5,5-Tetramethyl-1,3-dioxane-4-carboxamido)acrylic acid (R-form or S-form) (125 mg, 0.49 mmol) was dissolved in CH₂Cl₂ (1 mL ) in a 0.5-2 mL microwave vial with a magnetic stirring bar. BTFFH (233 mg, 0.74 mmol) was added then DIPEA (150 μL, 0.74 mmol) and 1-amino-3-butyne (61 μL, 0.74 mmol) via micropipette. The vial was capped and heated to 80° C. under microwave conditions for 2.5 h. The solvent was then removed in vacuo to give the crude as a pale yellow oil. This was purified by flash column chromatography (10-25% EtOAc/petroleum ether) to give the alkyne product as a white solid (110 mg, 73%).

¹H NMR (CDCl₃, 400 MHz) δ: 11.66 (1H, d, J=10.9 Hz), 7.31 (1H, dd, J=10.9, 8.9 Hz), 5.74 (1H, brs), 5.03 (1H, d, J=8.9 Hz), 4.20 (1H, s), 3.72 (1H, d, J=11.6 Hz), 3.48 (2H, m), 3.32 (1H, d, J=11.6 Hz), 2.44 (2H, dt, J=6.5, 2.6 Hz), 2.03 (1H, t, J=2.6 Hz), 1.61 (3H, s), 1.47 (3H, s), 1.05 (6H, s); ¹³C NMR (CDCl₃, 100 MHz) δ: 168.9, 167.7, 133.7, 100.2, 99.3, 81.5, 77.3, 71.4, 70.2, 37.6, 33.3, 29.4, 21.9, 19.5, 19.1, 18.7; IR νₘₐₓ (film)/cm⁻¹ (neat): 3294, 2955, 1656, 1467, 1249; HRMS (ESI) calcd for C₁₆H₂₃N₂O₄ (M−H) c m/z 307.1663 found m/z 307.1658; m.p. 122-125° C.

R-form: [α]_{D} +20.6 (C=1.0, (CH₃)₂CO, T=24.4° C.).

S-form: [α]_{D} −23.1 (c=1.4, (CH₃)₂CO, T=24.4° C.).

### (Z)-2-Methyl-3-[((R)-2,2,5,5-tetramethyl-[1,3]dioxane-4-carbonyl)-amino]-acrylic acid ethyl ester, and (E)-2-Methyl-3-[((R)-2,2,5,5-tetramethyl-[1,3]dioxane-4-carbonyl)-amino]-acrylic Acid Ethyl Ester

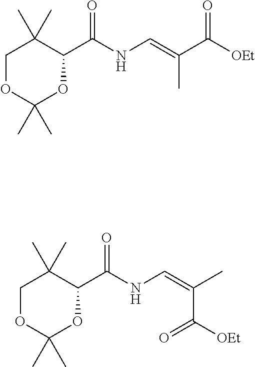 [SMILES: CCOC(=O)/C(C)=C/NC(=O)[C@@H]1OC(C)(C)OCC1(C)C ; CCOC(=O)/C(C)=C\NC(=O)[C@@H]1OC(C)(C)OCC1(C)C]

Formyl compound 9 as described by Sewell et a/. (186 mg) was added to a solution of 1-carbethoxyethylidene triphenylphosphorane in benzene to generate the enamide product (175 mg, 65%) as a mixture of E and Z isomers, E:Z ratio 3:1. In a general procedure a solution of the formyl compound (1.0 mmol) in benzene (10 mL ) was treated with ethoxycarbonylmethylene triphenylphosphorane (3.0 mmol) and the resulting mixture heated to 95° C. for 19 hours. Upon reaction completion as indicated by TLC analysis, the solvent was removed under vacuum. The crude residue was then purified by flash column chromatography (silica gel, 10% to 30% EtOAc in 40-60 petroleum ether) to afford the desired enamides.

Z-form: ¹H NMR (400 MHz, CDCl₃) δ: 10.90 (1H, bd, J=12.4 Hz), 7.26 (1H, dd, J=11.5, 1.3 Hz), 4.18 (2H, qd, J=7.2, 1.4 Hz), 4.13 (1H, s), 3.66 (1H, d, J=11.7 Hz), 3.26 (1H, d, J=11.7 Hz), 1.79 (3H, d, J=1.3 Hz), 1.51 (3H, s), 1.39 (3H, s), 1.25 (3H, t, J=7.1 Hz), 0.98 (3H, s), 0.96 (3H, s). ¹³C NMR (100 MHz, CDCl₃) δ: 167.6, 167.3, 131.3, 105.5, 98.2, 75.7, 70.4, 60.1, 32.2, 28.4, 20.9, 18.0, 17.6, 15.4, 13.4. IR νₘₐₓ (film)/cm⁻¹ 3410, 3036, 2992, 1695, 1652. HRMS calcd for C₁₅H₂₅O₅N (M+): 299.1733. Found 299.1735. [α]_{D} +62.1 (c=1.1, CHCl₃).

E-form: ¹H NMR (400 MHz, CDCl₃) δ: 8.25 (1H, bd, J=12.3 Hz), 7.86 (1H, dq, J=12.3, 1.4 Hz), 4.14 (1H, s), 4.10 (2H, qd, J=7.2, 1.2 Hz), 3.64 (1H, d, J=11.8 Hz), 3.22 (1H, d, J=11.8 Hz), 1.71 (3H, d, J=1.4 Hz), 1.41 (3H, s), 1.39 (3H, s), 1.25 (3H, t, J=7.1 Hz), 0.97 (3H, s), 0.91 (3H, s). ¹³C NMR (100 MHz, CDCl₃) δ: 168.0, 167.4, 130.1, 109.2, 99.3, 77.2, 71.2, 60.5, 33.2, 29.4, 21.8, 18.8, 18.6, 14.4, 10.4. IR νₘₐₓ (film)/cm⁻¹ 3410, 3028, 2992, 1695, 1652. HRMS calcd for C₁₅H₂₅O₅N (M+): 299.1733. Found 299.1735. [α]_{D} +40.7 (c=1.1, CHCl₃).

### (Z)—N-(2-Bromovinyl)-2,2,5,5-tetramethyl-1,3-dioxane-4-carboxamide

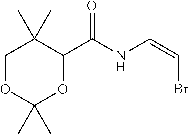 [SMILES: CC1(C)OCC(C)(C)C(C(=O)N/C=C\Br)O1]

(Bromomethyl)triphenylphosphonium bromide (8.72 g, 20.0 mmol) and potassium tert-butoxide (2.24 g, 20.0 mmol) were dried under vacuum for 10 min before cooling the flask to 0° C. and adding THF (40 mL ) to give a bright yellow suspension. The mixture was stirred for 1.5 h before adding a solution of N-formyl-2,2,5,5-tetramethyl-1,3-dioxane-4-carboxamide (1.09 g, 5.0 mmol) in THF (5.0 mL ) and leaving to stir coming to room temperature for 16 h. The reaction mixture was diluted with hexane (30 mL ) and H₂O (30 mL ) before filtering to remove solids. The aqueous was extracted with EtOAc (3×30 mL ), before washing combined organics with brine (45 mL ), drying (Na₂SO₄), filtering and removing solvent in vacuo to afford the crude as a brown oil. Purification of the crude residue by flash column chromatography (5-10% EtOAc/petroleum ether) afforded a mixture of the dibromo and cis-bromo enamides. The mixture was then dissolved in EtOAc (20 mL ). Pd(PPh₃)₄ (288 mg, 0.249 mmol) was added, followed by Bu₃SnH (0.670 mL , 2.49 mmol) before allowing to stir at room temperature for 16 h. The mixture was diluted with hexane (10 mL ) before filtering through Celite® and removing solvent in vacuo to afford the crude as a brown oil. Column chromatography (2-4% EtOAc/petroleum ether) afforded the cis-bromo enamide (607 mg, 41% over two steps) as a pale yellow oil.

¹H NMR (CDCl₃, 400 MHz) δ: 8.61 (1H, d, J=11.2 Hz), 7.37 (1H, dd, J=11.2, 5.9 Hz), 5.58 (1H, d, J=5.9 Hz), 4.20 (1H, s), 3.75 (1H, d, J=11.8 Hz), 3.35 (1H, d, J=11.8 Hz), 1.55 (3H, s), 1.49 (3H, s), 1.08 (3H, s), 1.06 (3H, s); ¹³C NMR (CDCl₃, 100 MHz) δ: 167.3, 124.2, 99.3, 89.8, 77.2, 71.3, 33.2, 29.4, 21.9, 18.9, 18.7; IR νₘₐₓ (film)/cm⁻¹ 3390, 2993, 2959, 2872, 1699, 1643.

### (Z)—N-(2-Bromovinyl)-2,2,5,5-pentamethyl-1,3-dioxane-4-carboxamide

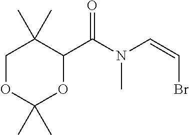 [SMILES: CN(/C=C\Br)C(=O)C1OC(C)(C)OCC1(C)C]

(Bromomethyl)triphenylphosphonium bromide (3.27 g, 7.50 mmol) and potassium tert-butoxide (300 mg, 7.50 mmol) were dried under vacuum for 10 min before cooling the flask to 0° C. and adding THF (10 mL ) to give a bright yellow suspension. The mixture was stirred for 1.5 h before adding a solution of N-formyl-N,2,2,5,5-pentamethyl-1,3-dioxane-4-carboxamide (345 mg, 1.50 mmol) in THF (2.0 mL ) and leaving to stir coming to room temperature for 16 h. The reaction mixture was diluted with hexane (10 mL ) and H₂O (10 mL ) before filtering to remove solids. The aqueous was extracted with EtOAc (3×15 mL), before washing combined organics with brine (20 mL), drying (Na₂SO₄), filtering and removing solvent in vacuo to afford the crude product as a brown oil. Column chromatography (5-10% EtOAc/petroleum ether) afforded a mixture of the dibromo compound and an unidentified impurity. The mixture was then dissolved in EtOAc (5 mL). Pd(PPh₃)₄ (59 mg, 0.051 mmol) was added, followed by Bu₃SnH (0.164 mL, 0.612 mmol) before allowing to stir at room temperature for 16 h. The mixture was diluted with hexane (5 mL) before filtering through Celite® and removing solvent in vacuo to afford the crude product as a brown oil. Column chromatography (5-20% EtOAc/petroleum ether) afforded the cis-bromo enamide (97 mg, 21% over two steps) as a pale yellow oil.

¹H NMR (CDCl₃, 400 MHz) δ: 7.14 (1H, d, J=5.5 Hz), 6.03 (1H, d, J=5.9 Hz), 4.32 (1H, s), 3.61 (1H, d, J=11.4 Hz), 3.31 (1H, d, J=11.4 Hz), 3.13 (3H, s), 1.46 (3H, s), 1.42 (3H, s), 1.26 (3H, s), 0.90 (3H, s); ¹³C NMR (CDCl₃, 100 MHz) δ: 167.1, 134.6, 99.5, 93.5, 77.2, 72.7, 33.7, 33.5, 29.4, 21.8, 19.4, 18.4; IR νₘₐₓ (film)/cm⁻¹ 3092, 2959, 2935, 2859, 1663.

### (Z)-2,2,5,5-Pentamethyl-N-(4-phenylbut-1-en-3-ynyl)-1,3-dioxane-4-carboxamide

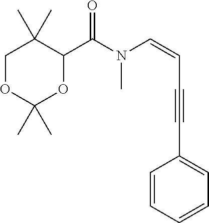 [SMILES: CN(/C=C\C#Cc1ccccc1)C(=O)C1OC(C)(C)OCC1(C)C]

(Z)—N-(2-Bromovinyl)-N,2,2,5,5-pentamethyl-1,3-dioxane-4-carboxamide (48 mg, 0.157 mmol) was dissolved in MeCN (1 mL ) before addition of phenylacetylene (35 μL, 0.314 mmol), Et₃N (87 μL, 0.628 mmol), CuI (6 mg, 31 μmol) and finally Pd(PPh₃)₄ (18 mg, 16 μmol). The mixture was stirred for 16 h at room temperature before filtering through Celite® with 20% EtOAc/hexane to afford the crude as a brown solid. Purification of the crude residue by flash column chromatography (2-5% EtOAc/petroleum ether) afforded the eneyne product (29 mg, 59%) as a pale yellow oil.

¹H NMR (CDCl₃, 400 MHz, 55° C.) δ: 7.42-7.40 (2H, m), 7.33-7.30 (3H, m), 7.05 (1H, d, J=8.7 Hz), 5.15 (1H, d, J=8.7 Hz), 4.48 (1H, s), 3.65 (1H, d, J=11.5 Hz), 3.55 (3H, brs), 3.35 (1H, d, J=11.5 Hz), 1.48 (3H, s), 1.30 (3H, s), 0.94 (3H, s); ¹³C NMR (CDCl₃, 100 MHz, 55° C.) δ: 167.8, 136.9, 131.1, 128.3, 128.2, 123.5, 99.6, 94.7, 93.2, 85.8, 76.6, 72.7, 34.2, 33.7, 29.1, 21.8, 19.2, 18.5; IR νₘₐₓ (film)/cm⁻¹ 2955, 2929, 2958, 2859, 1684, 1619.

### Compound Syntheses—Radiolabelled Intermediate

### 1,2-¹³C₃-2-(Trimethylsilyl)ethyl 2-bromoacetate

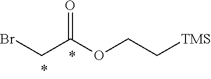 [SMILES: C[Si](C)(C)CCOC(=O)CBr]

¹³C₂-Bromoacetic acid (284) (890 mg, 6.41 mmol) was dissolved in CH₂Cl₂ and cooled to 0° C. before sequential addition of 2-trimethylsilyl ethanol (1.83 mL , 12.8 mmol), a catalytic amount of DMAP and finally portion-wise addition of DCC (1.39 g, 6.73 mmol). The resultant solution was allowed to stir for 2 h before filtering through a bed of Celite®. The Celite® pad was washed with 20% EtOAc/hexane (200 mL ), and the eluent then washed with aqueous saturated NaHCO₃ (150 mL ), water (150 mL ) and brine (150 mL ). The solution was then dried (Na₂SO₄) and solvent removal in vacuo gave the crude product as a yellow oil. Purification of the crude residue by flash column chromatography (2-4% EtOAc/hexane) afforded the ester product (1.05 g, 68%) as a pale yellow oil.

¹H NMR (CDCl₃, 400 MHz) δ: 4.29-4.24 (2H, m), 4.00-3.61 (2H, dd, J=152.8, 4.4 Hz), 1.07-1.00 (2H, m), 0.06 (9H, s); ¹³C NMR (CDCl₃, 100 MHz) δ: 172.6 (d, J=49.0 Hz), 66.3, 27.6 (d, J=49.0 Hz), 18.7, 0.0; IR νmax (film)/cm-1 2955, 2899, 1693, 1249; HRMS (ESI) calcd for C₅¹³C₂H₁₅O₂BrNaSi (M+Na)+: m/z 262.9984, found m/z 262.9977.

### 1,2-¹³C₂-2-(Trimethylsilylethoxycarbonylmethyl) triphenylphosphonium bromide

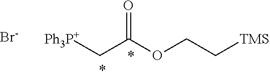

1,2-¹³C₂-2-(Trimethylsilyl)ethyl 2-bromoacetate (1.05 g, 4.38 mmol) was dissolved in toluene (20 mL ). Triphenylphosphine (1.15 g, 4.38 mmol) was added, and the resultant solution allowed to stir at room temperature for 16 h. At this time, the reaction mixture was thick with a white precipitate. A further 10 mL portion of toluene was added before sonicating and stirring for a further 1 h at rt. The white precipitate was collected by filtration and washed with toluene (2×20 mL before dissolving in CH₂Cl₂ (50 mL ). The solution was washed with brine (50 mL ), dried (Na₂SO₄) and concentrated in vacuo to afford the phosphonium salt (1.42 g, 65%) as a white solid.

¹H NMR (CDCl₃, 400 MHz) δ: 7.98-7.93 (6H, m), 7.85-7.80 (3H, m), 7.74-7.69 (6H, m), 5.83-5.44 (2H, ddd, J=134.0, 17.0, 9.0 Hz), 4.12-4.07 (2H, m), 0.90-0.87 (2H, m), 0.00 (9H, s); ¹³C NMR (CDCl₃, 100 MHz) δ: 166.5 (d, J=57.5 Hz), 136.7, 135.7, 131.9, 67.1, 35.0 (d, J=57.5 Hz), 18.8, 0.00; IR νₘₐₓ (film)/cm⁻¹ 3478, 3405, 2954, 2802, 1674; m.p. 90-91° C.

### ¹³C-1H-Benzotriazole-1-carboxaldehyde

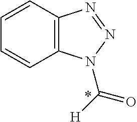 [SMILES: O=Cn1nnc2ccccc21]

Acetic anhydride (1.34 mL , 14.2 mmol) and ¹³C-formic acid (0.800 mL , 21.3 mol) were heated at 50° C. for 3 h. At this time, analysis of the crude NMR spectra allowed calculation of the mass ratio of ¹³C1-acetic formic anhydride. In this case, 9.94 mmoles of the mixed anhydride had been formed. Benzotriazole (1.06 g, 8.92 mmol) was dissolved in THF (5 mL ) at −10° C. and the resulting solution was treated with the crude mixed anhydride mixture via syringe addition and left to stir for 1 h. Solvent was removed under vacuum, and the crude white solid formed azeotroped with CHCl₃ to remove excess formic acid to give the formylated product (1.06 g, 99%) as a white solid.

¹H NMR (500 MHz, CDCl₃) δ: 10.08-9.54 (1H, d, J=220.0 Hz), 8.28 (1H, d, J=8.0 Hz), 8.18 (1H, d, J=8.0 Hz), 7.73 (1H, ddd, J=8.2, 7.2.10 Hz), 7.59 (1H, ddd, J=8.2, 7.2, 10, Hz); ¹³C NMR (125 MHz, CDCl₃) δ: 159.9, 146.5, 130.7, 129.9, 127.0, 120.4, 114.4; IR νₘₐₓ (film)/cm⁻¹ 3103, 1686, 1594; HRMS (EI) calcd for C₆¹³CH₆ON₃ (M+H)+: m/z 148.0467, found m/z 147.0469; m.p. 93-94° C.

### ¹³C—(R)—N-Formyl-2,2,5,5-tetramethyl-1,3-dioxane-4-carboxamide

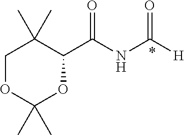

(R)-2,2,5,5-Tetramethyl-1,3-dioxane-4-carboxamide (known from Han et al.) (1.33 g, 7.12 mmol) was dissolved in THF (40 mL ). The solution was cooled to 0° C. and n-butyllithium (3.13 mL , 2.5 M in hexanes, 7.83 mmol) was added slowly via syringe before leaving to stir at 0° C. for 0.5 h. A solution of ¹³C-1H-benzotriazole-1-carboxaldehyde (1.16 g, 7.83 mmol) in THF (10 mL ) was added slowly via syringe. The reaction mixture was left to stir for 4 h at RT. The reaction mixture was diluted with isopropanol (10 mL ) before washing with aqueous saturated NaHCO₃ (50 mL ). The aqueous was separated and extracted with EtOAc (3×50 mL ) before the combined organics were washed with brine (100 mL ), dried (Na₂SO₄), filtered and concentrated in vacuo to give the crude product as a pale yellow solid. The crude residue was purified by flash column chromatography (10-20% EtOAc/petroleum ether) gave the product imide (1.20 g, 78%) as a white solid.

¹H NMR (CDCl₃, 500 MHz) δ: 9.38-8.96 (1H, dd, J=193.5, 10.4 Hz), 8.92 (1H, brs), 4.21 (1H, s), 3.73 (1H, d, J=11.9 Hz), 3.35 (1H, d, J=11.9 Hz), 1.50 (3H, s), 1.47 (3H, s), 1.08 (3H, s), 1.07 (3H, s); ¹³C NMR (CDCl₃, 125 MHz) δ: 170.6, 161.4, 99.7, 77.1, 71.2, 33.4, 29.4, 21.7, 18.9, 18.6; IR νₘₐₓ (film)/cm⁻¹ 3264, 2994, 2960, 2882, 1724, 1657, 1457; HRMS (ESI) calcd for C₉¹³C₁H₁₇NO₄Na M+: m/z 239.1083, found m/z 239.1081; m.p. 130-131° C.

### ¹³C₃—(R,E)-2-(Trimethylsilyl)ethyl 3-(2,2,5,5-tetramethyl-1,3-dioxane-4-carboxamido) acrylate and ¹³C₃—(R,Z)-2-(Trimethylsilyl)ethyl 3-(2,2,5,5-tetramethyl-1,3-dioxane-4-carboxamido)acrylate

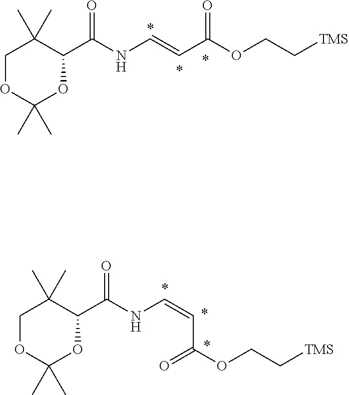 [SMILES: CC1(C)OCC(C)(C)[C@H](C(=O)N/C=C/C(=O)OCC[Si](C)(C)C)O1 ; CC1(C)OCC(C)(C)[C@H](C(=O)N/C=C\C(=O)OCC[Si](C)(C)C)O1]

1,2-¹³C₂-2-(Trimethylsilylethoxycarbonylmethyl) triphenylphosphonium bromide (1.42 g, 2.85 mmol) was dissolved in CH₂Cl₂ (50 mL ) and stirred with aqueous 1 M NaOH (50 mL ) for 2 h at rt. The organic layer was then separated and concentrated in vacuo to give the crude trimethylsilyl ylide (1.24 g) as a yellow oil. The crude ylide was dissolved in benzene (15 mL ) before addition of ¹³C—(R)—N-formyl-2,2,5,5-tetramethyl-1,3-dioxane-4-carboxamide (425 mg, 1.84 mmol). The resulting solution was then stirred at 80° C. for 16 h. The reaction mixture was concentrated in vacuo and subsequent purification of the crude residue by flash column chromatography (5-20% EtOAc/petroleum ether) afforded the cis isomer (99 mg, 15%) as a yellow oil, followed by the trans isomer (537 mg, 82%) as a white solid.

¹³C₃—(R,E)-2-(Trimethylsilyl)Ethyl 3-(2,2,5,5-tetramethyl-1,3-dioxane-4-carboxamido) acrylate: ¹H NMR (CDCl₃, 500 MHz) δ: 8.33 (1H, d, J=11.5 Hz), 8.13-7.71 (1H, dddd, J=177.0, 14.5, 11.5, 5.0 Hz), 5.70-5.34 (1H, dddd, J=163.0, 14.5, 3.0, 1.5 Hz), 4.25-4.21 (2H, m), 4.17 (1H, s), 3.67 (1H, d, J=11.8 Hz), 3.27 (1H, d, J=11.8 Hz), 1.52 (3H, s), 1.46 (3H, s), 1.06 (3H, s), 1.02 (2H, t, J=8.4 Hz), 1.01 (3H, s), 0.05 (9H, s); ¹³C NMR (CDCl₃, 125 MHz) δ: 169.4, 169.0 (dd, J=79.5, 4.3 Hz), 137.5 (dd, J=79.5, 4.3 Hz), 104.7 (dd, J=79.5, 79.5 Hz), 101.0, 78.7, 72.7, 63.8, 34.9, 30.9, 23.3, 20.3, 20.1, 18.8, 0.0; IR νₘₐₓ (film)/cm⁻¹ 3306, 2955, 2899, 2874, 1708, 1649, 1476; HRMS (EI) calcd for C₁₄¹³C₃H₃₂O₅NSi M+: m/z 360.2073, found m/z 360.2064; m.p. 126-127° C.

¹³C₃—(R,Z)-2-(Trimethylsilyl)ethyl 3-(2,2,5,5-tetramethyl-1,3-dioxane-4-carboxamido) acrylate: ¹H NMR (CDCl₃, 400 MHz) δ: 11.10 (1H, d, J=11.6 Hz), 7.62-7.09 (1H, dm, J=175.2 Hz), 5.32-4.86 (1H, ddm, J=168.8, 9.2 Hz), 4.23 (2H, tm, J=8.3 Hz), 4.21 (1H, s), 3.72 (1H, d, J=11.6 Hz), 3.32 (1H, d, J=11.6 Hz), 1.59 (3H, s), 1.47 (3H, s), 1.05 (3H, s), 1.04 (3H, s), 1.04-1.00 (2H, m), 0.05 (9H, s); ¹³C NMR (CDCl₃, 100 MHz) δ: 170.2, 169.8 (d, J=75.4 Hz), 137.3 (d, J=74.4 Hz), 100.8 (dd, J=75.4, 74.4 Hz), 99.7, 78.7, 72.8, 63.7, 34.8, 30.8, 23.4, 20.5, 20.1, 18.9, 0.0; IR νₘₐₓ (film)/cm⁻¹ 3300, 2965, 2924, 2860, 1672, 1630, 1454; HRMS (EI) calcd for C₁₄¹³C₃H₃₂O₅NSi M+: m/z 360.2073, found m/z 360.2064.

### ¹³C₃—(R,E)-3-(2,2,5,5-Tetramethyl-1,3-dioxane-4-carboxamido)acrylic Acid

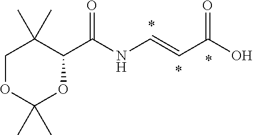 [SMILES: CC1(C)OCC(C)(C)[C@H](C(=O)N/C=C/C(=O)O)O1]

¹³C₃—(R,E)-2-(Trimethylsilyl)ethyl 3-(2,2,5,5-tetramethyl-1,3-dioxane-4-carboxamido) acrylate (500 mg, 1.40 mmol) was dissolved in THF (10 mL ). Water (0.200 mL ) was added before addition of TBAF (4.20 mL , 4.2 mmol, 1M solution in THF) was added via syringe and the resultant pale orange solution was heated at 40° C. for 18 h. The reaction mixture was concentrated in vacuo and the crude residue purified by flash column chromatography (0-2% MeOH/CH₂Cl₂) to yield an impure white solid. The crude product was dissolved in EtOAc (25 mL ) and stirred with aqueous 1 M NaOH (25 mL ) for 1 h. The aqueous layer was collected, diluted with EtOAc (50 mL ) and then acidified to pH 5 with aqueous 1 M HCl. The mixture was allowed to stir for 1 h before extracting the aqueous layer with EtOAc (3×25 mL ). The combined organics were washed with brine (50 mL ), dried (Na₂SO₄), filtered and concentrated in vacuo to afford the desired acid (287 mg, 80%) as a white solid.

¹H NMR (CDCl₃, 500 MHz) δ: 8.52 (1H, d, J=11.8 Hz), 8.26-7.85 (1H, dddd, J=176.5, 14.2, 11.8, 5.0 Hz), 5.76-5.40 (1H, ddm, J=163.0, 14.2 Hz), 4.22 (1H, s), 3.71 (1H, d, J=11.6 Hz), 3.32 (1H, d, J=11.6 Hz), 1.55 (3H, s), 1.51 (3H, s), 1.04 (3H, s), 1.03 (3H, s); ¹³C NMR (CDCl₃, 125 MHz) δ: 172.6, 172.9 (dd, J=77.3, 4.4 Hz), 138.1 (dd, J=77.1, 4.4 Hz), 101.8 (dd, J=77.3, 77.1 Hz), 99.6, 77.2, 71.2, 33.4, 29.4, 21.8, 18.8, 18.7; IR νmax (film)/cm⁻¹ 3315, 3085, 2995, 2880, 1695, 1668; HRMS (EI) calcd for C₉¹³C₃H₂₀O₅N M+: m/z 260.1365, found m/z 260.1364; m.p. 187-188° C.

### ¹³C₃—(R,E)-3-(2,4-Dihydroxy-3,3-dimethylbutanamido)acrylic Acid

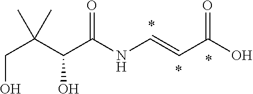

¹³C₃—(R,E)-3-(2,2,5,5-Tetramethyl-1,3-dioxane-4-carboxamido)acrylic acid (100 mg, 0.389 mmol) was dissolved in MeCN (3 mL ). Water (300 μL) was added followed by BiCl₃ (12 mg, 38 μmol), resulting in a white suspension. The mixture was allowed to stir at room temperature for 16 h before filtering through a small Celite® pad that was subsequently washed with EtOAc (20 mL ). Purification of the crude residue by flash column chromatography afforded ¹³C₃-CJ-15,801 as a colourless oil (35 mg, 41%).

¹H NMR ((CD₃)₂CO, 500 MHz) δ: 9.70 (1H, d, J=10.5 Hz), 8.09-7.67 (1H, dddd, J=174.0, 14.5, 10.5, 5.2 Hz), 5.87-5.51 (1H, ddm, J=163.5, 14.5 Hz), 4.00 (1H, s), 3.38 (1H, d, J=10.9 Hz), 3.31 (1H, d, J=10.9 Hz), 0.82 (3H, s), 0.81 (3H, s); ¹³C NMR ((CD₃)₂CO, 125 MHz) δ: 173.1, 170.4 (dd, J=76.7, 4.3 Hz), 138.5 (dd, J=76.7, 4.3 Hz), 102.2 (dd, J=76.7, 76.7 Hz), 77.4, 70.2, 40.3, 21.4, 20.5; IR νmax (film)/cm⁻¹ 3295, 2964, 2836, 2878, 1642, 1584; HRMS (ESI) calcd for C₆¹³C₃H₁₅NO₅Na (M+Na)+: m/z 243.0943, found m/z 243.0940.

### ¹³C₃—(R,E)-Benzyl 3-(2,4-dihydroxy-3,3-dimethylbutanamido)acrylate

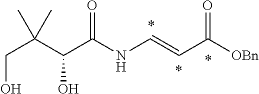 [SMILES: CC(C)(CO)[C@@H](O)C(=O)N/C=C/C(=O)OCc1ccccc1]

A solution of ¹³C₃—(RE)-3-(2,2,5,5-tetramethyl-1,3-dioxane-4-carboxamido)acrylic acid (100 mg, 0.389 mmol) in CH₂Cl₂ (3 mL ) was cooled to 0° C. before sequential addition of benzyl amine (30 μL, 0.291 mmol), catalytic DMAP and DCC (21 mg, 0.101 mmol). The resultant solution was stirred for 4 h at 0° C., by which time a white precipitate had formed. The reaction mixture was filtered through a pad of Celite® that was then washed with EtOAc (20 mL ). The organic layer was then washed with saturated aqueous NaHCO₃ solution (15 mL ), H₂O (15 mL ) and brine (15 mL ) before being dried (Na₂SO₄), filtered and concentrated in vacuo to afford the crude product as white solid (71 mg, 0.203 mmol). After unsuccessful purification of the crude residue by flash column chromatography, the crude product was dissolved in a mixture of MeCN (1.5 mL ) and H₂O (0.150 mL ) before addition of BiCl₃ (6 mg, 19 μmol) to give a milky suspension that was then stirred for 16 h at rt. The reaction mixture was quenched with a few drops of saturated aqueous NaHCO₃ followed by filtration through a pad of Celite® that was subsequently washed with EtOAc (10 mL ). The eluant was concentrated in vacuo to give the crude product as a colourless oil. Purification of the crude residue by flash column chromatography (20-70% EtOAc/petroleum ether) gave the diol product (26 mg, 41%) as a colourless oil.

¹H NMR (CDCl₃, 400 MHz) δ: 9.03 (1H, d, J=11.6 Hz), 8.29-7.77 (1H, dddd, J=176.7, 13.8, 11.6, 4.9 Hz), 7.38-7.34 (5H, m), 5.89-5.41 (1H, ddm, J=163.1, 13.8 Hz), 5.18 (2H, d, J=3.3 Hz), 4.58 (1H, d, J=4.1 Hz), 4.17 (1H, d, J=4.1 Hz), 3.58 (1H, br d, J=10.6 Hz), 3.53 (1H, d, J=10.6 Hz), 3.04 (1H, brs), 1.02 (3H, s), 0.97 (3H, s); ¹³C NMR (CDCl₃, 100 MHz) δ: 171.4 (d, J=3.6 Hz), 167.1 (dd, J=80.0, 4.6 Hz), 136.8 (dd, J=77.9, 4.6 Hz), 135.2, 128.6, 128.2, 128.1, 102.5 (dd, J=80.0, 77.9 Hz), 77.4, 71.6, 66.2, 39.4, 20.9, 20.3; IR νₘₐₓ (film)/cm⁻¹ 3316, 2963, 2934, 2875, 1682, 1649; HRMS (ESI) calcd for C₁₃¹³C₃H₂₁O₅NNa M+: m/z 333.1413, found m/z 333.1407.

### Compound Syntheses—BODIPY Amide Conjugate

### 3-(1H-Pyrrol-2-yl)propan-1-amine

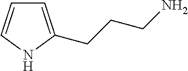

2-(3-Azidopropyl)-1H-pyrrole (100 mg, 0.704 mmol) was dissolved in MeOH (3 mL ), and placed under at atmosphere of argon. Pd/C (10 mg, 10%) was added before replacing the argon with a hydrogen atmosphere before stirring at room temperature for 1.5 h. The crude mixture was filtered over a bed of Celite® to remove Pd, before washing with MeOH (10 mL ) and removing solvent in vacuo to afford the amine product (84 mg, 100%) as a pale yellow oil.

¹H NMR (CDCl₃, 400 MHz) δ: 8.73 (1H, brs), 6.68-6.66 (1H, m), 6.13-6.11 (1H, m), 5.94-5.92 (1H, m), 2.78 (2H, t, J=6.8 Hz), 2.69 (2H, t, J=7.4 Hz), 1.81-1.75 (2H, m), 1.57 (2H, brs); ¹³C NMR (CDCl₃, 100 MHz) δ: 132.1, 116.2, 108.5, 105.0, 41.7, 33.0, 25.2; IR νₘₐₓ (film)/cm⁻¹ 3364, 3233, 3098, 2932, 2851; HRMS (EI) calcd for C₇H₁₃N₂ (M+H)+: m/z 125.1079, found m/z 125.1077.

### (9H-Fluoren-9-yl)methyl 3-(1H-pyrrol-2-yl)propylcarbamate

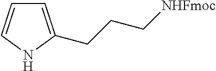

3-(1H-Pyrrol-2-yl)propan-1-amine (719 mg, 5.75 mmol) was dissolved in CH₂Cl₂ (45 mL ) before addition of Et₃N (1.61 mL , 11.5 mmol) and fluorenylmethyloxycarbonyl chloride (1.64 g, 6.33 mmol). The resulting solution was then stirred for 16 h at rt. The reaction mixture was diluted with CH₂Cl₂ (25 mL ) and then washed with aqueous saturated NaHCO₃ (30 mL ), water (30 mL ) and brine (30 mL ), dried (Na₂SO₄) and filtered before being concentrated in vacuo to yield the crude product as a colourless oil. Purification by flash column chromatography (10-20% EtOAc/petroleum ether) afforded the clean Fmoc protected amine (1.15 g, 58%) as a white solid.

¹H NMR (CDCl₃, 400 MHz) δ: 8.58 (1H, br s) 7.79 (2H, d, J=7.3 Hz), 7.61 (2H, d, J=7.3 Hz), 7.42 (2H, t, J=7.3 Hz), 7.32 (2H, t, J=7.4 Hz), 6.71-6.69 (1H, m), 6.13-6.11 (1H, m), 5.93-5.90 (1H, m) 4.79 (1H, br s), 4.47 (2H, d, J=6.7 Hz), 4.23 (1H, t, J=6.7 Hz), 3.30-3.27 (2H, m), 3.05 (2H, t, J=6.8 Hz), 1.80-1.73 (2H, m); ¹³C NMR (CDCl₃, 125 MHz) δ: 157.2, 143.9, 141.4, 131.5, 127.7, 127.1, 125.0, 124.7, 116.6, 108.1, 105.3, 66.6, 47.3, 39.9, 31.0, 24.0; IR νₘₐₓ (film)/cm⁻¹ 3403, 3341,2982, 2928, 1690; HRMS (Cl) calcd for C₂₂H₂₂N₂O₂ (M)+: m/z 346.1681, found m/z 346.1676.

### 3-[(9H-Fluoren-9-ylmethoxy)carbonyl]amino-[4,4-difluoro-5,7-dimethyl-4-bora-3a,4a-diaza-s-indacene-3-yl]propane

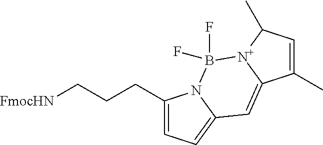

(9H-Fluoren-9-yl)methyl-3-(1H-pyrrol-2-yl)propylcarbamate (692 mg, 0.739 mmol) and 3,5-dimethyl-1H-pyrrole-2-carbaldehyde (268 mg, 2.20 mmol) were dissolved in CH₂Cl₂ (15 mL ) and cooled to 0° C. before dropwise addition of POCl₃ (0.205 mL , 2.20 mmol). The mixture was stirred for 6 h at room temperature before cooling it down again to 0° C. and adding BF₃.Et₂O (0.987 mL , 8.00 mmol) and DIPEA (1.46 mL , 8.40 mmol) and leaving to stir at room temperature for 12 h. The mixture was then diluted with H₂O (25 mL ) and CH₂Cl₂ (10 mL ) before being filtered through a bed of Celite® and washed through with CH₂Cl₂ (2×25 mL ). The combined organics were then dried (Na₂SO₄) and concentrated in vacuo to afford the crude product as a dark red/green solid. Purification of the crude residue by flash column chromatography (10-40% EtOAc/petroleum ether) afforded the product (320 mg, 32%) as a red oil which solidified upon cooling. The NMR values are comparable with a similar BODIPY compound (see Gießler et al.).

¹H NMR (CDCl₃, 400 MHz) δ: 7.77 (2H, d, J=7.3 Hz), 7.62 (2H, d, J=7.3 Hz), 7.40 (2H, t, J=7.3 Hz), 7.30 (2H, t, J=7.4 Hz), 7.08 (1H, s), 6.92 (1H, d, J=3.9 Hz), 6.32 (1H, d, J=3.9 Hz), 6.13 (1H, s), 5.26 (1H, brs), 4.36 (2H, d, J=7.3 Hz), 4.22 (1H, t, J=7.3 Hz), 3.30-3.27 (2H, m), 3.05 (2H, t, J=7.1 Hz), 2.59 (3H, s), 2.26 (3H, s), 2.01-1.95 (2H, m); ¹³C NMR (CDCl₃, 125 MHz) δ: 159.7, 158.9, 156.4, 144.1, 143.5, 141.3, 134.9, 133.2, 128.5, 127.6, 127.0, 125.2, 123.7, 120.2, 119.9, 116.9, 66.6, 47.3, 40.2, 29.0, 25.6, 14.9, 11.3; IR νₘₐₓ (film)/cm⁻¹ 3333, 2943, 2859, 1601.

### 3-(1H-Pyrrol-2-yl)propan-1-ol

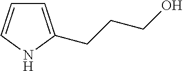

A solution of methyl 3-(1H-pyrrol-2-yl)propanoate (2.64 g, 17.5 mmol) in Et₂O (130 mL ) was cooled to 0° C. before slow addition of LiAlH₄ (996 mg, 26.3 mmol). The suspension was left to stir for 16 h coming to rt. The crude reaction mixture was quenched with 1M NaOH dropwise until pH neutral. Et₂O was decanted off, and the lithium/aluminium salts were washed with further Et₂O (3×50 mL ). The combined organics were dried (Na₂SO₄), filtered and solvent removed in vacuo to give the alcohol product (2.15 g, 100%) as a colourless oil.

¹H NMR (CDCl₃, 400 MHz) δ: 8.24 (1H, brs), 6.70-6.68 (1H, m), 6.15-6.13 (1H, m), 5.95-5.94 (1H, m), 3.73 (2H, t, J=5.9 Hz), 2.75 (2H, t, J=7.3 Hz), 1.93-1.87 (2H, m), 1.50 (1H, brs); ¹³C NMR (CDCl₃, 100 MHz) δ: 131.8, 116.4, 108.3, 105.2, 62.3, 32.2, 24.2; IR νₘₐₓ (film)/cm⁻¹ 3365, 2940, 2976, 2850, 1012; HRMS (Cl) calcd for C₇H₁₂NO (M+H)+: m/z 126.0919, found m/z 126.0917.

### 2-(3-Azidopropyl)-1H-pyrrole

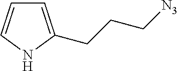 [SMILES: [N-]=[N+]=NCCCc1ccc[nH]1]

A solution of 3-(1H-pyrrol-2-yl)propan-1-ol (1.00 g, 8.13 mmol) in CH₂Cl₂ (60 mL ) was cooled to 0° C. before addition of Et3N (2.26 mL , 16.26 mmol) and methanesulfonyl chloride (0.755 mL , 9.76 mmol). The reaction mixture was allowed to stir for 1 h at 0° C. before being warmed to room temperature and washed with 1 M HCl (40 mL ), aqueous saturated NaHCO₃ (60 mL ) and brine (60 mL ), before being dried (Na₂SO₄), filtered and solvent removed in vacuo to afford the mesylate intermediate (1.52 g, 94%). The crude mesylate was then dissolved in DMF (60 mL ) and the solution was treated with sodium azide (1.48 g, 22.7 mmol) was added before heating to 70° C. for 16 h. The reaction mixture was then cooled to room temperature before adding EtOAc (60 mL ) followed by H₂O (60 mL ). The aqueous phase was washed with EtOAc (2×60 mL ), and then combined organics were then washed with brine (5×100 mL ), dried (Na₂SO₄), filtered and solvent removed in vacuo to afford the azide product (947 mg, 88%) as a yellow oil.

¹H NMR (CDCl₃, 400 MHz) δ: 7.98 (1H, brs), 6.70-6.68 (1H, m), 6.15-6.13 (1H, m), 5.96-5.94 (1H, m), 3.34 (2H, t, J=6.6 Hz), 2.73 (2H, t, J=7.4 Hz), 1.92-1.87 (2H, m); ¹³C NMR (CDCl₃, 100 MHz) δ: 130.7, 116.5, 108.5, 105.5, 50.7, 28.9, 24.7. IR νₘₐₓ (film)/cm⁻¹ 3379, 2940, 2870, 2091; HRMS (EI) calcd for C₇H₁₁N₄ (M+H)+: m/z 151.0984, found m/z 151.0987.

### 3-Azido[4,4-difluoro-5,7-dimethyl-4-bora-3a,4a-diaza-s-indacene-3-yl]propane, 11

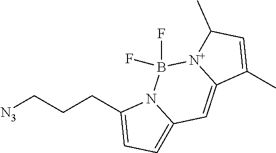

2-(3-Azidopropyl)-1H-pyrrole (105 mg, 0.739 mmol) and 3,5-dimethyl-1H-pyrrole-2-carboxaldehyde (99 mg, 0.813 mmol) were dissolved in CH₂Cl₂ (5 mL ) and cooled to 0° C. before dropwise addition of POCl₃ (76 μL, 0.813 mmol). The mixture was allowed to stir for 6 h at room temperature before being cooled again to 0° C. and adding BF₃.Et₂O (0.365 mL , 2.96 mmol) and DIPEA (0.539 mL , 3.10 mmol) and leaving to stir at room temperature for 12 h. The mixture was then diluted with H₂O (10 mL ) and CH₂Cl₂ (5 mL ) before being filtered through a bed of Celite® and washed through with CH₂Cl₂ (2×10 mL ). The combined organics were then dried (Na₂SO₄) and solvent removed in vacuo to afford the crude as a dark red/green solid. Purification of the crude residue by flash column chromatography (5% EtOAc/petroleum ether) afforded the desired azide (155 mg, 57%) as a red oil which solidified upon cooling. The NMR values are comparable with a similar BODIPY compound (see Gießler et al.).

¹H NMR (CDCl₃, 500 MHz) δ: 7.09 (1H, s), 6.91 (1H, d, J=3.9 Hz), 6.28 (1H, d, J=3.9 Hz), 6.12 (1H, s), 3.40 (2H, t, J=7.0 Hz), 3.05 (2H, t, J=7.4 Hz), 2.57 (3H, s), 2.27 (3H, s), 2.04 (2H, m); ¹³C NMR (CDCl₃, 125 MHz) δ: 160.3, 157.8, 147.9, 143.7, 133.3, 128.2, 123.7, 120.4, 116.6, 50.9, 28.1, 25.8, 14.9, 11.3. IR νmax (film)/cm⁻¹ 2959, 2932, 2870, 2091; HRMS (ESI) calcd for C₁₄H₁₇BF₂N₅ (M+H)+: m/z 304.1545, found m/z 304.1528; m.p. 49-50° C.

### 3- Amino[4,4-difluoro-5,7-dimethyl-4-bora-3a,4a-diaza-s-indacene-3-yl]propane

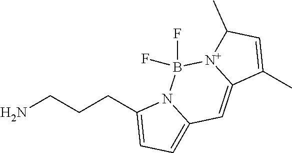

To a solution of 3-azido[4,4-difluoro-5,7-dimethyl-4-bora-3a,4a-diaza-s-indacene-3yl]propane (25 mg, 0.083 mmol) in THF (1 mL ) was added polymer bound triphenylphosphine (103 mg, 1.6 mmolg⁻¹) and H₂O (50 μL). The resultant suspension was heated at 50° C. for 4 h, following reaction progress by TLC. Upon completion, the suspension was allowed to cool to room temperature before filtering through a bed of Celite® and removing solvent in vacuo to afford the desired amine as a red oil (61-82%). The material was used without any further purification.

### Amide Conjugate -310

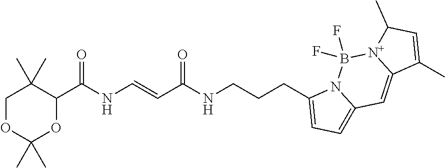

The compound was prepared by amide coupling of 3-amino[4,4-difluoro-5,7-dimethyl-4-bora-3a,4a-diaza-s-indacene-3-yl]propane, prepared in situ from 3-[(9H-Fluoren-9-ylmethoxy)carbonyl]amino-[4,4-difluoro-5,7-dimethyl-4-bora-3a,4a-diaza-s-indacene-3-yl]propane by treatment with piperidine, with (E)-3-(2,2,5,5-tetramethyl-1,3-dioxane-4-carboxamido)acrylic acid.

Thus, 3-[(9H-Fluoren-9-ylmethoxy)carbonyl]amino-[4,4-difluoro-5,7-dimethyl-4-bora-3a,4a-diaza-s-indacene-3-yl]propane was treated with piperidine in THF for 3 h. at room temperature. The crude product was reacted with (E)-3-(2,2,5,5-tetramethyl-1,3-dioxane-4-carboxamido)acrylic acid in the presence of HBTU, DIPEA, CH₂Cl₂ under microwave conditions at 80° C. for 2.5 h. HPLC purification gave crude product in <10% yield over the two steps.

### Compound Syntheses—BODIPY Click Conjugate

### (R,E)-N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide, 1

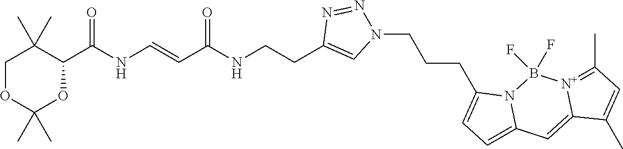

(R,E)-N-(3-(But-3-ynylamino)-3-oxoprop-1-enyl)-2,2,5,5-tetramethyl-1,3-dioxane-4-carboxamide (20 mg, 65 μmol) and 3-azido[4,4-difluoro-5,7-dimethyl-4-bora-3a,4a-diaza-s-indacene-3-yl]propane (15 mg, 50 μmol) were dissolved in THF (2 mL ). One drop of DIPEA was added via pipette, followed by a catalytic amount of CuI. The resultant mixture was heated to 70° C. for 18 h then concentrated in vacuo giving the crude product as a brown oil. Purification of the crude residue by flash column chromatography (0-2% MeOH/CH₂Cl₂) to afford the desired compound (23 mg, 76%) as a red oil.

¹H NMR (CDCl₃, 500 MHz) δ: 8.32 (1H, d, J=11.4 Hz), 7.84 (1H, dd, J=13.9, 11.4 Hz), 7.38 (1H, s), 7.09 (1H, s), 6.87 (1H, d, J=4.1 Hz), 6.34 (1H, brs), 6.23 (1H, d, J=4.1 Hz), 6.11 (1H, s), 5.73 (1H, d, J=13.9 Hz), 4.40 (2H, t, J=6.9 Hz), 4.15 (1H, s), 3.68 (1H, d, J=11.6 Hz), 3.65-3.61 (2H, m), 3.28 (1H, d, J=11.6 Hz), 2.97 (2H, t, J=7.5 Hz), 2.90 (2H, t, J=6.3 Hz), 2.53 (3H, s), 2.38-2.31 (2H, m), 2.24 (3H, s), 1.46 (3H, s), 1.42 (3H, s), 1.02 (3H, s), 0.97 (3H, s); ¹³C NMR (CDCl₃, 125 MHz) δ: 168.0, 166.3, 160.5, 156.6, 144.2, 135.3, 133.1, 132.8, 128.2, 123.9, 122.1, 120.6, 116.6, 106.0, 99.4, 77.2, 71.3, 49.8, 38.7, 33.3, 29.4, 29.3, 25.6, 25.5, 21.9, 18.8, 18.7, 14.9, 11.3; IR νmax (film)/cm⁻¹ 3295, 2991,2830, 2876, 1666, 1598; HRMS (ESI) calcd for C₃₀H₃₉BF₂N₇O₄ (M−H)+: m/z 609.3166, found m/z 609.3141; [α]_{D} +29.6 (c=0.6, (CH₃)₂CO, T=24.4° C.).

The (S,E) form (3) may be prepared in a similar manner from (S,E)-N-(3-(but-3-ynylamino)-3-oxoprop-1-enyl)-2,2,5,5-tetramethyl-1,3-dioxane-4-carboxamide. [α]_{D} −21.8 (c=1.2, (CH₃)₂CO, T=24.4° C.).

### (R,E)-N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,4-dihydroxy-3,3-dimethylbutanamide, 5

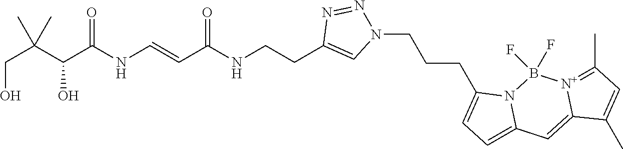

(R,E)-N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide (20 mg, 33 μmol) was dissolved in a mixture of THF (1 mL ) and H₂O (50 μL) before adding BiCl₃ (1 mg, 3 μmol) and leaving to stir at room temperature for 16 h. The crude mixture was filtered through Celite® before concentrating in vacuo. Purification of the crude residue by flash column chromatography (5-8% MeOH/CH₂Cl₂) gave the diol product (10 mg, 56%) as a red solid.

¹H NMR (CDCl₃, 500 MHz) δ: 9.43 (1H, d, J=11.2 Hz), 7.90-7.85 (1H, m), 7.47 (1H, s), 7.09 (2H, brs), 6.86 (1H, d, J=3.9 Hz), 6.22 (1H, d, J=3.9 Hz), 6.10 (1H, s), 5.81 (1H, d, J=13.8 Hz), 5.53 (1H, brs), 4.37-4.34 (3H, m), 4.17 (1H, s), 3.56-3.50 (3H, m), 3.43 (1H, d, J=10.7 Hz), 2.93 (2H, t, J=7.5 Hz), 2.90 (2H, br s), 2.52 (3H, s), 2.31-2.28 (2H, m), 2.22 (3H, s), 0.93 (3H, s), 0.92 (3H, s); ¹³C NMR (CDCl₃, 125 MHz) δ: 172.7, 167.3, 160.5, 156.6, 145.2, 144.2, 135.2, 133.8, 133.2, 128.3, 124.0, 122.1, 120.6, 116.6, 105.6, 76.8, 70.3, 49.7, 39.6, 39.1, 29.3, 25.6, 25.5, 21.0, 20.7, 14.9, 11.3; IR νₘₐₓ (film)/cm⁻¹ 3295, 2970, 2829, 2873, 1661, 1597; HRMS (ESI) calcd for C₂₇H₃₅BF₂N₇O₄ (M−H)+: m/z 569.2853, found m/z 569.2831; m.p. 104-105° C.; [α]_{D} +28.1 (C=0.5, (CH₃)₂CO, T=24.4° C.).

The (S,E) form (7) may be prepared in a similar manner from (S,E)-N-((((3-[4,4-difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide. [α]_{D} −24.0 (c=1.0, (CH₃)₂CO, T=24.4° C.).

### (Z)—N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide, (R)-2, (S)-4

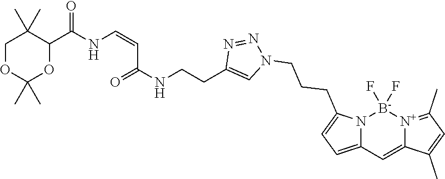 [SMILES: CC1=CC(C)=[N+]2C1=Cc1ccc(CCCn3cc(CCNC(=O)/C=C\NC(=O)C4OC(C)(C)OCC4(C)C)nn3)n1[B-]2(F)F]

(Z)—N-(3-(But-3-yn-1-ylamino)-3-oxoprop-1-en-1-yl)-2,2,5,5-tetramethyl-1,3-dioxane-4-carboxamide (R-form or S-form) (96 mg, 0.3 mmol) was dissolved in THF (1.5 mL ) before 3-azido[4,4-difluoro-5,7-dimethyl-4-bora-3a,4a-diaza-s-indacene-3-yl]propane (122 mg, 0.4 mmol) was added in one portion followed by DIPEA (1 drop) and catalytic amounts of CuI. The resulting mixture was heated to 70° C. for 16 h then concentrated in vacuo to give the crude as a dark brown oil. The crude was purified via column chromatography (5% MeOH/CH₂Cl₂) to give the triazole product (161 mg, 85%) as a red solid.

¹H NMR (CDCl₃, 500 MHz) δ: 11.68 (1H, d, J=11.1 Hz), 7.39 (1H, s), 7.23 (1H, dd, J=11.1, 8.9 Hz), 7.10 (1H, s), 6.88 (1H, d, J=3.9 Hz), 6.35 (1H, t, J=5.5 Hz), 6.24 (1H, d, J=3.9 Hz), 6.12 (1H, s), 5.03 (1H, d, J=8.9 Hz), 4.42 (2H, t, J=6.9 Hz), 4.19 (1H, s), 3.71 (1H, d, J=11.7 Hz), 3.68-3.58 (2H, m), 3.32 (1H, d, J=11.7 Hz), 2.97 (2H, t, J=7.5 Hz), 2.92 (2H, t, J=6.2 Hz), 2.54 (3H, s), 2.37 (2H, quin, J=7.2 Hz), 2.26 (3H, s), 1.60 (3H, s), 1.46 (3H, s), 1.04 (6H, s); ¹³C NMR (CDCl₃, 125 MHz) δ: 168.7, 167.8, 160.6, 156.5, 145.4, 144.2, 135.3, 133.1, 133.0, 128.1, 123.8, 121.7, 120.6, 116.5, 100.8, 99.2, 77.2, 71.4, 49.6, 38.2, 33.3, 29.4, 29.2, 25.5, 25.4, 22.0, 19.0, 18.6, 14.9, 11.3; IR νₘₐₓ (film)/cm⁻¹ (neat): 2926, 2360, 1660, 1610, 1139; HRMS (ESI) calcd for C₃₀H₄₀N₇BF₂O₄Na (M+Na)⁺: m/z 633.3131, found m/z 633.3107; MP Range: 66-69° C.

R-form: [α]_{D} +3.8 (C=0.9, (CH₃)₂CO, T=24.4° C.).

S-form: [α]_{D} −1.6 (c=1.0, (CH₃)₂CO, T=24.4° C.).

### (Z)—N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,4-dihydroxy-3,3-dimethylbutanamide, (R)-6, (S)-8

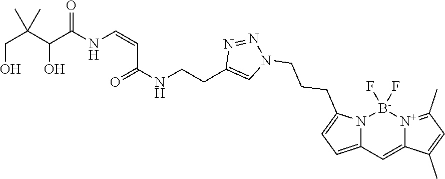 [SMILES: CC1=CC(C)=[N+]2C1=Cc1ccc(CCCn3cc(CCNC(=O)/C=C\NC(=O)C(O)C(C)(C)CO)nn3)n1[B-]2(F)F]

(Z)—N-((((3-[4,4-Difluoro-5,7-dimethyl-4-bora-3a,4a-diaza-s-indacene-3-yl]propyl)-1H-1,2,3-triazol-4-yl)ethylamino)-3-oxoprop-1-enyl)-2,2,5,5-tetramethyl-1,3-dioxane-4-carboxamide (60 mg, 0.1 mmol) was dissolved in MeCN (1.2 mL ) and H₂O (0.1 mL ) before BiCl₃ (6 mg, 0.02 mmol) was added. The mixture was stirred vigorously for 16 h. before filtering through Celite® and washing through with MeCN (40 mL ). Removal of solvent in vacuo to give the crude diol as a dark brown oil. The crude was purified by flash column chromatography (5% MeOH/CH₂Cl₂) to give the diol as a red oil (52 mg, 92%).

¹H NMR (CDCl₃, 400 MHz) δ: 11.85 (1H, d, J=11.1 Hz), 7.42 (1H, s), 7.23 (1H, dd, J=11.1, 8.8 Hz), 7.10 (1H, s), 6.89 (1H, d, J=4.0 Hz), 6.55 (1H, t, J=5.5 Hz), 6.24 (1H, d, J=4.0, Hz), 6.13 (1H, s), 5.05 (1H, d, J=8.8 Hz), 4.42 (2H, t, J=6.9 Hz), 4.16 (1H, s), 3.66-3.55 (2H, m), 3.53 (1H, s), 2.96 (2H, t, J=7.5 Hz), 2.92 (2H, t, J=6.2 Hz), 2.54 (3H, s), 2.35 (2H, quin, J=7.4 Hz), 2.26 (3H, s), 1.03 (3H, s), 0.98 (3H, s); ¹³C NMR (CDCl₃, 125 MHz) δ: 172.0, 168.1, 160.6, 156.5, 145.2, 144.2, 135.3, 133.5, 133.1, 128.2, 123.9, 121.8, 120.6, 116.5, 100.6, 77.9, 71.1, 49.7, 39.4, 38.4, 29.2, 25.5, 25.4, 20.9, 20.6, 14.9, 11.3; IR νₘₐₓ (film)/cm⁻¹ (neat): 3309, 2953, 1654, 1610, 1139; HRMS (ESI) calcd for C₂₇H₃₆N₇BF₂O₄Na (M+Na)⁺: m/z 593.2818, found m/z 593.2810.

R-form: [α]_{D} +15.3 (C=0.6, (CH₃)₂CO, T=24.4° C.).

S-form: [α]_{D} −16.0 (c=1.0, (CH₃)₂CO, T=24.4° C.).

### 7-(3-Azidopropyl)-5,5-difluoro-1-(3-methoxy-3-oxopropyl)-5H-dipyrrolo[1,2-c:1′,2′-F][1,3,2]diazaborinin-4-ium-5-uide

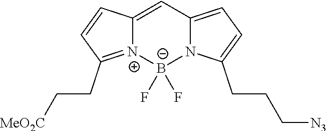 [SMILES: COC(=O)CCC1=[N+]2C(=Cc3ccc(CCCN=[N+]=[N-])n3[B-]2(F)F)C=C1]

A 0° C. solution of 2-(3-azidopropyl)-1H-pyrrole (275 mg, 1.83 mmol) in CH₂Cl₂ (10 mL ) was treated with methyl 3-(5-formyl-7H-pyrrol-2-y/)propanoate (365 mg, 2.01 mmol) in CH₂Cl₂ (10 mL ) and the resulting solution was treated by the dropwise addition of POCl₃ (0.2 mL , 2.01 mmol). The reaction mixture was stirred for 6 h at r.t. before being cooled back down to 0° C. The mixture was then treated with BF₃(OEt)₂ (1 mL , 7.3 mmol) and N,N-diisopropylethylamine (1.4 mL, 8.24 mmol), and the reaction was stirred at r.t. overnight. The mixture was then diluted with H₂O (15 mL) and CH₂Cl₂ (10 mL) before being filtered through a bed of Celite®. The Celite® was washed through with CH₂Cl₂ (2×15 mL) and the organic phases combined. The solution was then dried over Na₂SO₄ and concentrated in vacuo to afford a dark red/green crude solid residue. Purification of the crude solid by flash column chromatography (Pet. Ether:EtOAc; 8:2) afforded 271 mg (41%) of the desired ester as red oil, which solidified upon cooling.

¹H NMR (CDCl₃, 400 MHz) δ: 7.13 (1H, s), 7.03-6.95 (1H, m), 6.35 (1H, t, J=3.7 Hz), 3.70 (1H, s), 3.40 (1H, t, J=6.9 Hz), 3.32 (1H, t, J=7.5 Hz), 3.07 (1H, t, J=7.7 Hz), 2.78 (1H, t, J=7.6 Hz), 2.11-1.96 (1H, m).

### (R)-3-(2,2,5,5-Tetramethyl-1,3-dioxane-4-carboxamido)propanoic Acid

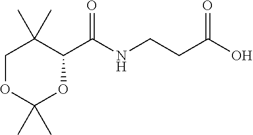 [SMILES: CC1(C)OCC(C)(C)[C@H](C(=O)NCCC(=O)O)O1]

A solution of D-pantothenic acid hemicalcium salt (500 mg, 1.0 mmol) in acetone (25 mL) was treated with p-TsOH.H₂O (560 mg, 3.0 mmol) and 1.0 g of 4 Å molecular sieves. The reaction was stirred at room temperature until completion by TLC analysis (18 h). The suspension was then filtered through a Celite® bed and washed with acetone (2×15 mL), and the organic phases combined. The combined organic washes were concentrated in vacuo before addition of EtOAc (30 mL) to the crude residue. The resulted solution was then washed with brine (2×30 mL). The organic layer was dried over Na₂SO₄, and concentrated under vacuum. Before the complete removal of the solvent, hexane was added dropwise until crystallization of the acetonide was induced (300 mg, 55%). The desired acetonide was obtained as white crystals, which required no further purification.

¹H NMR (CDCl₃, 400 MHz) δ: 7.04 (1H, m), 4.12 (1H, s), 3.71 (1H, d, J=11.6 Hz), 3.66-3.47 (2H, m), 3.30 (1H, d, J=11.6 Hz), 2.65 (2H, t, J=6.0 Hz), 1.48 (3H, s), 1.45 (3H, s), 1.06 (3H, s), 1.00 (3H, s). ¹³C NMR (CDCl₃, 100 MHz) δ: 174.7, 170.1, 99.0, 77.2, 71.4, 34.1, 33.7, 32.9, 29.4, 22.0, 18.8, 18.7.

### (R)—N-(3-(But-3-yn-1-ylamino)-3-oxopropyl)-2,2,5,5-tetramethyl-1,3-dioxane-4-carboxamide

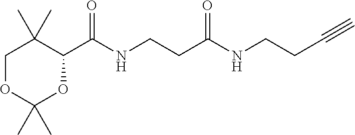 [SMILES: C#CCCNC(=O)CCNC(=O)[C@@H]1OC(C)(C)OCC1(C)C]

A solution of (R)-3-(2,2,5,5-tetramethyl-1,3-dioxane-4-carboxamido)propanoic acid (160 mg, 0.6 mmol) in CH₂Cl₂ (1.5 mL) was treated with HBTU (340 mg, 0.9 mmol) followed by 1-amino-3-butyne (70 μL, 0.9 mmol) and N,N-diisopropylethylamine (150 μL, 0.9 mmol). The reaction mixture was then heated at 80° C. for 3.5 h in the microwave oven. The reaction was then concentrated under reduced pressure. Purification of the crude residue by flash column chromatography (0-5% MeOH/CH₂Cl₂) gave the desired alkyne as a red oil (175 mg, 90%).

¹H NMR (CDCl₃, 400 MHz) δ: 7.05 (1H, brs), 6.24 (1H, br s), 4.10 (1H, s), 3.71 (1H, d, J=12.0 Hz), 3.65-3.50 (2H, m), 3.30 (1H, d, J=12.0 Hz), 3.24-3.17 (2H, m), 2.50 (2H, t, J=8.0 Hz), 2.42 (2H, td, J=6.4, 2.4 Hz), 2.03 (1H, t, J=2.4 Hz), 1.48 (3H, s), 1.46 (3H, s), 1.06 (3H, s), 0.99 (3H, s). ¹³C NMR (CD₃)₂CO, 125 MHz) δ: 171.6, 169.8, 99.4, 82.5, 77.7, 71.8, 70.8, 39.0, 36.1, 35.5, 33.4, 22.3, 19.8, 19.2, 19.0. IR uₘₐₓ (film)/cm⁻¹: 3293, 2989, 1740, 1650, 1536, 1368, 1232, 1090. [α]_{D} +9.50 (c=0.5, CHCl₃, T=23.0° C.).

### (R)—N-((((3-[4,4-Difluoro-5,7-dimethyl-4-bora-3a,4a-diaza-s-indacene-3-yl]propyl)-1H-1,2,3-triazol-4-yl)ethylamino)-3-oxopropyl)-2,2,5,5-tetramethyl-1,3-dioxane-4-carboxamide, 10

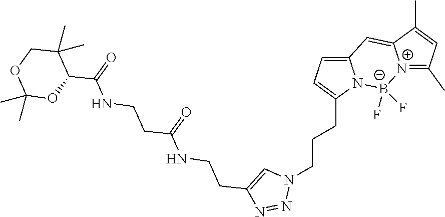

A solution of 3-azido[4,4-difluoro-5,7-dimethyl-4-bora-3a,4a-diaza-s-indacene-3-yl]propane (40 mg, 0.1 mmol) in THF (2.5 mL ) was treated with a solution of N-(3-(but-3-yn-1-ylamino)-3-oxopropyl)-2,2,5,5-tetramethyl-1,3-dioxane-4-carboxamide (43 mg, 0.1 mmol) in THF (2.5 mL ). The resulting mixture was treated with a catalytic amount of CuI (5 mg) followed by N,N-diisopropylethylamine (0.1 mL , 0.6 mmol). The reaction was then heated to 70° C. until completion by TLC analysis (18 h). The reaction mixture was cooled down to room temperature and diluted with H₂O (5 mL ). The mixture was extracted with EtOAc (3×10 mL ) and the combined organics were washed with brine (3×30 mL ), dried over MgSO₄ and concentrated under reduced pressure. Purification of the crude residue by flash column chromatography (0-5% MeOH/CH₂Cl₂) afforded the expected triazole 10 as a red oil (74 mg, 87%).

¹H NMR (CDCl₃, 400 MHz) δ: 7.45 (1H, s), 7.13 (1H, s), 6.91 (1H, d, J=4.0 Hz), 6.61 (1H, t, J=5.0 Hz), 6.28 (1H, d, J=4.0 Hz), 6.15 (1H, s), 4.44 (2H, t, J=8.0 Hz), 4.07 (1H, s), 3.70 (1H, d, J=12.0 Hz), 3.62-3.56 (3H, m), 3.54-3.45 (1H, m), 3.28 (1H, d, J=12.0 Hz), 3.01 (2H, t, J=8.0 Hz), 2.90 (2H, t, J=6.4 Hz), 2.56 (3H, s), 2.43 (2H, t, J=6.0 Hz), 2.39-2.33 (2H, m), 2.28 (3H, s), 1.46 (3H, s), 1.44 (3H, s), 1.03 (3H, s), 0.96 (3H, s). ¹³C NMR (CDCl₃, 100 MHz) δ: 171.2, 169.9, 160.5, 156.5, 145.2, 135.0, 133.1, 132.0, 128.8, 123.9, 121.9, 120.6, 116.5, 99.1, 77.1, 71.4, 49.7, 38.7, 35.9, 34.8, 32.9, 29.4, 29.3, 25.8, 25.4, 22.1, 18.9, 18.6, 14.9, 11.3. ¹⁹F NMR (CDCl₃, 376 MHz) δ: −70.1, −72.0. IR uₘₐₓ (film)/cm⁻¹: 3357, 2922, 2850, 1740, 1650. [α]_{D} −15.50 (c=0.1, CHCl₃, T=23.0° C.).

### (R)—N-((((3-[4,4-Difluoro-5,7-dimethyl-4-bora-3a,4a-diaza-s-indacene-3-yl]propyl)-1H-1,2,3-triazol-4-yl)ethylamino)-3-oxopropyl)-2,4-dihydroxy-3,3-dimethylbutanamide, 9

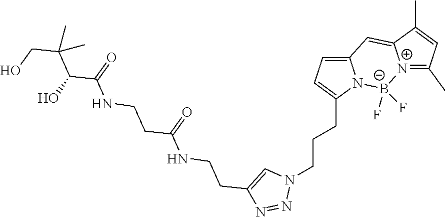 [SMILES: CC1=CC(C)=[N+]2C1=Cc1ccc(CCCn3cc(CCNC(=O)CCNC(=O)[C@H](O)C(C)(C)CO)nn3)n1[B-]2(F)F]

A solution of (R)—N-((((3-[4,4-difluoro-5,7-dimethyl-4-bora-3a,4a-diaza-s-indacene-3-yl]propyl)-1H-1,2,3-triazol-4-yl)ethylamino)-3-oxopropyl)-2,2,5,5-tetramethyl-1,3-dioxane-4-carboxamide 10 (40 mg, 60 μmol) in CH₃CN (3 mL ) was treated with a catalytic amount of BiCl₃ (5 mg) followed by H₂O (0.1 mL ). The reaction mixture was then stirred at room temperature until completion by TLC analysis (17 h). The reaction was then quenched by the addition of a few drops of saturated aqueous. NaHCO₃, and diluted with EtOAc (5 mL ). The resulting suspension was filtered through a bed of Celite® which was then washed thoroughly with EtOAc (2×5 mL ). The combined organic washes were dried over Na₂SO₄, and concentrated under reduced pressure. Purification of the crude residue by flash column chromatography (0-10% 2M NH₃ in MeOH/CH₂Cl₂) afforded the expected diol 9 as a red oil (10 mg, 28%).

¹H NMR (CD₃OD, 400 MHz) δ: 8.57 (1H, s), 7.45 (1H, s), 7.06 (1H, s), 7.00 (1H, d, J=4.0 Hz), 6.71 (1H, br s), 6.43 (1H, d, J=4.0 Hz), 6.24 (1H, s), 4.51 (2H, t, J=8.0 Hz), 4.09 (1H, s), 3.69 (1H, d, J=12.0 Hz), 3.63-3.59 (2H, m), 3.56-3.53 (2H, m), 3.15 (1H, d, J=12.0 Hz), 3.00 (2H, t, J=8.0 Hz), 2.92 (2H, t, J=6.4 Hz), 2.51 (3H, s), 2.31 (2H, t, J=6.0 Hz), 2.20-2.12 (2H, m), 2.07 (3H, s), 1.04 (3H, s), 0.74 (3H, s). ¹³C NMR (CD₃OD, 100 MHz) δ: 179.0, 167.8, 160.3, 155.8, 145.4, 134.2, 132.4, 130.8, 129.6, 124.3, 122.5, 120.9, 116.2, 100.5, 77.4, 71.8, 50.1, 38.7, 35.9, 35.1, 31.6, 29.0, 25.5, 18.7, 18.4, 13.0, 11.4. ¹⁹F NMR (CD₃OD, 376 MHz) δ: −74.1, −76.0. IR uₘₐₓ (film)/cm⁻¹: 3491, 2927, 1843, 1644, 1557, 1411, 1258. [α]_{D} −26.00 (c=0.02, CHCl₃, T=23.0° C.).

### 7-(3-Azidopropyl)-3-(2-carboxyethyl)-5,5-difluoro-5H-dipyrrolo[1,2-c:2′,1′-f][1,3,2]diazaborinin-4-ium-5-uide

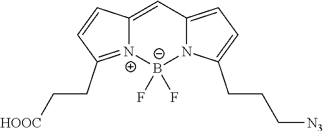 [SMILES: [N-]=[N+]=NCCCc1ccc2n1[B-](F)(F)[N+]1=C(CCC(=O)O)C=CC1=C2]

A solution of 7-(3-azidopropyl)-5,5-difluoro-1-(3-methoxy-3-oxopropyl)-5H-dipyrrolo[1,2-c:1′,2′-F][1,3,2]diazaborinin-4-ium-5-uide (114 mg, 315 μmol) in THF (13 mL ) was treated with H₂O (7 mL ) and concentrated HCl (5 mL ). The mixture was stirred overnight at r.t. and then diluted with H₂O and extracted with CH₂Cl₂. The organic extracts were dried over Na₂SO₄, and concentrated under reduced pressure. The crude product was purified by column chromatography (Pet. Ether:EtOAc; 7:3) obtaining the desired acid as a red solid in 68% yield.

¹H NMR (CDCl₃, 400 MHz) δ: 7.14 (1H, s), 7.01 (2H, dd, J=11.9, 4.1 Hz), 6.36 (2H, d, J=4.1 Hz), 3.40 (2H, t, J=6.9 Hz), 3.32 (2H, t, J=7.5 Hz), 3.07 (2H, t, J=7.7 Hz), 2.84 (2H, t, J=7.5 Hz), 2.08-2.00 (2H, m). ¹³C NMR: (CDCl₃, 100 MHz) δ: 161.56, 160.42, 134.85, 130.79, 130.63, 127.97, 118.56, 51.02, 33.05, 29.84, 28.14, 26.17, 24.06, 14.25. ¹⁹F NMR: (377 MHz, CDCl₃) δ −143.75, −143.84, −143.93, −144.02. IR Vmax (film)/cm⁻¹ 2954, 2925, 2854, 2097,1709,1604,1439,1114.

### (Rac)-Praziquanamine-2,3,6,7-tetrahydro-1H-pyrazino[2,1-a]isoquinolin-4(11bH)-one

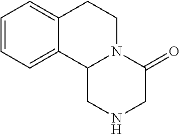 [SMILES: O=C1CNCC2c3ccccc3CCN12]

1 N HCl solution (7.5 mL ) was added to a 25 mL round bottom flask containing praziquantel (250 mg, 0.8 mmol) in 2 mL of EtOH. The mixture was refluxed for 60 h. After that time the reaction was washed with 5 mL of EtOAc at r.t. and cooled down in an ice bath. Then, 5M NaOH was added to adjust the pH to 12-14 (using pH paper). The aqueous layer was extracted with CH₂Cl₂ and the organic layer was dried with Na₂SO₄ and dried in vacuo. The pale yellow solid was purified by column chromatography (CH₂Cl₂:MeOH; 9:1) to afford pure product praziquanamine in 83% yield (135 mg). The NMR data obtained matches the previously reported (PLoS Negl Trap Dis. 2011 September; 5(9): e1260).

¹H NMR (CDCl₃, 400 MHz) δ: 7.25-7.09 (4H, m), 4.90-4.83 (1H, m), 4.80 (1H, dd, J=10.0, 4.5 Hz), 3.73 (1H, dd, J=13.0, 4.7 Hz), 3.60 (2H, dd, J=56.8, 17.4 Hz), 3.05-2.68 (4H, m), 2.00 (1H, s).

### 5,5-Difluoro-1,3-dimethyl-7-(3-oxo-3-(4-oxo-3,4,6,7-tetrahydro-1-pyrazino[2,1-a]isoquinolin 2(11 bH)-yl)propyl)-5H-dipyrrolo[1,2-c:2′,1′-f][1,3,2]diazaborinin-4-ium-5-uide, 12

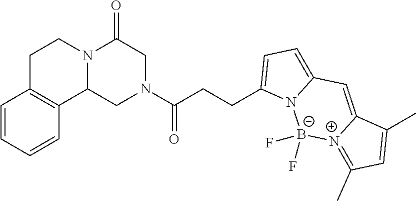 [SMILES: CC1=CC(C)=[N+]2C1=Cc1ccc(CCC(=O)N3CC(=O)N4CCc5ccccc5C4C3)n1[B-]2(F)F]

A solution of BODIPY acid (53 mg, 0.18 mmol) and PZQamine (35 mg, 0.18 mmol) in 1 mL of CH₂Cl₂, was treated with EDC (80 mg, 0.36 mmol) and the reaction was stirred at r.t. overnight. Once the reaction was finished as indicated by TLC (CH₂Cl₂:MeOH; 95:5), the mixture was concentrated in vacuo. The remaining red oil was dissolved in EtOAc and washed with a saturated aqueous. Na₂CO₃ and brine. The organic layer was dried with Na₂SO₄ and concentrated under reduced pressure. The crude residue was purified by column chromatography (CH₂Cl₂:MeOH; 98:2) to yield a red solid in 90% yield (80 mg). NMR data obtained matches the previously reported. Mol. Biochem. Parasitol. 164:57-65, 2009.

¹H NMR (CDCl₃, 400 MHz) δ: 7.28-7.00 (4H, m), 6.88 (1H, t, J=3.7 Hz), 6.38 6.09 (1H, m), 5.19-5.12 (1H, m), 4.95-4.62 (2H, m), 4.54-4.35 (1H, m), 3.90 (1H, dd, J=86.2, 18.0 Hz), 3.34 (1H, t, J=7.5 Hz), 3.15-2.70 (4H, m), 2.59 (2H, d, J=8.3 Hz), 2.26 (2H, s), 1.28 (2H, s).

### 3-(3-Azidopropyl)-5,5-difluoro-7-(3-oxo-3-(4-oxo-3,4,6,7-tetrahydro-1H-pyrazino[2,1-a]isoquinolin-2(11bH)-yl)propyl)-5H-dipyrrolo[1,2-c:2′,1′-f][1,3,2]diazaborinin-4-ium-5-uide

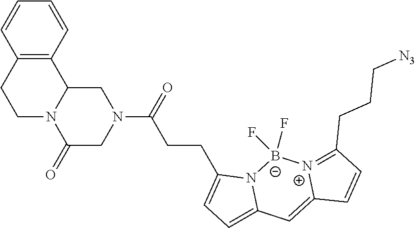 [SMILES: [N-]=[N+]=NCCCC1=[N+]2C(=Cc3ccc(CCC(=O)N4CC(=O)N5CCc6ccccc6C5C4)n3[B-]2(F)F)C=C1]

A solution of bis-functionalized BODIPY acid (40 mg, 0.11 mmol) and PZQamine (23 mg, 0.11 mmol) in 1 mL of CH₂Cl₂, was treated with EDC (55 mg, 0.23 mmol) and the resulting mixture was stirred at r.t. overnight. Once the reaction finished as checked by TLC (CH₂Cl₂:MeOH; 95:5), the mixture was concentrated in vacuo. The remaining red oil was dissolved in EtOAc and washed with saturated aqueous Na₂CO₃ and brine. The organic layer was dried with Na₂SO₄ and concentrated under reduced pressure. The crude residue was purified by flash column chromatography (CH₂Cl₂:MeOH; 97:3) to yield the desired product as a red solid in 90% yield (80 mg).

¹H NMR (CDCl₃, 400 MHz) δ: 7.32-6.96 (4H, m), 6.39 (1H, td, J=11.0, 4.1 Hz), 5.15 (1H, dd, J=13.4, 2.8 Hz), 4.87-4.67 (1H, m), 4.46-4.34 (1H, m), 3.92 (1H, dd, J=83.6, 18.0 Hz), 3.44-3.30 (2H, m), 3.18-2.69 (4H, m), 2.11-1.96 (1H, m), 1.60 (1H, s), 1.30-1.20 (1H, m), 1.25 (3H, s). ¹³C NMR (CDCl₃, 100 MHz) δ: 170.33, 164.18, 135.42, 134.90, 132.83, 132.37, 129.45, 127.63, 127.12, 126.84, 125.69, 119.98, 76.84, 55.65, 55.03, 51.04, 48.97, 45.39, 39.16, 29.84, 28.87, 28.17, 26.21, 24.46. IR νₘₐₓ (film)/cm⁻¹ 2924, 2854, 2096, 1649, 1259, 1114. HRMS (ESI) calcd for C₂₇H₂₈BF₂N₇O₂ [M+Na]⁺553.2294 m/z, found 553.2283 m/z.

### 5,5-Difluoro-7-(3-oxo-3-(4-oxo-3,4,6,7-tetrahydro-1H-pyrazino[2,1-a]isoquinolin-2(11bH)-yl)propyl)-3-(3-(4-(2-((E)-3-((R)-2,2,5,5-tetramethyl-1,3-dioxane-4-carboxamido)acrylamido)ethyl)-1H-1,2,3-triazol-1-yl)propyl)-5H-dipyrrolo[1,—2-c:2′,1′-f][1,3,2]diazaborinin-4-ium-5-uide, 13

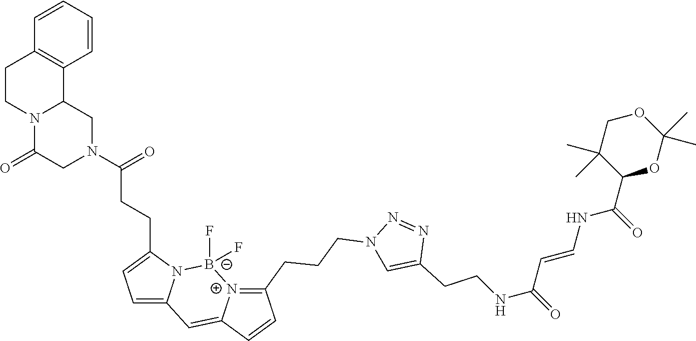 [SMILES: CC1(C)OCC(C)(C)[C@H](C(=O)N/C=C/C(=O)NCCc2cn(CCCC3=[N+]4C(=Cc5ccc(CCC(=O)N6CC(=O)N7CCc8ccccc8C7C6)n5[B-]4(F)F)C=C3)nn2)O1]

A solution of PZQ-BODIPY-azide (37 mg, 70 μmol) and of pantothenate derived alkyne (22 mg, 70 μmol) in dry THF (1.5 mL ) was treated with catalytic amounts of CuI and DIPEA. The resulting mixture was heated at 40° C. and stirred overnight. Once the TLC showed the consumption of starting material (CH₂Cl₂:MeOH; 96:4), the reaction was allowed to cool down to r.t. and the crude product was purified by column chromatography. The compound was obtained as a dark red solid in 20% yield (11 mg) as a mixture of triazoles.

¹H NMR (CDCl₃, 400 MHz) δ 8.30 (1H, d, J=11.3 Hz), 7.81 (1H, dt, J=20.3, 10.2 Hz), 7.44 (1H, s), 7.35 (1H, s), 7.26-6.92 (8H, m), 6.46-6.28 (2H, m), 5.75 (1H, dd, J=13.8, 4.5 Hz), 5.15 (1H, dd, J=13.3, 3.0 Hz), 4.88-4.68 (2H, m), 4.47-4.32 (3H, m), 4.15 (1H, dd, J=6.8, 5.2 Hz), 4.04 (1H, d, J=17.5 Hz), 3.91-3.57 (4H, m), 3.39-3.24 (3H, m), 3.07-2.71 (8H, m), 2.37 (2H, dq, J=15.0, 7.5 Hz), 2.17 (2H, s), 1.46 (3H, s), 1.38 (3H, s) 0.97 (3H, s), 0.87 (3H, s). ¹³C NMR (CDCl₃, 100 MHz) δ 168.1, 164.2, 134.9, 129.4, 127.1, 99.5, 76.8, 71.4, 50.9, 33.4, 29.5, 22.0, 18.9, 18.8. ¹⁹F NMR (377 MHz, CDCl₃) δ −141.87, −141.95, −142.05, -142.14, −142.35, −142.44, −142.53, −142.62. HRMS (ESI) calcd for C₄₃H₅₂BF₂N₉O₆ [M+Na]⁺ 861.4030 m/z, found 861.4004 m/z.

### Compound Syntheses—Coumarin Conjugate

### Ethyl 2-(7-(dimethylamino)-2-oxo-2H-chromen-4-yl)acetate

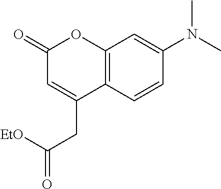 [SMILES: CCOC(=O)Cc1cc(=O)oc2cc(N(C)C)ccc12]

ZnCl₂ was stirred at 140° C. under vacuum for 18 h before finally being flame-dried and then cooled down prior to weighing. A solution of 3-(dimethylamino)phenol (2.92 mL , 16.0 mmol) in EtOH (10 mL ) was heated with ethyl acetonedicarboxylate (2.00 g, 14.6 mmol) followed by ZnCl₂ (2.38 g, 17.5 mmol). The resultant suspension was then heated at reflux for 16 h before being cooled down to room temperature and poured over ice (50 g). The aqueous phase was extracted with CH₂Cl₂ (3×30 mL ) before the combined organics were washed with brine (50 mL ), dried (Na₂SO₄), filtered and concentrated in vacuo to afford the crude product as a dark purple oil. Purification of the crude residue by flash column chromatography (20-50% EtOAc/petroleum ether) afforded the coumarin product as a pale yellow solid (1.94 g, 48%). The NMR data is in accordance with the literature (see Ma et al.).

¹H NMR (CDCl₃, 400 MHz) δ: 7.40 (1H, d, J=9.0 Hz), 6.61 (1H, dd, J=9.0, 2.6 Hz), 6.51 (1H, d, J=2.6 Hz), 6.05 (1H, brs), 4.18 (2H, q, J=7.1 Hz), 3.67 (2H, d, J=0.9 Hz), 3.05 (6H, s), 1.25 (3H, t, J=7.1 Hz); ¹³C NMR (CDCl₃, 100 MHz) δ: 169.1, 161.7, 156.0, 153.0, 148.4, 125.3, 110.7, 109.0, 108.5, 98.4, 61.5, 40.1, 38.2, 14.1.

### 2-(7-(Dimethylamino)-2-oxo-2H-chromen-4-yl)acetic Acid

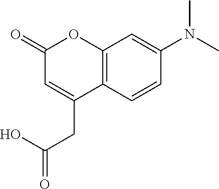 [SMILES: CN(C)c1ccc2c(CC(=O)O)cc(=O)oc2c1]

To a solution of ethyl 2-(7-(dimethylamino)-2-oxo-2H-chromen-4-yl)acetate (1.92 g, 6.97 mmol) in THF (42 mL ) was added a solution of LiOH (338 mg, 14.1 mmol) in H₂O (85 mL ). The resultant solution was stirred for 4 h at room temperature at which time the reaction mixture was washed with Et2O (2×100 mL ). The aqueous layer was then acidified to pH 2 with 1 M HCl, and the resultant yellow precipitate was filtered off to yield the product acid (1.24 g, 72%) as a yellow solid. The NMR data is in accordance with the literature (see Ma et al.).

¹H NMR ((CD₃)₂CO, 400 MHz) δ: 7.56 (1H, d, J=8.9 Hz), 6.76 (1H, dd, J=8.9, 2.6 Hz), 6.54 (1H, d, J=2.6 Hz), 6.08 (1H, brs), 3.85 (2H, d, J=0.7 Hz), 3.10 (6H, s), 2.88 (1H, br s); ¹³C NMR ((CD₃)₂CO, 100 MHZ) δ: 170.8, 161.4, 157.0, 154.1, 150.3, 126.7, 111.2, 109.8, 109.4, 98.7, 40.2, 37.9.

### N-(3-Azidopropyl)-2-(7-(dimethylamino)-2-oxo-2H-chromen-4-yl)acetamide

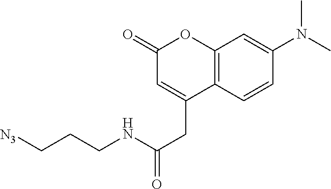 [SMILES: CN(C)c1ccc2c(CC(=O)NCCCN=[N+]=[N-])cc(=O)oc2c1]

A solution of 3-azidopropan-1-amine (50 mg, 0.500 mmol) (known from Zabrodski et al.) in CH₂Cl₂ (1.5 mL ) was cooled down to 0° C. before 2-(7-(dimethylamino)-2-oxo-2H-chromen-4-yl)acetic acid (49 mg, 0.200 mmol), EDC (46 mg, 0.240 mmol), DIPEA (70 μL, 0.400 mmol) and a catalytic amount of DMAP were added. After 15 min stirring at 0° C., the reaction mixture became cloudy with the formation of a white precipitate. A further portion of CH₂Cl₂ (3 mL ) was added and the mixture sonicated to aid stirring. The slurry was allowed to warm to room temperature while stirring for 16 h after which the suspension had became a yellow solution. The reaction mixture was concentrated in vacuo to give the crude product as a yellow oil. Purification of the crude residue by flash column chromatography (0-2% MeOH/CH₂Cl₂) afforded the azide product (40 mg, 84%) as a yellow solid.

¹H NMR ((CD₃)₂CO, 400 MHz) δ: 7.58 (1H, d, J=9.0 Hz), 7.45 (1H, brs), 6.71 (1H, dd, J=9.0, 2.6 Hz), 6.49 (1H, d, J=2.6 Hz), 6.02 (1H, br s), 3.66 (2H, d, J=0.7 Hz), 3.34 (2H, t, J=6.9 Hz), 3.30-3.25 (2H, m), 3.07 (6H, s), 1.77-1.70 (2H, m); ¹³C NMR ((CD₃)₂CO, 100 MHz) δ: 168.7, 161.5, 156.9, 154.1, 151.3, 126.9, 111.1, 109.7, 109.5, 98.6, 49.7, 40.5, 40.2, 37.5, 29.6; IR νmax (film)/cm⁻¹ 3283, 2920, 2881,2807, 2085, 1705, 1604; HRMS (EI) calcd for C₁₆H₁₈O₃N₅ (M−H)+: m/z 328.1415, found m/z 328.1404.

### (R,E)-N-(3-(2-(1-(3-(2-(7-(Dimethylamino)-2-oxo-2H-chromen-4-yl)acetamido)propyl)-1H-1,2,3-triazol-4-yl)ethylamino)-3-oxoprop-1-enyl)-2,2,5,5-tetramethyl-1,3-dioxane-4-carboxamide

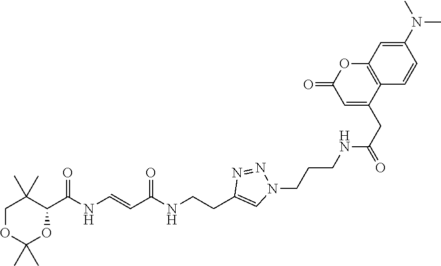

(R,E)-N-(3-(But-3-ynylamino)-3-oxoprop-1-enyl)-2,2,5,5-tetramethyl-1,3-dioxane-4-carboxamide (59 mg, 0.192 mmol) and N-(3-azidopropyl)-2-(7-(dimethylamino)-2-oxo-2H-chromen-4-yl)acetamide (46 mg, 0.192 mmol) were dissolved in THF (2 mL ). One drop of DIPEA was added via pipette, followed by a catalytic amount of CuI. The resultant mixture was heated to 40° C. for 18 h, before reducing in vacuo to remove solvent. The crude residue was purified by flash column chromatography (0-5% MeOH/CH₂Cl₂) to afford the desired compound (78 mg, 74%) as a yellow oil.

¹H NMR ((CD₃)₂CO, 500 MHz) δ: 9.42 (1H, d, J=11.0 Hz), 7.81 (1H, dd, J=14.0, 11.0, Hz), 7.69 (1H, s), 7.60 (1H, d, J=9.0 Hz), 7.59 (1H, t, J=5.7 Hz), 7.21 (1H, t, J=5.7 Hz), 6.72 (1H, dd, J=9.0, 2.6 Hz), 6.48 (1H, d, J=2.6 Hz), 6.02 (1H, br s), 5.95 (1H, d, J=14.0 Hz), 4.35 (2H, t, J=6.9 Hz), 4.26 (1H, s), 3.75 (1H, d, J=11.6 Hz), 3.66 (2H, s), 3.53-3.50 (2H, m), 3.26 (1H, d, J=11.6 Hz), 3.22-3.19 (2H, m), 3.06 (6H, s), 2.39 (2H, t, J=6.9 Hz), 2.09-2.02 (2H, m), 1.44 (3H, s), 1.37 (3H, s), 1.00 (3H, s), 0.98 (3H, s); ¹³C NMR ((CD₃)₂CO, 125 MHz) δ: 169.3, 168.7, 167.0, 161.6, 156.9, 154.1, 151.4, 145.8, 133.8, 126.9, 123.0, 111.1, 109.8, 109.5, 106.9, 100.0, 98.6, 77.9, 71.7, 48.1, 40.4, 40.2, 39.6, 37.3, 33.8, 30.9, 29.7, 26.8, 22.1, 19.3, 19.0; HRMS (ESI) calcd for C₃₂H₄₂N₇O₇ (M−H)+: m/z 636.3151, found m/z 636.3133; m.p. 141-142° C.

### (R,E)-N-(3-(2-(1-(3-(2-(7-(Dimethylamino)-2-oxo-2H-chromen-4-yl)acetamido)propyl)-1H-7,2,3-triazol-4-yl)ethylamino)-3-oxoprop-1-enyl)-2,4-dihydroxy-3,3-dimethylbutan-amide

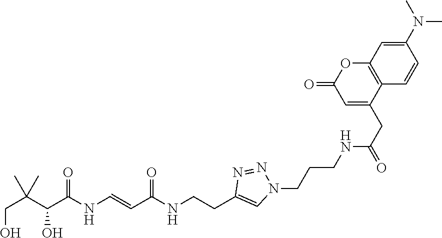 [SMILES: CN(C)c1ccc2c(CC(=O)NCCCn3cc(CCNC(=O)/C=C/NC(=O)[C@H](O)C(C)(C)CO)nn3)cc(=O)oc2c1]

(R,E)-N-(3-(But-3-ynylamino)-3-oxoprop-1-enyl)-2,4-dihydroxy-3,3-dimethylbutanamide (49 mg, 0.183 mmol) and N-(3-azidopropyl)-2-(7-(dimethylamino)-2-oxo-2H-chromen-4-yl)acetamide (44 mg, 0.183 mmol) were dissolved in THF (2 mL ). One drop of DIPEA was added via pipette, followed by a catalytic amount of CuI. The resultant mixture was heated to 70° C. for 18 h, before concentrating in vacuo to remove solvent to afford the crude product as a yellow oil. Purification of the crude residue by flash column chromatography (0-10% 7 M NH₃ in MeOH/CH₂Cl₂) gave the desired compound (33 mg, 30%) as a yellow oil.

¹H NMR ((CDS)₂SO, 500 MHz) δ: 10.20 (1H, d, J=10.9 Hz), 8.31 (1H, t, J=5.5 Hz), 7.95 (1H, t, J=5.7 Hz), 7.87 (1H, s), 7.62 (1H, dd, J=11.0, 10.9 Hz), 7.55 (1H, d, J=9.0 Hz), 6.73 (1H, dd, J=9.0, 2.6 Hz), 6.55 (1H, d, J=2.6 Hz), 6.01 (1H, brs), 5.86 (1H, d, J=14.0 Hz), 5.68 (1H, br s), 5.51 (1H, br s), 4.31 (2H, t, J=6.9 Hz), 3.87 (1H, s), 3.62 (2H, s), 3.38-3.32 (3H, m), 3.16 (1H, d, J=10.4 Hz), 3.09-3.05 (2H, m), 3.02 (6H, s), 2.76 (2H, t, J=7.3 Hz), 1.96-1.91 (2H, m), 0.83 (3H, s), 0.80 (3H, s); ¹³C NMR ((CDs)₂SO, 125 MHz) δ: 170.5, 168.0, 166.1, 160.7, 155.4, 152.8, 151.2, 145.4, 133.0, 125.9, 122.2, 109.4, 109.0, 108.2, 105.1, 97.4, 74.9, 67.4, 46.9, 39.7, 39.6, 39.2, 38.8, 38.5, 29.7, 25.7, 21.1, 19.9; IR νmax (film)/cm⁻¹ 3295, 2945, 2929, 2876, 1711, 1653, 1615, 1598; HRMS (ESI) calcd for C₂₉H₃₈N₇O₇ (M−H)₊: m/z 596.2838, found m/z 596.2822; m.p. 146-147° C.

### Compound Syntheses—Fenbendazole Conjugate

### 5-(Phenylthio)-1H-benzo[d]imidazol-2-amine

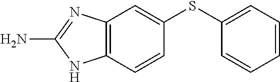 [SMILES: Nc1nc2cc(Sc3ccccc3)ccc2[nH]1]

To a suspension of fenbendazole (1.00 g, 3.34 mmol) in DMSO (12 mL ) and water (4 mL ) was added potassium hydroxide (750 mg, 13.4 mmol). The resultant mixture was heated to 80° C., causing the material to go in to solution. After heating for 72 h, the mixture was allowed to cool to room temperature before diluting with EtOAc (100 mL ) and water (100 mL ). The layers were separated and the aqueous washed with further portions of EtOAc (2×75 mL) before combined organic layers washed with brine (100 mL ), dried over sodium sulphate and concentrated in vacuo to give the desired guanidine (796 mg, 99%) as a light purple solid.

¹H NMR ((CD₃)₂CO, 400 MHz) δ: 10.17 (1H, brs), 7.35 (1H, dd, J=1.7, 0.5 Hz), 7.27-7.22 (3H, m), 7.14-7.10 (4H, m), 5.97 (2H, br s).

### tert-Butyl 3-oxo-3-(5-(phenylthio)-1H-benzo[d]imidazol-2-ylamino)propylcarbamate

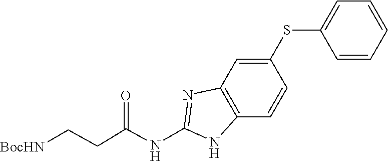 [SMILES: CC(C)(C)OC(=O)NCCC(=O)Nc1nc2cc(Sc3ccccc3)ccc2[nH]1]

5-(Phenylthio)-1H-benzo[d]imidazol-2-amine (116 mg, 0.482 mmol) and N-(tert-butoxycarbonyl)-L-alanine (137 mg, 0.723 mmol) were dissolved in DMF (4.5 mL) to give a purple solution. The mixture was cooled to 0° C. before HBTU (292 mg, 0.771 mmol) and DIPEA (134 μL, 0.771 mmol) were added and allowed to stir for 16 h. The reaction mixture was diluted with EtOAc (30 mL) and subsequently washed with 1 M NaOH (25 mL), water (25 mL) and brine (4×20 mL) before drying (Na₂SO₄) and reducing in vacuo to afford the crude product as a yellow oil. Purification by silica gel column chromatography (0-2% MeOH/CH₂Cl₂) gave the desired product (185 mg, 93%) as a white solid.

¹H NMR ((CD₃)₂CO, 400 MHz) δ: 11.87 (2H, brs), 7.80 (1H, s), 7.70 (1H, d, J=8.1 Hz), 7.40-7.36 (3H, m), 7.31-7.26 (3H, m), 6.25 (1H, br appt), 3.60-3.56 (2H, m), 2.93 (2H, t, J=6.7 Hz), 1.47 (9H, s). HRMS (ESI) calcd for C₂₁H₂₄N₄NaO₃S (M+Na)⁺: m/z 435.1461, found m/z 435.1443.

### (R,E)-2,2,5,5-Tetramethyl-N-(3-oxo-3-(3-oxo-3-(5-(phenylthio)-1H-benzo[d]imidazol-2-ylamino)propylamino)prop-1-enyl)-1,3-dioxane-4-carboxamide

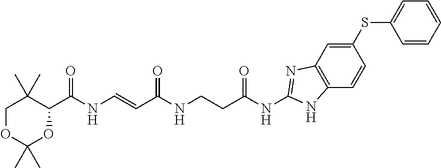 [SMILES: CC1(C)OCC(C)(C)[C@H](C(=O)N/C=C/C(=O)NCCC(=O)Nc2nc3cc(Sc4ccccc4)ccc3[nH]2)O1]

tert-Butyl 3-oxo-3-(5-(phenylthio)-1H-benzo[d]imidazol-2-ylamino)propylcarbamate (60 mg, 0.145 mmol) was dissolved in a mixture of CH₂Cl₂ (1 mL) and TFA (1 mL ) and allowed to stir at room temperature for 2 h. The reaction mixture was then reduced in vacuo to afford the crude TFA salt as a pink oil. The crude salt was dissolved in DMF (1.5 mL) and cooled to 0° C. before sequential addition of (R,E)-3-(2,2,5,5-tetramethyl-1,3-dioxane-4-carboxamido)acrylic acid (27 mg, 0.106 mmol), HBTU (60 mg, 0.159 mmol) and DIPEA (56 μL, 0.318 mmol). The resultant mixture was allowed to stir for 16 h at room temperature before diluting with EtOAc (15 mL) and washing with saturated NaHCO₃ (15 mL), water (15 mL) and brine (4×10 mL) before drying (Na₂SO₄) and reducing in vacuo to afford the crude product as a yellow oil. Purification by silica gel column chromatography (0-5% MeOH/CH₂Cl₂) gave the desired product (31 mg, 53%) as a cream coloured solid.

¹H NMR ((CD₃)₂CO, 500 MHz) δ: 11.33 (1H, brs), 11.30 (1H, brs), 9.41 (1H, d, J=10.8 Hz), 7.85 (1H, dd, J=14.0, 10.8 Hz), 7.69 (1H, s), 7.58 (1H, d, J=8.2 Hz), 7.33-7.18 (7H, m), 5.96 (1H, d, J=14.0 Hz), 4.23 (1H, s), 3.78 (1H, d, J=11.7 Hz), 3.68-3.65 (2H, m), 3.29 (1H, d, J=11.7 Hz), 2.86 (2H, t, J=6.5 Hz), 1.47 (3H, s), 1.39 (3H, s), 1.03 (3H, s), 1.00 (3H, s).

### Compound Syntheses—Albendazole Conjugates

### 5-(Propyl)-1H-benzo[d]imidazol-2-amine

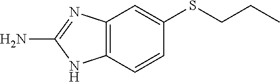

To a suspension of albendazole (886 mg, 3.34 mmol) in MeOH (25 mL) and water (6.5 mL) was added potassium hydroxide (375 mg, 6.68 mmol). The resultant mixture was heated at reflux, giving a yellow solution. After heating for 72 h, TLC analysis showed starting material present. A further portion of potassium hydroxide (375 mg, 6.68 mmol) was added and the mixture stirred for a further 24 h. The mixture was allowed to cool to room temperature before removal of MeOH in vacuo. The remaining aqueous was then diluted with CH₂Cl₂ (3×20 mL ) before combined organic layers washed with brine (50 mL ), dried over sodium sulfate and concentrated in vacuo to give the desired guanidine (501 mg, 72%) as a light grey solid.

¹H NMR ((CD₃)₂SO, 400 MHz) δ: 10.67 (1H, brs), 7.14 (1H, d, J=1.5 Hz), 7.03 (1H, dd, J=8.1, 0.5 Hz), 6.92 (1H, br), 6.20 (2H, s), 2.79 (2H, t, J=7.3 Hz), 1.56-1.47 (2H, m), 0.94 (3H, t, J=7.3 Hz). Reference literature: Zhao et al. Org. Biomol. Chem. 2010, 8, 3328-3337.

### (R,E)-2,2,5,5-Tetramethyl-N-[3-oxo-3-[(5-propylsulfanyl-1H-benzimidazol-2-yl) amino]prop-1-enyl]-1,3-dioxane-4-carboxamide

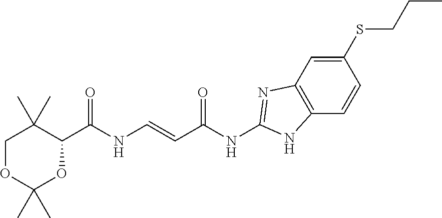 [SMILES: CCCSc1ccc2[nH]c(NC(=O)/C=C/NC(=O)[C@@H]3OC(C)(C)OCC3(C)C)nc2c1]

5-(Propyl)-1H-Benzo[d]imidazol-2-amine (33 mg, 0.159 mmol) and (R,E)-3-(2,2,5,5-tetramethyl-1,3-dioxane-4-carboxamido)acrylic acid (27 mg, 0.106 mmol) were dissolved in DMF (1 mL ) at 0° C. HBTU (60 mg, 0.159 mmol) and DIPEA (28 μL, 0.159 mmol) were added and the resultant mixture allowed to stir for 16 h while warming to rt. The reaction was diluted with EtOAc (15 mL ) and washing with saturated NaHCO₃ (15 mL ), water (15 mL ) and brine (4×10 mL ) before drying (Na₂SO₄) and reducing in vacuo to afford the crude product as a yellow oil. Multiple purification attempts by silica gel column chromatography (0-5% MeOH/EtOAc) gave the desired product (9 mg, 17%) as a white solid.

¹H NMR (CDCl₃, 500 MHz) δ: 11.65 (1H, br), 7.82 (1H, dd, J=12.0, 8.9 Hz), 7.40 (1H, d, J=1.7 Hz), 7.37 (1H, d, J=8.4 Hz), 7.11 (1H, dd, J=8.4, 1.7 Hz), 6.33 (2H, br), 6.08 (1H, d, J=12.0 Hz), 4.31 (1H, s), 3.78 (1H, d, J=11.8 Hz), 3.39 (1H, d, J=11.8 Hz), 2.92 (2H, t, J=7.3 Hz), 1.72-1.65 (2H, m), 1.64 (3H, s), 1.52 (3H, s), 1.10 (6H, s), 1.03 (3H, t, J=7.3 Hz). ¹³C NMR (CDCl₃, 125 MHz) δ: 169.2, 167.9, 154.5, 140.7, 132.7, 124.3, 122.8, 118.4, 112.8, 99.4, 97.2, 77.4, 71.2, 36.8, 33.4, 29.7, 29.3, 22.6, 21.8, 19.1, 18.7, 13.4. HRMS (ESI) calcd for C₂₂H₃₁N₄O₄S (M+H)⁺: m/z 447.2066, found m/z 447.2037.

### tert-Butyl 3-oxo-3-(5-(propyl)-1H-benzo[d]imidazol-2-ylamino)propylcarbamate

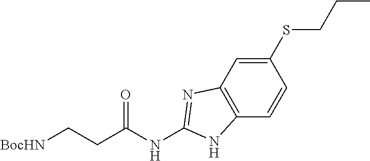 [SMILES: CCCSc1ccc2[nH]c(NC(=O)CCNC(=O)OC(C)(C)C)nc2c1]

5-(Propyl)-1H-benzo[d]imidazol-2-amine (100 mg, 0.482 mmol) and N-(tert-butoxycarbonyl)-L-alanine (137 mg, 0.723 mmol) were dissolved in DMF (4.5 mL ) to give a purple solution. The mixture was cooled to 0° C. before HBTU (292 mg, 0.771 mmol) and DIPEA (134 μL, 0.771 mmol) were added and allowed to stir for 16 h. The reaction mixture was diluted with EtOAc (30 mL ) and subsequently washed with 1M NaOH (25 mL ), water (25 mL ) and brine (4×20 mL ) before drying (Na₂SO₄) and reducing in vacuo to afford the crude product as a yellow oil. Purification by silica gel column chromatography (0-2% MeOH/CH₂Cl₂) gave the desired product (125 mg, 69%) as a white solid.

¹H NMR ((CD₃)₂SO, 500 MHz) δ: 12.06 (1H, br), 11.54 (1H, s), 7.50-7.36 (2H, m), 7.11 (1H, d, J=7.9 Hz), 6.91 (1H, t, J=5.6 Hz), 3.29-3.25 (2H, m), 2.86 (2H, t, J=7.2 Hz), 2.59 (2H, t, J=7.0), 1.57-1.50 (2H, m), 1.37 (9H, s), 0.95 (3H, t, J=7.2 Hz). HRMS (ESI) calcd for C₁₈H₂₆N₄NaO₃S (M+Na)⁺: m/z 401.1618, found m/z 401.1596.

### (R,E)-2,2,5,5-Tetramethyl-N-(3-oxo-3-(3-oxo-3-(5-(propyl)-1H-benzo[d]imidazol-2-ylamino)propylamino) prop-1-enyl)-1,3-dioxane-4-carboxamide

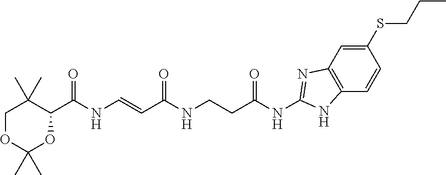 [SMILES: CCCSc1ccc2[nH]c(NC(=O)CCNC(=O)/C=C/NC(=O)[C@@H]3OC(C)(C)OCC3(C)C)nc2c1]

tert-Butyl 3-oxo-3-(5-(propyl)-1H-benzo[d]imidazol-2-ylamino)propylcarbamate (60 mg, 0.159 mmol) was dissolved in a mixture of CH₂Cl₂ (1 mL ) and TFA (1 mL ) and allowed to stir at room temperature for 2 h. The reaction mixture was then reduced in vacuo to afford the crude TFA salt as a pink oil. The crude salt was dissolved in DMF (1.5 mL ) and cooled to 0° C. before sequential addition of (R,E)-3-(2,2,5,5-tetramethyl-1,3-dioxane-4-carboxamido)acrylic acid (27 mg, 0.106 mmol), HBTU (60 mg, 0.159 mmol) and DIPEA (56 μL, 0.318 mmol). The resultant mixture was allowed to stir for 16 h at room temperature before diluting with EtOAc (15 mL ) and washing with saturated NaHCO₃ (15 mL ), water (15 mL ) and brine (4×10 mL ) before drying (Na₂SO₄) and reducing in vacuo to afford the crude product as a off-white solid. Purification by silica gel column chromatography (0-10% MeOH/EtOAc) gave the desired product (32 mg, 58%) as a white solid.

¹H NMR ((CD₃)₂CO, 500 MHz) δ: 11.52 (2H, br), 9.38 (1H, d, J=10.8 Hz), 7.83 (1H, dd, J=14.0, 10.8 Hz), 7.59 (1H, s), 7.45 (1H, d, J=8.4 Hz), 7.28 (1H, t, J=5.7 Hz), 7.20 (1H, dd, J=8.4, 1.7 Hz), 5.93 (1H, d, J=14.0 Hz), 4.26 (1H, s), 3.75 (1H, d, J=11.8 Hz), 3.66-3.62 (2H, m), 3.26 (1H, d, J=11.8 Hz), 2.88 (2H, t, J=7.3 Hz), 2.83 (2H, t, J=6.5 Hz), 1.64-1.56 (2H, m), 1.44 (3H, s), 1.36 (3H, s), 1.00-0.97 (9H, m); HRMS (ESI) calc for C₂₅H₃₆N₅O₅S (M+H)⁺518.2437; found 518.2406.

### Compound Syntheses—Protein Conjugates

### tert-Butyl (5-hydroxypenthyl)carbamate

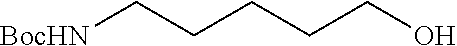 [SMILES: CC(C)(C)OC(=O)NCCCCCO]

To a 100 mL flask were added 5-aminopentanol (500 mg, 4.84 mmol) and CH₂Cl₂ (10 mL ) and the mixture stirred for five min, before the addition of Et₃N (1.3 mL , 9.6 mmol) followed by the dropwise addition of a solution of BoC₂O (1.16 g, 5.3 mmol) in CH₂Cl₂ (10 mL ). The reaction mixture was then stirred at r.t. for 16 h, after which it was diluted with water (20 mL ). The phases were separated, and the aqueous phase was extracted with CH₂Cl₂ (2×20 mL ). The combined organics were washed with brine (60 mL ), dried over Na₂SO₄ and filtered. Solvent was removed in vacuo to yield a yellow oil. The crude product was purified by chromatography (CH₃OH/CH₂Cl₂ (1-5%)), to yield the desired alcohol as a yellow oil (730 mg, 74%).

¹H NMR (CDCl₃, 400 MHz) δ: 4.5 (1H, brs), 3.67 (2H, t, J=6.4 Hz), 3.15 (2H, dd, J=12.8, 6.3 Hz), 1.6-1.49 (4H, m), 1.45 (9H, s), 1.44-1.39 (2H, m). ¹³C NMR (CDCl₃, 100 MHz) δ: 156.0, 79.1, 62.7, 40.4, 32.2, 29.8, 28.4, 22.9.

### tert-Butyl (5-(2,5-dioxo-2,5-dihydro-1H-pyrrol-1-yl)pentyl)carbamate

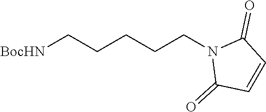 [SMILES: CC(C)(C)OC(=O)NCCCCCN1C(=O)C=CC1=O]

To a 25 mL flask were added tert-butyl (5-hydroxypenthyl)carbamate (203 mg, 1 mmol), maleimide (107 mg, 1.1 mmol), triphenylphosphine (283 mg, 1.08 mmol) and THF (5 mL ). The mixture was stirred for 10 min at room temperature before the dropwise addition of DIAD (0.23 mL , 1.2 mmol). The reaction mixture was stirred for a further 16 h at r.t. at which point, the solvent was reduced in vacuo to yield a crude yellow oil. Purification of the crude residue by flash chromatography (CH₃OH/CH₂Cl₂ (0-1%)) yielded the desired compound (248 mg, 87%).

¹H NMR (CDCl₃, 400 MHz) δ: 6.70 (2H, s), 4.5 (1H, brs), 3.53 (2H, t, J=7.2 Hz), 3.12 (2H, dd, J=12.6, 6.2 Hz), 1.67-1.57 (2H, m), 1.56-1.47 (2H, m), 1.46 (9H, s), 1.36-1.30 (2H, m). ¹³C NMR (CDCl₃, 100 MHz) δ: 170.8, 156.3, 134.0, 79.1, 70.1, 40.3, 37.6, 29.8, 28.4, 23.9.

### tert-Butyl (3-(2,5-dioxo-2,5-dihydro-1H-pyrrol-1-yl)propyl)carbamate

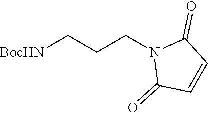 [SMILES: CC(C)(C)OC(=O)NCCCN1C(=O)C=CC1=O]

To a 25 mL flask were added tert-butyl (3-hydroxypropyl)carbamate (0.25 mL, 1.5 mmol), maleimide (175 mg, 1.8 mmol), PPh₃ (472 mg, 1.8 mmol) and THF (7 mL). The mixture was stirred for 10 min at r.t. before the dropwise addition of DIAD (0.35 mL, 1.8 mmol). The reaction was then stirred for a further 18 h at r.t. and the solvent was reduced in vacuo to yield a crude yellow oil. This crude oil was purified by column chromatography (ethyl acetate/petroleum ether (0-30%)) to yield a colourless oil (257 mg, 69%).

¹H NMR (CDCl₃, 400 MHz) δ: 6.63 (2H, s), 5.08 (1H, brs), 3.55-3.42 (2H, t, J=6.7 Hz), 3.06-2.9 (2H, dd, J=11.9, 5.8 Hz), 1.73-1.63 (2H, q, J=6.7 Hz),1.38 (9H, s). ¹³C NMR (CDCl₃, 100 MHz) δ: 170.9, 170.3, 135.1, 79.3, 70.1, 37.3, 34.9, 28.4.

### 1-(5-Aminopentyl)-1H-pyrrole-2,5-dione 2,2,2-trifluoroacetate Salt

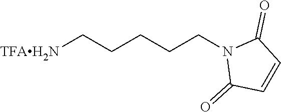

To a 10 mL flask were added tert-butyl (5-(2,5-dioxo-2,5-dihydro-1H-pyrrol-1-yl)pentyl)carbamate (140 mg, 0.49 mmol), CH₂Cl₂ (0.6 mL) and trifluoroacetic acid (0.6 mL). The resulting mixture was then stirred at r.t. for 2.5 h, after which, the reaction mixture was diluted with CH₂Cl₂ (2 mL) and quenched with water (5 mL). The phases were separated and the organic phase was washed with water (5×3 mL). The combined aqueous phases were then extracted with CH₂Cl₂ (3×5 mL) before water was removed under high vacuum over 36 h. The product was obtained as white crystals (107 mg, 73%).

¹H NMR (MeOD, 400 MHz) δ: 6.71 (2H, s), 3.40 (2H, t, J=7.2 Hz), 2.80 (2H, t, J=7.2 Hz), 1.62-1.49 (4H, m), 1.30-1.23 (2H, m). ¹³C NMR (MeOD, 125 MHz) δ: 171.2, 133.9, 39.1, 36.6, 27.6, 26.5, 23.1. ¹⁹F NMR (MeOD, 470 MHz) δ: −76.8.

### 3-(2,5-Dioxo-2,5-dihydro-1H-pyrrol-1-yl)propan-1-aminium 2,2,2-trifluoroacetate Salt

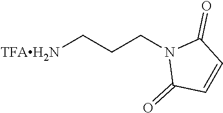

To a 10 mL flask were added tert-butyl (3-(2,5-dioxo-2,5-dihydro-1H-pyrrol-1-yl)propyl)carbamate (250 mg, 0.98 mmol), CH₂Cl₂ (1.5 mL) and trifluoroacetic acid (1.5 mL). The mixture was stirred at room temperature for 3 h. After this time the reaction mixture was diluted with CH₂Cl₂ (2 mL) before addition of water (5 mL). The organic phase was washed with water (5×3 mL). The combined aqueous phases were then washed with CH₂Cl₂ (3×5 mL) before water was removed in vacuo at high vacuum over 36 h. The product was obtained as white solid (250 mg, 95%).

¹H NMR (MeOD, 500 MHz) δ: 6.86 (2H, s), 3.63 (2H, t, J=7.0 Hz), 2.95 (2H, t, J=7.5 Hz), 1.97-1.91 (2H, m). ¹³C NMR (MeOD, 125 MHz) δ: 171.1, 134.1, 37.0, 34.0, 26.5. ¹⁹F NMR (MeOD, 470 MHz) δ: −76.8 (3F).

### Perfluorophenyl (R,E)-3-(2,2,5,5-tetramethyl-1,3-dioxane-4-carboxamido)acrylate

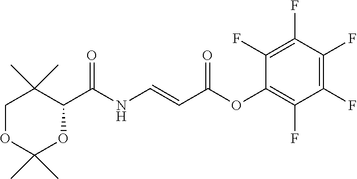 [SMILES: CC1(C)OCC(C)(C)[C@H](C(=O)N/C=C/C(=O)Oc2c(F)c(F)c(F)c(F)c2F)O1]

To a 10 mL flask were added (R,E)-3-(2,2,5,5-tetramethyl-1,3-dioxane-4-carboxamido)acrylic acid (100 mg, 0.38 mmol), 2,3,4,5,6-pentafluorophenol (90 mg, 0.48 mmol), N-(3-dimethylaminopropyl)-N′-ethylcarbodiimide hydrochloride (88 mg, 0.45 mmol), 4-(dimethylamino)pyridine (2 mg, 0.016 mmol) and CH₂Cl₂ (3 mL). The resulting mixture was then stirred at r.t. for 24 h. At this point, the solvent was reduced in vacuo and the crude was purified by flash column chromatography (CH₂Cl₂ (100%)). After purification the starting material acrylic acid was recovered (10 mg, 10%), together with the expected product as a yellow oil (60 mg, 40%) and the corresponding Z-isomer also as yellow oil (60 mg, 40%).

¹H NMR (CDCl₃, 500 MHz) δ: 8.61 (1H, d, J=12.5 Hz), 8.1 (1H, dd, J=12.5, 12.5 Hz), 5.76 (1H, d, J=14 Hz), 4.18 (1H, s), 3.66 (1H, d, J=12.0 Hz), 3.27 (1H, d, J=12.0 Hz), 1.45 (3H, s), 1.40 (3H, s), 0.99 (3H, s), 0.96 (3H, s). ¹³C NMR (CDCl₃, 125 MHz) δ: 168.2, 162.9, 140.2, 140.0, 138.8, 136.8, 99.7, 98.8, 77.0, 71.2, 33.5, 29.4, 21.7, 18.9, 18.8. ¹⁹F NMR (CDCl₃, 470 MHz) δ: −152.0, −158.0, −163.0. HRMS (ESI) calcd for C₁₈H₁₈F₅NO₅ M+: m/z 423.1105, found m/z 423.1081.

### Perfluorophenyl (R,Z)-3-(2,2,5,5-tetramethyl-1,3-dioxane-4-carboxamido)acrylate

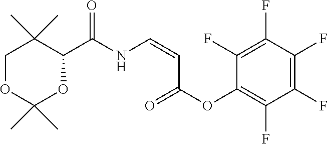 [SMILES: CC1(C)OCC(C)(C)[C@H](C(=O)N/C=C\C(=O)Oc2c(F)c(F)c(F)c(F)c2F)O1]

To a 10 mL flask were added (R,Z)-3-(2,2,5,5-tetramethyl-1,3-dioxane-4-carboxamido)acrylic acid (35 mg, 0.13 mmol), 2,3,4,5,6-pentafluorophenol (38 mg, 0.20 mmol), N-(3-dimethylaminopropyl)-N′-ethylcarbodiimide hydrochloride (38 mg, 0.19 mmol), 4-(dimethylamino)pyridine (3 mg, 0.02 mmol) and CH₂Cl₂ (2 mL). The reaction mixture was stirred at r.t. for 18 h and then the solvent was reduced in vacuo. The crude residue was then purified by flash column chromatography (petroleum ether:ethyl acetate (0-20%)). After purification, the desired ester was obtained as a yellow oil (40 mg, 64%) together with the corresponding E-isomer as yellow oil (22 mg, 35%).

¹H NMR (CDCl₃, 500 MHz) δ: 10.80 (1H, d, J=11.5 Hz), 7.63-7.59 (1H, dd, J=9.0, 8.5 Hz), 5.39 (1H, d, J=8.5 Hz), 4.16 (1H, s), 3.64 (1H, d, J=12.0 Hz), 3.25 (1H, d, J=12.0 Hz), 1.42 (3H, s), 1.39 (3H, s), 0.99 (3H, s), 0.97 (3H, s). ¹³C NMR (CDCl₃, 125 MHz) δ: 168.9, 163.6, 142.4, 140.3, 138.9, 136.8, 99.4, 93.8, 77.2, 71.1, 33.3, 31.9, 21.7, 18.9, 18.5. 19F NMR (CDCl₃, 470 MHz) δ: −151.6, −158.6, −162.7. HRMS (ESI) calcd for C₁₈H₁₈F₅NO₅ M+: m/z 423.1105, found m/z 423.1080.

### Perfluorophenyl (R)-3-(2,2,5,5-tetramethyl-1,3-dioxane-4-carboxamido)propanoate

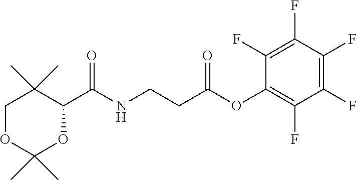 [SMILES: CC1(C)OCC(C)(C)[C@H](C(=O)NCCC(=O)Oc2c(F)c(F)c(F)c(F)c2F)O1]

To a 10 mL flask were added (R)-3-(2,2,5,5-tetramethyl-1,3-dioxane-4-carboxamido)propanoic acid (150 mg, 0.57 mmol), 2,3,4,5,6-pentafluorophenol (141 mg, 0.76 mmol), N-(3-dimethylaminopropyl)-N′-ethylcarbodiimide hydrochloride (132 mg, 0.68 mmol), 4-(dimethylamino)pyridine (3 mg, 0.02 mmol) and CH₂Cl₂ (4 mL). The resulting mixture was stirred at r.t. for 18 h, after which the solvent was reduced in vacuo. The crude residue was purified by flash column chromatography (petroleum ether-ethyl acetate (0-40%)) to yield the desired ester as a white solid (200 mg, 81%).

¹H NMR (CDCl₃, 500 MHz) δ: 7.01 (1H, brs), 4.13 (1H, s), 3.78-3.60 (2H, m), 3.71 (1H, d, J=12.5 Hz), 3.31 (1H, d, J=11.5 Hz), 2.98 (2H, t, J=6.5 Hz), 1.47 (3H, s), 1.45 (3H, s), 1.08 (3H, s), 1.00 (3H, s). ¹³C NMR (CDCl₃, 100 MHz) δ: 170.1, 168.1, 99.1, 77.2, 71.4, 34.1, 33.5, 33.0, 29.3, 22.0, 18.7, 18.6. ¹⁹F NMR (CDCl₃, 470 MHz) δ: −152.6, −157.6, −162.0. HRMS (ESI) calcd for C₁₈H₂₀F₅NCO₅ M+: m/z 425.1262, found m/z 425.1237.

### (R,E)-N-(3-((3-(2,5-Dioxo-2,5-dihydro-1H-pyrrol-1-yl)propyl)amino)-3-oxoprop-1-en-1-yl)-2,2,5,5-tetramethyl-1,3-dioxane-4-carboxamide

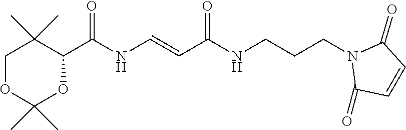 [SMILES: CC1(C)OCC(C)(C)[C@H](C(=O)N/C=C/C(=O)NCCCN2C(=O)C=CC2=O)O1]

To a 10 mL flask were added perfluorophenyl (R,E)-3-(2,2,5,5-tetramethyl-1,3-dioxane-4-carboxamido)acrylate (25 mg, 0.06 mmol), 3-(2,5-dioxo-2,5-dihydro-1H-pyrrol-1-yl)propan-1-aminium 2,2,2-trifluoroacetate salt (14.5 mg, 0.05 mmol) and CH₂Cl₂ (1.5 mL). The mixture was stirred for 10 min at r.t. before the addition of DIPEA (10 μL, 0.06 mmol). Stirring was continued for 20 h at r.t. before the mixture was reduced in vacuo, and the residue purified by flash column chromatography (CH₂Cl₂:CH₃OH (0-4%)), to obtain the desired product as pink solid (8 mg, 48%) (together with recovered of starting material (5 mg)).

¹H NMR (CDCl₃, 400 MHz) δ: 8.23 (1H, d, J=10.8 Hz), 7.77 (1H, dd, J=11.2, 11.2 Hz), 6.65 (2H, s), 5.95 (1H, brs), 5.75 (1H, d, J=14 Hz), 4.12 (1H, s), 3.64 (1H, d, J=11.6 Hz), 3.52 (2H, t, J=6 Hz), 3.24 (1H, d, J=11.6 Hz), 3.22-3.17 (2H, m), 1.76-1.70 (2H, m), 1.44 (3H, s), 1.38 (3H, s), 0.98 (3H, s), 0.93 (3H, s). ¹³C NMR (CDCl₃, 100 MHz) δ: 171.1, 168.0, 162.9, 134.2, 123.0, 105.9, 99.4, 77.2, 71.3, 35.9, 34.7, 33.4, 29.4, 28.3, 21.9, 18.8, 18.7. IR vₘax (neat)/cm⁻¹: 3323, 3050, 2926, 1705, 1664, 1600, 1097. HRMS (EI⁺) calcd for C₁₉H₂₇N₃O₆ M+: m/z 393.1900, found m/z 393.1903. [α]_{D} +54.667 (c=0.3, CHCl₃, T=23.9° C.).

### (R,E)-N-(3-((5-(2,5-Dioxo-2,5-dihydro-1H-pyrrol-1-yl) pentyl) amino)-3-oxoprop-1-en-1-yl)-2,2,5,5-tetramethyl-1,3-dioxane-4-carboxamide

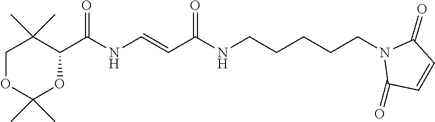 [SMILES: CC1(C)OCC(C)(C)[C@H](C(=O)N/C=C/C(=O)NCCCCCN2C(=O)C=CC2=O)O1]

To a 10 mL flask were added perfluorophenyl (R,E)-3-(2,2,5,5-tetramethyl-1,3-dioxane-4-carboxamido)acrylate (60 mg, 0.14 mmol), 1-(5-aminopentyl)-1H-pyrrole-2,5-dione trifluoroacetate salt (38 mg, 0.12 mmol) and CH₂Cl₂ (2.2 mL). The mixture was stirred for 10 min at r.t. before the addition of DIPEA (20 μL, 0.12 mmol). Stirring was continued for a further 72 h at r.t. before the mixture was reduced in vacuo, and the concentrate purified by flash column chromatography (CH₂Cl₂:CH₃OH (0-4%)) to obtain the desired product as colorless oil (32 mg, 60%).

¹H NMR (CDCl₃, 400 MHz) δ: 8.21 (1H, d, J=10.4 Hz), 7.69 (1H, dd, J=10.8, 10.8 Hz), 6.63 (2H, s), 5.73 (1H, d, J=14 Hz), 5.47 (1H, brs), 4.11 (1H, s), 3.63 (1H, d, J=11.6 Hz), 3.45 (2H, t, J=6.8 Hz), 3.25-3.17 (2H, m), 3.2 (1H, d, J=11.6 Hz), 3.22-3.17 (2H, m), 1.76-1.70 (2H, m), 1.57-1.47 (4H, m), 1.43 (3H, s), 1.38 (3H, s), 1.30-1.27 (2H, m), 0.98 (3H, s), 0.93 (3H, s). ¹³C NMR (CDCl₃, 100 MHz) δ: 170.9, 168.0, 166.2, 134.0, 132.7, 106.2, 99.4, 77.2, 71.3, 39.3, 37.4, 33.3, 29.4, 28.9, 28.2, 23.8, 21.9, 18.8, 18.7. IR νₘₐₓ (neat)/cm⁻¹: 3329, 3050, 2933, 1701, 1662, 1097, 720. HRMS (ESI) calcd for C₂₁H₃₁N₃O₆ M+: m/z 421.2213, found m/z 421.2207. [α]_{D} +25.09 (c=2.2, CHCl₃, T=23.0° C.).

### (R,Z)—N-(3-((3-(2,5-Dioxo-2,5-dihydro-1H-pyrrol-1-yl)propyl)amino)-3-oxoprop-1-en-1-yl)-2,2,5,5-tetramethyl-1,3-dioxane-4-carboxamide

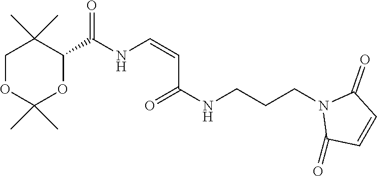 [SMILES: CC1(C)OCC(C)(C)[C@H](C(=O)N/C=C\C(=O)NCCCN2C(=O)C=CC2=O)O1]

To a 10 mL flask were added perfluorophenyl (R,Z)-3-(2,2,5,5-tetramethyl-1,3-dioxane-4-carboxamido)acrylate (25 mg, 0.05 mmol), 3-(2,5-dioxo-2,5-dihydro-1H-pyrrol-1-yl)propan-1-aminium 2,2,2-trifluoroacetate salt (14.5 mg, 0.05 mmol) and CH₂Cl₂ (1.4 mL). The resulting mixture was stirred for 10 min at r.t. before the addition of DIPEA (10 μL, 0.06 mmol). The reaction was stirred at r.t. for 72 h before the mixture was reduced under reduced pressure, and the crude residue was purified by flash column chromatography (CH₂Cl₂:CH₃OH (0-3%)), to obtain the desired product as yellow oil (4 mg, 17%).

¹H NMR (CDCl₃, 400 MHz) δ: 11.58 (1H, d, J=11.2 Hz), 7.25-7.20 (1H, dd, J=9.2, 9.2 Hz), 6.65 (2H, s), 5.89 (1H, t, J=6 Hz), 4.98 (1H, d, J=8.0 Hz), 4.12 (1H, s), 3.65 (1H, d, J=11.6 Hz), 3.53 (2H, t, J=6.4 Hz), 3.25 (1H, d, J=11.6 Hz), 3.23-3.13 (2H, m), 1.77-1.70 (2H, m), 1.53 (3H, s), 1.39 (3H, s), 0.98 (6H, s). ¹³C NMR (CDCl₃, 100 MHz) δ: 171.0, 168.8, 167.8, 134.2, 133.4, 100.5, 99.2, 77.2, 71.4, 33.5, 34.8, 33.1, 29.3, 28.3, 22.0, 19.0, 18.6. IR νₘₐₓ (neat)/cm⁻¹: 3311, 3050, 2928, 1705, 1654, 1097, 696. HRMS (EI) calcd for C₁₉H₂₇N₃O₆ M+: m/z 393.1900, found m/z 393.1898. [α]_{D} +24.00 (c=0.5, CHCl₃, T=23.8° C.).

### (R,Z)—N-(3-((5-(2,5-Dioxo-2,5-dihydro-1H-pyrrol-1-yl) pentyl) amino)-3-oxoprop-1-en-1-yl)-2,2,5,5-tetramethyl-1,3-dioxane-4-carboxamide

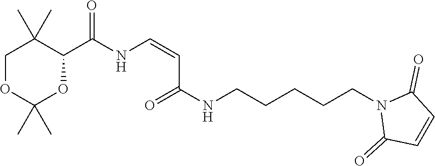 [SMILES: CC1(C)OCC(C)(C)[C@H](C(=O)N/C=C\C(=O)NCCCCCN2C(=O)C=CC2=O)O1]

To a 10 mL flask were added perfluorophenyl (R,Z)-3-(2,2,5,5-tetramethyl-1,3-dioxane-4-carboxamido)acrylate (30 mg, 0.07 mmol), 1-(5-aminopentyl)-1H-pyrrole-2,5-dione trifluoroacetate salt (15 mg, 0.05 mmol) and CH₂Cl₂ (1.2 mL). The reaction mixture was stirred for 10 min at r.t. before the addition of DIPEA (10 μL, 0.06 mmol). Stirring was continued at r.t. for 72 h before the mixture was reduced in vacuo, and the crude concentrate purified by flash column chromatography (CH₂Cl₂:CH₃OH (0-3%)), to yield the desired product as colorless oil (12 mg, 40%).

¹H NMR (CDCl₃, 400 MHz) δ: 11.59 (1H, d, J=11.2 Hz), 7.23-7.18 (1H, dd, J=4.0, 2.0 Hz), 6.63 (2H, s), 5.37 (1H, t, J=5.2 Hz), 4.92 (1H, d, J=9.2 Hz), 4.12 (1H, s), 3.64 (1H, d, J=11.6 Hz), 3.46 (2H, t, J=6.8 Hz), 3.25 (1H, d, J=11.6 Hz), 3.22-3.19 (2H, m), 1.58-1.43 (4H, m), 1.53 (3H, s), 1.37 (3H, s), 1.32-1.21 (2H, m), 1.18 (3H, s), 0.99 (3H, s). ¹³C NMR (CDCl₃, 100 MHz) δ: 170.9, 169.4, 168.2, 134.1, 133.2, 100.9, 99.2, 77.2, 71.4, 38.9, 37.3, 33.3, 29.7, 28.7, 28.1, 23.8, 22.0, 19.0, 18.6. HRMS (ESI) calcd for C₂₁H₃₁N₃O₆ M+: m/z 421.2213, found m/z 421.2189. IR νₘₐₓ (neat)/cm⁻¹: 3323, 2926, 2854, 1703, 1656, 1465, 1097, 720. [α]_{D} +12.5 (c=0.8, CHCl₃, T=23.9° C.).

### (R)—N-(3-((3-(2,5-Dioxo-2,5-dihydro-1H-pyrrol-1-yl)propyl)amino)-3-oxopropyl)-2,2,5,5-tetramethyl-1,3-dioxane-4-carboxamide

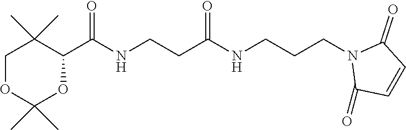 [SMILES: CC1(C)OCC(C)(C)[C@H](C(=O)NCCC(=O)NCCCN2C(=O)C=CC2=O)O1]

To a 25 mL flask were added perfluorophenyl (R)-3-(2,2,5,5-tetramethyl-1,3-dioxane-4-carboxamido)propanoate (95 mg, 0.22 mmol), 3-(2,5-dioxo-2,5-dihydro-1H-pyrrol-1-yl)propan-1-aminium 2,2,2-trifluoroacetate salt (80 mg, 0.29 mmol) and CH₂Cl₂ (8 mL). The mixture was then stirred at r.t. for 10 min before the addition of DIPEA (70 μL, 0.42 mmol). Stirring was continued for a further 72 h at room temperature before the mixture was reduced in vacuo. The crude concentrate was then purified by flash column chromatography (CH₂Cl₂:Methanol (0-3%)) to obtain the desired product as colorless oil (52 mg, 59%).

¹H NMR (CDCl₃, 500 MHz) δ: 7.00 (1H, t, J=7 Hz), 6.65 (2H, s), 6.32 (1H, t, J=7 Hz), 4.01 (1H, s), 3.67 (1H, d, J=11.6 Hz), 3.55-3.42 (2H, m), 3.50 (2H, t, J=8 Hz), 3.21 (1H, d, J=15 Hz), 3.16-3.11 (2H, m), 2.39 (2H, t, J=7.5 Hz), 1.74-1.68 (2H, m), 1.38 (3H, s), 1.34 (3H, s), 0.96 (3H, s), 0.90 (3H, s). ¹³C NMR (CDCl₃, 125 MHz) δ: 171.1, 170.9, 170.0, 134.2, 99.0, 77.2, 71.4, 36.1, 36.0, 34.8, 32.9, 30.9, 29.4, 28.2, 22.1, 18.8, 18.6. IR νₘₐₓ (neat)/cm⁻¹: 3310, 3098, 2945, 1706, 1647, 1533, 1097, 696. HRMS (ESI) calcd for C₁₉H₂₉N₃O₆ M+: m/z 395.2056, found m/z 395.2033. [α]_{D} +21.231 (c=1.3, CHCl₃, T=23.7° C.).

### (R)—N-(3-((5-(2,5-Dioxo-2,5-dihydro-1H-pyrrol-1-yl)pentyl)amino)-3-oxopropyl)-2,2,5,5-tetramethyl-1,3-dioxane-4-carboxamide

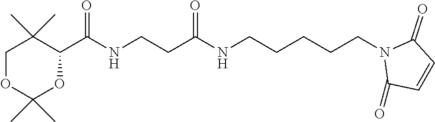 [SMILES: CC1(C)OCC(C)(C)[C@H](C(=O)NCCC(=O)NCCCCCN2C(=O)C=CC2=O)O1]

To a 25 mL flask were added perfluorophenyl (R)-3-(2,2,5,5-tetramethyl-1,3-dioxane-4-carboxamido)propanoate (95 mg, 0.22 mmol), 1-(5-aminopentyl)-1H-pyrrole-2,5-dione trifluoroacetate salt (60 mg, 0.20 mmol) and CH₂Cl₂ (5 mL). The mixture was stirred for 10 min at r.t. before the addition of DIPEA (60 μL, 0.36 mmol). Stirring was continued for a further 72 h at r.t. before the mixture was reduced in vacuo. The crude residue was purified by flash column chromatography (CH₂Cl₂:CH₃OH, (0-3%)), to obtain the desired adduct as colorless oil (90 mg, 95%).

¹H NMR (CDCl₃, 500 MHz) δ: 6.97 (1H, t, J=5.5 Hz), 6.63 (2H, s), 5.98 (1H, brs), 4.00 (1H, s), 3.61 (1H, d, J=11.5 Hz), 3.58-3.46 (2H, m), 3.44 (2H, t, J=7.0 Hz), 3.21 (1H, d, J=11.5 Hz), 3.17-3.12 (2H, m), 2.36 (2H, t, J=6.0 Hz), 1.56-1.42 (4H, m), 1.39 (3H, s), 1.34 (3H, s), 1.26-1.18 (2H, m), 1.03 (3H, s), 0.80 (3H, s). ¹³C NMR (CDCl₃, 125 MHz) δ: 170.9, 170.8, 170.2, 134.2, 99.0, 77.2, 71.4, 39.3, 37.4, 36.1, 34.9, 32.9, 29.4, 29.2, 28.1, 23.9, 22.1, 18.8, 18.6. IR vₘax (neat)/cm⁻¹: 3330, 2930, 1706, 1656, 1521, 1097, 696. HRMS (ESI) calcd for C₂₁H₃₃N₃O₆ M+: m/z 423.2369, found m/z 423.2364. [α]_{D} +24.267 (c=1.5, CHCl₃, T=23.7° C.).

### (R,E)-N-(3-((3-(2,5-Dioxo-2,5-dihydro-1H-pyrrol-1-yl)propyl)amino)-3-oxoprop-1-en-1-yl)-2,4-dihydroxy-3,3-dimethylbutanamide

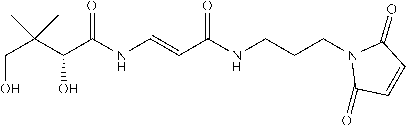 [SMILES: CC(C)(CO)[C@@H](O)C(=O)N/C=C/C(=O)NCCCN1C(=O)C=CC1=O]

To a 5 mL flask were added (R,E)-N-(3-((3-(2,5-dioxo-2,5-dihydro-1H-pyrrol-1-yl)propyl)amino)-3-oxoprop-1-en-1-yl)-2,2,5,5-tetramethyl-1,3-dioxane-4-carboxamide (12 mg, 0.03 mmol), CH₃CN (0.2 mL), bismuth chloride (1 mg, 0.003 mmol) and H₂O (10 μL). The reaction mixture was stirred for 24 h at r.t. and was then quenched with saturated aqueous NaHCO₃. The mixture was subsequently diluted with ethyl acetate before being filtered through Celite®. The solvent was removed in vacuo, and the crude residue was purified by flash column chromatography (CH₂Cl₂:Methanol:NH₃ (0-5%)) to afford the desired product as colorless oil (10 mg, 92%).

¹H NMR ((CD₃)₂CO), 400 MHz) δ: 9.56 (1H, brs), 7.86 (1H, dd, J=12.0, 11.2 Hz), 7.08 (1H, brs), 6.87 (2H, s), 6.00 (1H, d, J=13.6 Hz), 5.17 (1H, d, J=7.0 Hz), 3.95 (2H, d, J=5.2 Hz), 3.38 (2H, t, J=6.8 Hz), 3.24 (1H, d, J=11.6 Hz), 3.12-3.11 (2H, m), 1.64-1.61 (2H, m), 1.16 (6H, s). ¹³C NMR ((CD₃)₂CO), 100 MHz) δ: 170.8, 134.2, 133.0, 105.4, 76.5, 69.4, 39.3, 36.4, 35.2, 28.9, 20.4, 19.7. IR νₘₐₓ (neat)/cm⁻¹: 3323, 2924, 1701, 1654, 1329, 1188. HRMS (ESI) calcd for C₁₆H₂₃N₃O₆ M+: m/z 353.1587, found m/z 353.1562. [α]_{D} +16.8 (c=0.5, acetone, T=24.0° C.).

### (R,E)-N-(3-((5-(2,5-Dioxo-2,5-dihydro-1H-pyrrol-1-yl) pentyl) amino)-3-oxoprop-1-en-1-yl)-2,4-dihydroxy-3,3-dimethylbutanamide

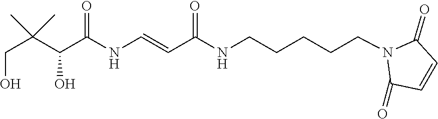 [SMILES: CC(C)(CO)[C@@H](O)C(=O)N/C=C/C(=O)NCCCCCN1C(=O)C=CC1=O]

In a 5 mL flask were combined (R,E)-/V-(3-((5-(2,5-dioxo-2,5-dihydro-1H-pyrrol-1-yl)pentyl)amino)-3-oxoprop-1-en-1-yl)-2,2,5,5-tetramethyl-1,3-dioxane-4-carboxamide (10 mg, 0.02 mmol), CH₃CN (0.2 mL), bismuth chloride (1 mg, 0.003 mmol) and H₂O (10 μL). The reaction mixture was then stirred at r.t. for 24 h and then quenched with saturated aqueous NaHCO₃. The mixture was diluted with ethyl acetate and filtered through Celite®. The solvent was removed in vacuo, and the crude residue was purified by flash column chromatography (CH₂Cl₂:Methanol:NH₃ (0-5%)) to afford the desired product as colorless oil (3 mg, 33%).

HRMS (ESI) calcd for C₁₈H₂₇N₃O₆ M+: m/z 381.1900, found m/z 381.1880.

### (R,Z)—N-(3-((3-(2,5-Dioxo-2,5-dihydro-1H-pyrrol-1-yl)propyl)amino)-3-oxoprop-1-en-1-yl)-2,4-dihydroxy-3,3-dimethylbutanamide

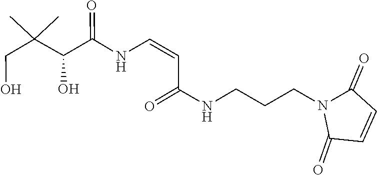 [SMILES: CC(C)(CO)[C@@H](O)C(=O)N/C=C\C(=O)NCCCN1C(=O)C=CC1=O]

To a 5 mL flask were added (R,Z)—N-(3-((3-(2,5-dioxo-2,5-dihydro-1H-pyrrol-1-yl)propyl)amino)-3-oxoprop-1-en-1-yl)-2,2,5,5-tetramethyl-1,3-dioxane-4-carboxamide (18 mg, 0.05 mmol), CH₃CN (0.3 mL), bismuth chloride (3 mg, 0.009 mmol) and H₂O (30 μL). The reaction was stirred at r.t. for 24 h, and was then treated with a few drops of saturated aqueous NaHCO₃ and subsequently diluted with ethyl acetate. The solution was filtered through Celite® and the solvent was removed in vacuo. Purification of the crude residue by flash column chromatography (CH₂Cl₂:Methanol:NH₃ (0-5%)) afforded the desired adduct obtained as colorless oil (5 mg, 30%).

¹H NMR ((CD₃)₂CO, 400 MHz) δ: 11.85 (1H, d, J=8.4 Hz), 7.12-7.07 (1H, dd, J=9.2, 8.8 Hz), 6.73 (2H, s), 5.07 (1H, d, J=6.0 Hz), 3.98 (1H, s), 3.42-3.30 (3H, m), 3.29 (1H, d, J=10.8 Hz), 3.09-3.04 (2H, m), 1.75-1.66 (2H, m), 1.16 (6H, s). ¹³C NMR ((CD₃)₂CO), 100 MHz) δ: 170.8, 134.2, 133.0, 100.1, 76.5, 69.4, 39.3, 36.1, 35.1, 28.9, 20.4, 19.7. HRMS (ESI) calcd for C₁₆H₂₃N₃O₆ M+: m/z 353.1587, found m/z 353.1567.

### (R,Z)—N-(3-((5-(2,5-Dioxo-2,5-dihydro-1H-pyrrol-1-yl) pentyl) amino)-3-oxoprop-1-en-1-yl)-2,4-dihydroxy-3,3-dimethylbutanamide

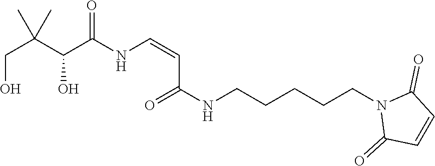 [SMILES: CC(C)(CO)[C@@H](O)C(=O)N/C=C\C(=O)NCCCCCN1C(=O)C=CC1=O]

To a 5 mL flask were added (R,Z)—N-(3-((5-(2,5-dioxo-2,5-dihydro-1H-pyrrol-1-yl)pentyl)amino)-3-oxoprop-1-en-1-yl)-2,2,5,5-tetramethyl-1,3-dioxane-4-carboxamide (19 mg, 0.04 mmol), CH₃CN (0.3 mL ), BiCl₃ (3 mg, 0.009 mmol) and H₂O (30 μL). The mixture was stirred at r.t. for 24 h and was treated with a few drops of saturated aqueous NaHCO₃. The mixture was diluted with ethyl acetate and then filtered through Celite®. The solvent was removed in vacuo, and the crude residue was purified by flash column chromatography (CH₂Cl₂:Methanol:NH₃ (0-5%)) to give the desired product as colorless oil (5 mg, 29%).

HRMS (ESI) calcd for C₁₈H₂₇N₃O₆ M+: m/z 381.1900, found m/z 381.1877.

### (R)—N-(3-((5-(2,5-Dioxo-2,5-dihydro-1H-pyrrol-1-yl)pentyl)amino)-3-oxopropyl)-2,4-di hydroxy-3, 3-dimethylbutanamide

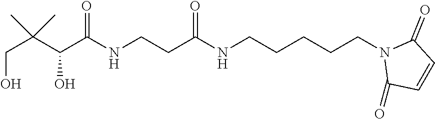 [SMILES: CC(C)(CO)[C@@H](O)C(=O)NCCC(=O)NCCCCCN1C(=O)C=CC1=O]

To a 5 mL flask were added (R)—N-(3-((5-(2,5-dioxo-2,5-dihydro-1H-pyrrol-1-yl)pentyl)amino)-3-oxopropyl)-2,2,5,5-tetramethyl-1,3-dioxane-4-carboxamide (100 mg, 0.23 mmol), CH₃CN (1.6 mL ), BiCl₃ (8 mg, 0.02 mmol) and H₂O (80 μL). The mixture was stirred at r.t. for 24 h and then treated with a few drops of saturated aqueous NaHCO₃. The mixture was subsequently diluted with ethyl acetate and filtered through Celite®. The solvent was removed in vacuo and the crude residue was purified by flash column chromatography (CH₂Cl₂:Methanol:NH₃ (0-5%)) to afford the desired product as a yellow oil (43 mg, 47%).

¹H NMR ((CD₃)₂CO), 500 MHz) δ: 7.52 (1H, t, J=6.0 Hz), 7.22 (1H, br s), 6.73 (2H, s), 3.80 (1H, s), 3.41-3.24 (6H, m), 3.06-3.02 (2H, m), 2.28 (2H, t, J=5.5 Hz), 1.46-1.34 (4H, m), 1.20-1.18 (2H, m), 0.86 (3H, s), 0.74 (3H, s). ¹³C NMR (CDCl₃, 125 MHz) δ: 173.6, 170.9, 170.8, 134.2, 76.3, 69.6, 63.3, 39.2, 38.8, 37.1, 35.2, 29.4, 28.0, 23.8, 21.1, 19.8. IR uₘₐₓ (neat)/cm⁻¹: 3310, 2931, 1700, 1646, 1539, 1410, 695. HRMS (ESI) calcd for C₁₈H₂₉N₃O₆ M+: m/z 383.2056, found m/z 383.2051. [α]_{D} +13.023 (c=4.3, acetone, T=23.9° C.).

### Compound Syntheses—Ivermectin Conjugate

### 5-O-(tert-Butyldimethylsilyl)avermectin B1a

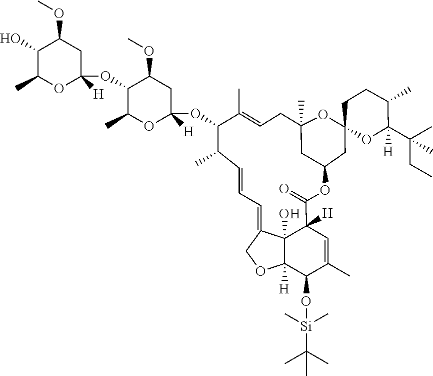

A solution of ivermectin B1a (980 mg, 1.1 mmol) in anhydrous DMF (8.0 mL ) was treated with imidazole (495 mg, 7.3 mmol), followed by a solution of TBDMSCl (560 mg, 3.28 mmol) in anhydrous DMF (2.0 mL ). The reaction mixture was stirred at rt for 2.5 h and was then diluted with Et₂O (25 mL ) followed by H₂O (15 mL ). The resulting emulsion was then stirred for 0.5 h, the organic layer was separated, and aqueous phase was extracted with Et₂O (3×25 mL ). The combined organics were washed with H₂O (5×100 mL ) and brine (2×100 mL ). The combined organic washes were dried over Na₂SO₄ and concentrated under reduced pressure. Purification of the crude residue by flash column chromatography (silica gel, elution gradient 0-30% EtOAc in petroleum ether) yielded the expected product as a white solid (638 mg, 58%).

¹H NMR (CDCl₃, 400 MHz) δ: 5.83-5.78 (1H, br m), 5.73-5.69 (2H, br m), 5.38 (1H, d, J=3.2 Hz), 5.32 (1H, br s), 5.29-5.28 (1H, br m), 4.97 (1H, d, J=9.6 Hz), 4.76 (1H, d, J=3.2 Hz), 4.67 (1H, d, J=14.4 Hz), 4.56 (1H, d, J=14.8 Hz), 4.42 (1H, brs), 3.92 (1H, brs), 3.85-3.78 (2H, br m), 3.77-3.72 (1H, br m,), 3.68-3.57 (2H, br m), 3.50-3.45 (1H, br m), 3.41 (3H, s), 3.40 (3H, s), 3.36 (1H, brs), 3.25-3.19 (2H, br m), 3.14 (1H, t, J=8.8 Hz), 2.53 (1H, app t, J=6.8 Hz), 2.35-2.29 (2H, br m), 2.28-2.25 (1H, brm), 2.21 (1H, dd, J=13.2, 5.0 Hz), 1.98 (1H, dd, J=12.0, 4.4 Hz), 1.77 (3H, s), 1.74-1.71 (1H, brm), 1.64 (1H, d, J=11.1 Hz), 1.59-1.52 (7H, br m), 1.49 (3H, s), 1.47-1.39 (2H, br m), 1.33 (1H, t, J=11.6 Hz), 1.26 (3H, br s), 1.25 (3H, brs), 1.14 (3H, d, J=6.8 Hz), 0.96-0.92 (3H, brm), 0.91 (9H, s), 0.87-0.81 (4H, br m), 0.76 (3H, d, J=4.4 Hz), 0.12 (6H, s). ¹³C NMR (CDCl₃, 100 MHz) δ: 174.0, 140.2, 137.5, 137.4, 135.0, 124.8, 119.3, 118.3, 117.3, 98.4, 97.4, 94.7, 81.8, 80.3, 80.2, 80.0, 79.3, 78.1, 76.5, 76.0, 69.4, 68.6, 68.1, 67.9, 67.2, 56.4, 56.3, 45.7, 41.1, 39.6, 36.8, 35.7, 35.4, 34.5, 34.1, 31.2, 27.3, 26.8, 25.8, 20.2, 20.0, 18.4, 17.6, 17.4, 15.1, 12.4, 12.0, −4.6, −4.8.

### 4″-O—[(R,Z)-3-(2,2,5,5-Tetramethyl-1,3-dioxane-4-carboxamido)acrylate]-5-O-(tert-butyldimethylsilyl)avermectin B1a

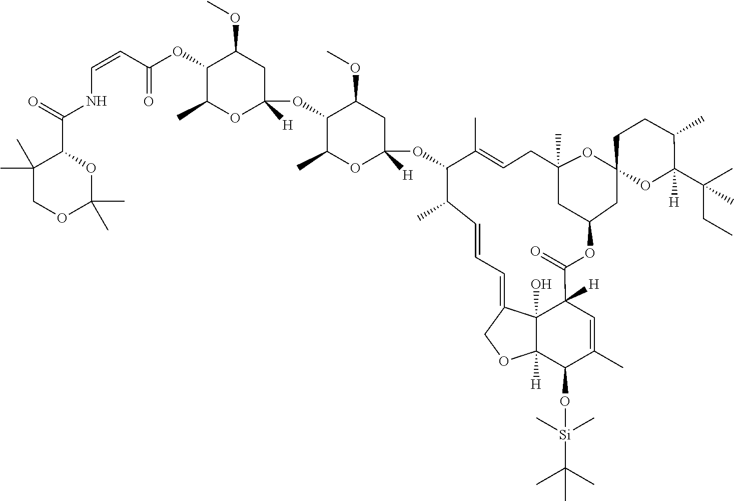

A solution of 5-O-(tert-butyldimethylsilyl)avermectin B1a (45 mg, 40 μmol) in CH₂Cl₂ (1 mL) was treated sequentially with DCC (19 mg, 90 μmol), followed by DMAP (10 mg, 80 μmol) and a solution of (R,Z)-3-(2,2,5,5-tetramethyl-1,3-dioxane-4-carboxamido)acrylic acid (10 mg, 40 μmol) in CH₂Cl₂ (1.5 mL). The reaction mixture was stirred at rt until completion as indicated by TLC analysis (18 h). The reaction was then diluted with CH₂Cl₂ (5 mL). The organic phase was washed with 1M HCl (7 mL), followed by saturated aqueous NaHCO₃ (7 mL) and brine (7 mL). The organic layer was dried over Na₂SO₄ and concentrated under reduced pressure. Purification of the crude residue by flash column chromatography (silica gel, elution gradient 0-10% EtOAc in petroleum ether) afforded TBS protected ivermectin as a colourless oil (30 mg, 61%).

¹H NMR (CDCl₃, 400 MHz) δ: 11.12 (1H, d, J=11.6 Hz), 7.52-7.43 (1H, m), 5.87-5.82 (1H, brm), 5.79-5.71 (2H, brm), 5.42 (1H, brs), 5.36-5.31 (2H, brm), 5.19 (1H, d, J=8.9 Hz), 5.00 (1H, d, J=9.9 Hz), 4.79 (1H, brs), 4.77 (1H, appt, J=9.6 Hz), 4.70 (1H, d, J=14.6 Hz), 4.60 (1H, d, J=14.5 Hz), 4.46 (1H, br s), 4.22 (1H, s), 3.96 (1H, br s), 3.92-3.83 (3H, m), 3.73 (1H, d, J=11.7 Hz), 3.70-3.60 (3H, m), 3.45 (3H, s), 3.42-3.38 (4H, m), 3.34 (1H, d, J=11.7 Hz), 3.28-3.20 (2H, m), 2.53 (1H, br s), 2.38-2.32 (2H, m), 2.31-2.22 (2H, m), 2.00 (1H, dd, J=11.7, 3.8 Hz), 1.81 (3H, s), 1.79-1.62 (9H, m), 1.59 (3H, s), 1.47 (3H, s), 1.44-1.41 (5H, m), 1.37 (1H, t, J=12.4 Hz), 1.28 (3H, brs), 1.27 (3H, brs), 1.18 (3H, d, J=6.3 Hz), 1.07 (3H, s), 1.05 (3H, s), 0.99-0.96 (3H, m), 0.94 (9H, s), 0.88-0.85 (4H, m), 0.81-0.79 (1H, m), 0.15 (6H, s). ¹³C NMR (CDCl₃, 100 MHz) δ: 174.0, 168.9, 167.2, 140.3, 137.5, 137.4, 136.6, 135.0, 124.8, 119.3, 118.3, 117.2, 99.3, 98.4, 98.2, 97.5, 94.8, 81.9, 80.8, 80.2, 80.0, 79.2, 77.2, 77.1, 76.6, 75.7, 75.6, 71.3, 69.5, 68.7, 67.9, 67.1, 57.2, 56.5, 45.7, 41.1, 39.6, 36.8, 35.7, 35.4, 35.2, 34.5, 34.1, 33.3, 31.2, 29.3, 27.2, 26.9, 25.8, 21.9, 20.3, 20.0, 18.9, 18.6, 18.4, 17.4 (2C), 15.2, 12.4, 12.1, −4.5, −4.8. HRMS (ESI) calculated for C₆₈H₁₀₉NO₁₈Si [M−CO₂]⁺: m/z 1211.7516, found m/z 1211.6699. IR uₘₐₓ (film)/cm⁻¹: 3420, 2945,2879, 1775, 1670, 1550, 1392, 1128. [α]_{D} +12.21 (c=0.7, CHCl₃, T=23.1° C.).

### 4″-O—[(R,Z)-3-(2,2,5,5-Tetramethyl-1,3-dioxane-4-carboxamido)acrylate]avermectin B1a, 14

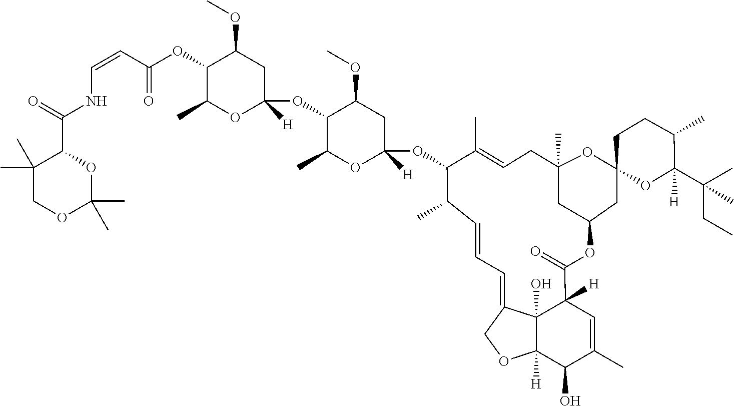

A solution of 4″-O—[(R,Z)-3-(2,2,5,5-tetramethyl-1,3-dioxane-4-carboxamido)acrylate]-5-O-(tert-butyldimethylsilyl)avermectin B1a (30 mg, 20 μmol) in MeOH (2.5 mL) was treated with a catalytic amount of p-TsOH (3 mg). The reaction mixture was stirred at 18° C. for 30 min. The reaction was then diluted with H₂O (17 mL) followed by EtOAc (20 mL). The organic layer was washed with H₂O (3×20 mL) and brine (20 mL). The organic phase was dried over Na₂SO₄ and concentrated under reduced pressure. Purification of the crude residue by flash column chromatography (silica gel, elution gradient 0-3% MeOH/CH₂Cl₂) afforded 4″-O—[(R,Z)-3-(2,2,5,5-Tetramethyl-1,3-dioxane-4-carboxamido)acrylate]avermectin B1a as a yellow oil (16 mg, 58%).

¹H NMR (CDCl₃, 400 MHz) δ: 11.27 (1H, d, J=11.6 Hz), 7.52 (1H, dd, J=11.6 Hz, 9.6 Hz), 5.89 (1H, d, J=9.2 Hz), 5.82-5.69 (2H, m), 5.44 (1H, br s), 5.42-5.32 (2H, m), 5.23 (1H, d, J=9.2 Hz), 5.00 (1H, d, J=10.0 Hz), 4.80 (1H, brs), 4.75 (1H, d, J=9.5 Hz), 4.72-4.65 (2H, m), 4.32 (1H, d, J=6.0 Hz), 4.23 (1H, s), 4.13 (1H, brs), 3.99 (2H, m), 3.92-3.84 (2H, m), 3.73-3.62 (4H, m), 3.58 (1H, d, J=6.8 Hz), 3.45 (3H, brs), 3.38 (3H, brs), 3.30 (1H, brs), 3.28-3.21 (3H, m), 2.54 (1H, br s), 2.39-2.32 (2H, m), 2.32-2.22 (2H, m), 2.00 (1H, dd, J=12.0, 4.4 Hz), 1.89 (3H, s), 1.77 (1H, d, J=11.6 Hz), 1.67 (1H, d, J=9.6 Hz), 1.62-1.47 (16H, m), 1.47-1.34 (3H, m), 1.29-1.25 (3H, m), 1.20-1.15 (6H, m), 1.07 (3H, s), 1.00 (3H, s), 0.95 (3H, t, J=7.2 Hz), 0.87 (3H, d, J=6.6 Hz), 0.83-0.78 (4H, m). ¹³C NMR (CDCl₃, 100 MHz) δ: 174.0, 168.9, 167.2, 140.3, 138.0, 137.9, 137.2, 135.0, 124.7, 120.4, 118.3, 118.0, 107.0, 98.5, 98.3, 97.4, 94.8, 81.8, 80.7, 80.4, 79.2, 79.0, 77.9, 77.2, 76.3, 75.8, 75.7, 71.3, 68.6, 68.5, 67.7, 67.2, 67.1, 57.1, 56.5, 45.7, 41.1, 39.7, 36.9, 35.7, 35.4, 35.1, 34.5, 34.1 (2C), 31.2, 28.0, 27.2, 22.9, 20.8, 20.5, 20.2, 20.0, 18.8, 18.4, 17.4 (2C), 15.1, 12.4, 12.1. HRMS (ESI) calculated for C₆₂H₉₅NO₁₈ [M−CO₂]⁺: m/z 1097.6651, found m/z 1097.1176. IR uₘₐₓ (film)/cm⁻¹: 3680, 2947, 2879, 1783, 1645, 1498, 1288. [α]D +4.50 (c=0.2, CHCl₃, T=23.0° C.).

### Compound Syntheses—BODIPY-Ivermectin Complex

### 5-(3-Azidopropyl)-1H-pyrrole-2-carbaldehyde

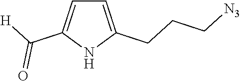 [SMILES: [N-]=[N+]=NCCCc1ccc(C=O)[nH]1]

Anhydrous DMF (20 mL ) was cooled down to 0° C. and was then treated with POCl₃ (0.6 mL , 6.6 mmol). The resulting solution was then stirred at 0° C. for 5 min, and then at rt for an additional 30 min. The reaction was then cooled back down to 0° C., and treated with a solution of 2-(3-azidopropyl)-1H-pyrrole (805 mg, 5.3 mmol) in anhydrous DMF (2 mL ). The mixture was then heated to 40° C. until completion by TLC analysis (18 h). The reaction was cooled back down to rt, and diluted with EtOAc (5 mL ), and treated with 4M aqueous NaOH solution (5 mL ). The phases were separated, and the aqueous layer was extracted with EtOAc (3×5 mL ). The combined organic layers were washed with H₂O (5×20 mL ), brine (2×20 mL ) and dried over Na₂SO₄. The resulting solution was concentrated under reduced pressure, to afford the desired aldehyde as a brown oil (490 mg, 51%). The product was used without further purification.

¹H NMR (CDCl₃, 400 MHz) δ: 9.95 (1H, brs), 9.41 (1H, s), 6.93-6.91 (1H, m), 6.13-6.12 (1H, m), 3.01 (2H, t, J=8.0 Hz), 2.72 (2H, t, J=7.0 Hz), 1.8-1.6 (2H, m). ¹³C NMR (CDCl₃, 100 MHz) δ: 178.3, 129.7, 128.5, 127.8, 109.6, 60.4, 28.0, 24.4. HRMS (ESI) calculated for C₈H₁₀N₄O [M]+: m/z 178.0855, found m/z 178.0851. IR umax (film)/cm⁻¹: 3233, 2831,2099, 1735, 1653, 1376.

### tert-Butyl (E)-3-(1H-pyrrol-2-yl)acrylate

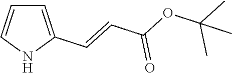

A solution of pyrrole-2-carboxaldehyde (1.5 g, 15.2 mmol) in benzene (110 mL ) was treated with (tert-butoxycarbonylmethylene)triphenylphosphorane (10.0 g, 26.5 mmol). The reaction mixture was heated to 80° C. until completion by TLC analysis (22 h). The reaction was then cooled to rt, and concentrated under reduced pressure. Purification of the crude residue by flash column chromatography (0-20% EtOAc/PE) afforded the expected ester as an orange oil (2.45 g, 83%).

¹H NMR (CDCl₃, 400 MHz) δ: 9.00 (1H, brs), 7.49 (1H, d, J=16.0 Hz), 6.92 (1H, apps), 6.55 (1H, apps), 6.29 (1H, apps), 6.01 (1H, d, J=16.0 Hz), 1.55 (9H, s). ¹³C NMR (CDCl₃, 100 MHz) δ: 167.1, 133.3, 128.5, 122.0, 113.7, 113.3, 110.8, 80.2, 28.2. HRMS (ESI) calculated for C₁₁H₁₅NO₂ [M+Na]⁺: m/z 216.0995, found m/z 216.0966. IR uₘₐₓ (film)/cm⁻¹: 3358, 2919, 2848, 1713, 1632, 1150.

### tert-Butyl 3-(1H-pyrrol-2-yl)propanoate

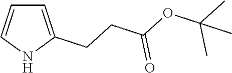

A solution of (E)-3-(1H-pyrrol-2-yl)acrylate (2.4 g, 12.4 mmol) in anhydrous MeOH (95 mL ) was stirred for 5 minutes under an argon atmosphere. Pd/C (10%, 160 mg, 7 mol %) was added to the solution, and the reaction placed under a hydrogen atmosphere, and stirred at rt until completion by TLC analysis (18 h). The reaction was filtered through a Celite® bed, and washed with MeOH (2×25 mL ). The organic phases were combined, and concentrated in vacuo to afford the desired ester as a brown oil (2.23 g, 92%). The product obtained required no further purification.

¹H NMR (CDCl₃, 400 MHz) δ: 8.62 (1H, brs), 6.69 (1H, apps), 6.12 (1H, appd, J=2.4 Hz), 5.93 (1H, apps), 2.89 (2H, t, J=6.4 Hz), 2.57 (2H, t, J=6.4 Hz), 1.47 (9H, s). ¹³C NMR (CDCl₃, 100 MHz) δ: 173.6, 131.3, 116.7, 107.8, 105.4, 80.7, 35.7, 28.1, 22.6. HRMS (ESI) calculated for C₁₁H₁₇NO₂ [M+Na]⁺: m/z 218.1151, found m/z 218.1168. IR uₘₐₓ (film)/cm⁻¹: 3394, 2919, 2848, 1644.

### tert-Butyl 3-(5-(3-azidopropyl)-4,4-difluoro-4-bora-3a,4a-diaza-s-indacene-3-yl)propanoate

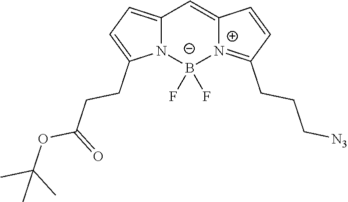 [SMILES: CC(C)(C)OC(=O)CCc1ccc2n1[B-](F)(F)[N+]1=C(CCCN=[N+]=[N-])C=CC1=C2]

A 0° C. solution of 5-(3-azidopropyl)-1H-pyrrole-2-carbaldehyde (330 mg, 1.9 mmol) in CH₂Cl₂ (8 mL ), was treated with a solution tert-butyl 3-(1H-pyrrol-2-yl)propanoate (346 mg, 1.8 mmol) in CH₂Cl₂ (2 mL ). The resulting mixture was then treated by the dropwise addition of POCl₃ (150 μL, 1.5 mmol), and was then stirred at rt for 6.5 h before being cooled back down to 0° C. The reaction mixture was then treated sequentially with BF₃.Et₂O (0.9 mL , 7.2 mmol) followed by N,N-diisopropylethylamine (1.5 mL , 8.5 mmol). The mixture was then stirred at rt until completion by TLC analysis (18 h). The reaction was quenched with H₂O (10 mL), diluted with CH₂Cl₂ (5 mL) and filtered through a bed of Celite®. The Celite® was washed with CH₂Cl₂ (2×10 mL), and the organic phases combined. The combined organic washes were dried over Na₂SO₄, and concentrated under reduced pressure. Purification of the crude residue by flash column chromatography (silica gel, elution gradient 0-15% EtOAc in petroleum ether) yielded the expected ester as a red oil (71 mg, 9%).

¹H NMR (CDCl₃, 400 MHz) δ: 7.13 (1H, s), 7.00-6.92 (2H, m), 6.37-6.34 (2H, m), 3.35-3.27 (4H, m), 2.80 (2H, t, J=8.0 Hz), 2.67 (2H, t, J=8.0 Hz), 1.46 (9H, s), 1.27 (2H, appt, J=7.2 Hz). ¹³C NMR (CDCl₃, 100 MHz) δ: 172.7, 161.6, 161.1, 134.7, 134.6, 130.6, 130.2, 127.8, 118.7, 118.1, 80.7, 51.8, 34.2, 32.8, 29.7, 28.2, 24.2. ¹⁹F NMR (CDCl₃, 376 MHz) δ: −144.17, −144.26, −144.35, −144.44. HRMS (ESI) calculated for C₁₉H₂₄BF₂N₅O₂ [M−N₃]⁺: m/z 361.1899, found m/z 361.3293. IR uₘₐₓ (film)/cm⁻¹: 3172, 2978, 2150, 1733, 1609, 1490, 1439, 1155.

λ_{exc}=467 nm. (at 510 nm emission, c=0.2 nM, MeOH).

λₑₘᵢₛ=516 nm. (at 315 nm excitation, c=0.2 nM, MeOH).

### Methyl 3-(5-formyl-1H-pyrrol-2-yl)propanoate

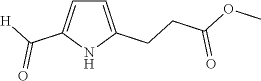

Anhydrous DMF (10 mL ) was cooled down to 0° C. and was treated with POCl₃ (0.6 mL , 6.6 mmol). The resulting solution was then stirred at 0° C. for 5 min, and then at rt for an additional 30 min. The reaction was cooled back down to 0° C., and treated with a solution of methyl 3-(1H-pyrrol-2- yl)propanoate (800 mg, 5.2 mmol) in anhydrous DMF (2 mL ). The mixture was then heated to 40° C. until completion by TLC analysis (14 h). The reaction was cooled back down to rt and diluted with EtOAc (18 mL ), and treated with 4M aqueous NaOH solution (5 mL ). The phases were separated, and the aqueous layer was extracted with EtOAc (3×18 mL ). The combined organic layers were washed with H₂O (5×30 mL ), brine (2×30 mL ) and dried over Na₂SO₄. The resulting solution was concentrated under reduced pressure, to afford the desired aldehyde as a brown oil (630 mg, 66%). The product was used without further purification.

¹H NMR (CDCl₃, 400 MHz) δ: 10.01 (1H, brs), 9.42 (1H, s), 6.89 (1H, appt, J=4.0 Hz), 6.1-6.0 (1H, appt, J=4.0 Hz), 3.73 (3H, s), 3.02 (2H, t, J=7.0 Hz), 2.72 (2H, t, J=7.1 Hz). ¹³C NMR (CDCl₃, 100 MHz) δ: 178.4, 173.3, 116.8, 132.3, 122.1, 109.6, 52.0, 33.3, 22.8. HRMS (ESI) calculated for C₉H₁₁NO₃ [M+H]⁺: m/z 182.0739, found m/z 182.0828. IR umax (film)/cm⁻¹: 3248, 2924, 2853, 1735, 1647.

### Methyl 3-[3-(3-azidopropyl)-4, 4-difluoro-4-bora-3a,4a-diaza-s-indacene-5-yl]propanoate

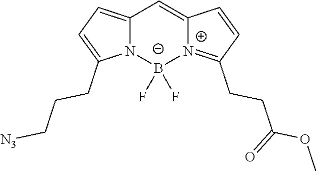 [SMILES: COC(=O)CCC1=[N+]2C(=Cc3ccc(CCCN=[N+]=[N-])n3[B-]2(F)F)C=C1]

A 0° C. solution of 2-(3-azidopropyl)-1H-pyrrole (100 mg, 0.7 mmol) in CH₂Cl₂ (5 mL ), was treated with a solution of 3-(5-formyl-1H-pyrrol-2-yl)propanoate (60 mg, 0.3 mmol) in CH₂Cl₂ (5 mL ). The resulting mixture was then treated by the dropwise addition of POCl₃ (100 μL, 1.0 mmol), and was then stirred at rt for 6.5 h before being cooled back down to 0° C. The reaction mixture was then treated sequentially with BF₃.Et₂O (0.2 mL , 1.6 mmol) followed by N,N-diisopropylethylamine (0.3 mL , 1.7 mmol). The reaction was stirred at rt until completion by TLC analysis (18 h). The reaction mixture was then quenched with H₂O (10 mL ), diluted with CH₂Cl₂ (5 mL ) and filtered through a bed of Celite®. The Celite® was washed with CH₂Cl₂ (2×10 mL ), and the organic phases combined. The combined organic washes were dried over Na₂SO₄, and concentrated under reduced pressure. Purification of the crude residue by flash column chromatography (silica gel, elution gradient 0-30% EtOAc in petroleum ether) yielded the expected ester as a red oil (31 mg, 26%).

¹H NMR (CDCl₃, 400 MHz) δ: 7.15 (1H, s), 7.04 (1H, d, J=4.0 Hz), 7.01 (1H, d, J=4.4 Hz), 6.3 (2H, appt, J=4.0 Hz), 3.72 (3H, s), 3.42 (2H, t, J=6.8 Hz), 3.34 (2H, t, J=7.6 Hz), 3.09 (2H, t, J=7.6 Hz), 2.81 (2H, t, J=7.6 Hz), 2.09-2.02 (2H, m). ¹³C NMR (CDCl₃, 100 MHz) δ: 164.6, 159.9, 156.5, 146.8, 140.1, 134.9, 128.3, 127.9, 120.9, 114.7, 51.8, 50.8, 34.7, 28.0, 25.4, 21.4. ¹⁹F NMR (CDCl₃, 376 MHz) δ: −143.7, −143.8, −143.9, −144.0. HRMS (ESI) calculated for C₁₆H₁₈BF₂N₅O₂ [M−H]⁺: m/z 360.1522, found m/z 360.3250. IR uₘₐₓ (film)/cm⁻¹: 3171, 2936, 2098, 1734, 1609, 1497, 1175.

λ_{exc}=467 nm (at 510 nm emission, c=0.2 nM, MeOH).

λₑₘᵢₛ=509 nm (at 315 nm excitation, c=0.2 nM, MeOH).

### 3-[3-(3-Azidopropyl)-4,4-difluoro-4-bora-3a,4a-diaza-s-indacene-5-yl]propionic Acid

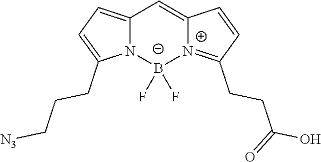 [SMILES: [N-]=[N+]=NCCCc1ccc2n1[B-](F)(F)[N+]1=C(CCC(=O)O)C=CC1=C2]

A 0° C. solution of methyl 3-[3-(3-azidopropyl)-4,4-difluoro-4-bora-3a,4a-diaza-s-indacene-5-yl]propanoate (30 mg, 80 μmol) in THF (4 mL ) was treated with H₂O (2.6 mL ), followed by concentrated HCl (1.6 mL ). The reaction mixture was then stirred at rt until completion by TLC analysis (18 h). The reaction mixture was then diluted with CH₂Cl₂ (20 mL), and stirred at rt for 1 h. The organic layer was separated, and washed with H₂O (2×20 mL) followed by brine (2×25 mL). The resulting solution was dried over Na₂SO₄, and concentrated under reduced pressure to afford the expected carboxylic acid as a red oil (15 mg, 52%). The product was used without further purification.

¹H NMR (CDCl₃, 400 MHz) δ: 7.16 (1H, s), 7.05 (1H, d, J=4.0 Hz), 7.01 (1H, d, J=3.2 Hz), 6.39-6.38 (2H, m), 3.42 (2H, t, J=6.8 Hz), 3.35 (2H, t, J=7.6 Hz), 3.10 (2H, t, J=7.6 Hz), 2.86 (2H, t, J=7.6 Hz), 2.10-2.02 (2H, m). ¹³C NMR (CDCl₃, 100 MHz) δ: 166.2, 161.6, 159.9, 153.1, 149.0, 134.9, 130.8, 128.8, 127.9, 118.5, 50.9, 31.9, 30.5, 26.0, 23.8. ¹⁹F NMR (CDCl₃, 376 MHz) δ: −143.75, −143.84, −143.92, −144.01. HRMS (ESI) calculated for C₁₅H₁₆BF₂N₅O₂ [M−H]⁺: m/z 346.1365, found m/z 346.3293. IR uₘₐₓ (film)/cm⁻¹: 3423, 2915, 2850, 1644.

λ_{exc}=467 nm (at 510 nm emission, c=5 nM, MeOH)

λₑₘᵢₛ=510 nm (at 315 nm excitation, c=5 nM, MeOH).

### 4″-O—[3-(5-(3-Azidopropyl)-4,4-difluoro-4-bora-3a,4a-diaza-s-indacene-3-yl)propanoate]-5-O-(tert-butyldimethylsilyl)avermectin B1a

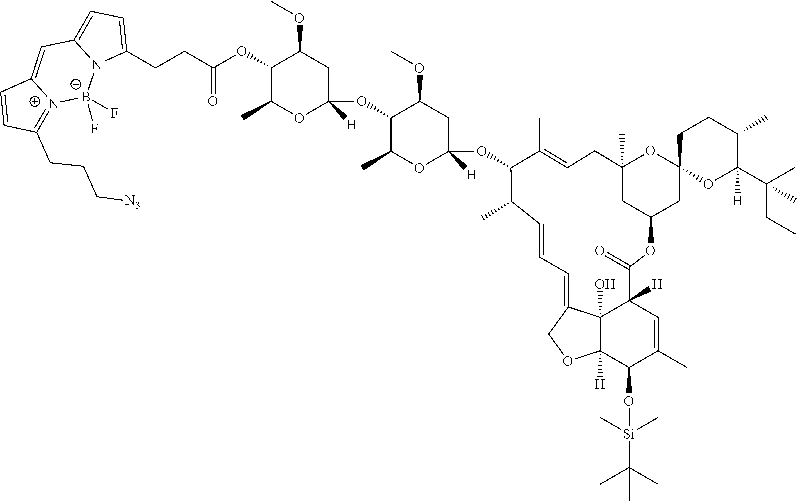

A solution of 5-O-(tert-butyldimethylsilyl)avermectin B1a (40 mg, 40 μmol) in CH₂Cl₂ (2.5 mL ) was treated sequentially with a solution of 3-(5-(3-azidopropyl)-4,4-difluoro-4-bora-3a,4a-diaza-s-indacene-3-yl)propanoic acid (15 mg, 40 μmol) in CH₂Cl₂ (2.5 mL), followed by DCC (16 mg, 70 μmol) and DMAP (3 mg, 20 μmol). The reaction mixture was then stirred at rt until completion by TLC analysis (18 h). The reaction was then concentrated under reduced pressure and the crude residue was purified by flash column chromatography (silica gel, elution gradient 0-20% EtOAc in petroleum ether) to afford 4″-O—[3-(5-(3-azidopropyl)-4,4-difluoro-4-bora-3a,4a-diaza-s-indacene-3-yl)propanoate]-5-O-(tert-butyldimethylsilyl)avermectin B1a as a red oil (23 mg, 39%).

¹H NMR (CDCl₃, 400 MHz) δ: 7.14 (1H, s), 7.00 (2H, d, J=4.4 Hz), 6.43 (1H, d, J=4.0 Hz,), 6.37 (1H, d, J=4.0 Hz), 5.85-5.82 (1H, m), 5.77-5.72 (2H, m), 5.41 (1H, br s,), 5.36-5.31 (2H, m), 5.00 (1H, d, J=9.6 Hz), 4.79 (1H, brs), 4.74 (1H, appt, J=10.0 Hz), 4.71-4.68 (1H, m), 4.60 (1H, d, J=14.4 Hz), 4.45 (1H, brs), 3.96 (1H, brs), 3.88-3.83 (3H, m), 3.70-3.60 (3H, m), 3.45 (6H, br s), 3.41-3.37 (4H, m), 3.34-3.31 (1H, m), 3.28-3.20 (2H, m), 2.86-2.79 (4H, m), 2.53 (1H, brs), 2.38-2.32 (2H, m), 2.29 (1H, appd, J=11.6 Hz), 2.24 (1H, dd, J=12.0, 4.0 Hz), 2.00 (1H, dd, J=12.8, 4.4 Hz), 1.98-1.93 (2H, m), 1.81 (3H, s), 1.79-1.62 (9H, m), 1.53 (3H, s), 1.41-1.36 (3H, m), 1.29-1.27 (6H, brs), 1.17 (1H, d, J=6.8 Hz), 0.96-0.92 (12H, m), 0.89-0.86 (4H, m), 0.81-0.79 (1H, m), 0.15 (6H, s). ¹³C NMR (CDCl₃, 100 MHz) δ: 174.0, 172.7, 162.1, 160.5, 140.2, 137.5 (2C), 135.0 (2C), 134.8, 130.4, 127.9, 124.8, 119.3, 118.7, 118.4, 118.3, 117.2, 98.5, 97.5, 94.8, 81.9, 80.6, 80.4, 80.2, 80.0, 79.3, 79.2, 77.2, 76.5, 69.5, 68.7, 67.9, 67.2, 67.1, 56.8, 56.5, 51.8, 45.7, 41.1, 39.6, 36.8, 35.7, 35.4, 34.9, 34.5, 34.1, 33.2, 33.0, 31.2, 29.7, 28.1, 27.3, 25.8, 25.4, 20.3, 20.0, 18.4, 17.7, 17.4, 15.2, 12.4, 12.1, −4.5, −4.8. ¹⁹F NMR (CDCl₃, 376 MHz) δ: −144.21, −144.30, −144.39, −144.48. HRMS (ESI) calculated for C₇₁H₁₀₆BF₂N₅O₁₅Si [M−H]+: m/z 1344.7516, found m/z 1344.3033. IR umax (film)/cm⁻¹: 3360, 2961,2921, 2851, 1632.

### 4″-O—[3-(5-(3-azidopropyl)-4,4-difluoro-4-bora-3a,4a-diaza-s-indacene-3-yl)propanoate]avermectin B1a, 15

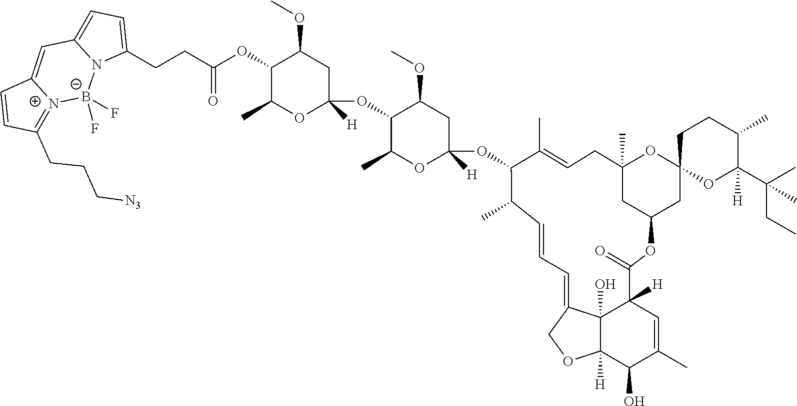

A solution of 4″-O—[3-(5-(3-azidopropyl)-4, 4-difluoro-4-bora-3a,4a-diaza-s-indacene-3-yl)propanoate]-5-O-(tert-butyldimethylsilyl)avermectin B1a (6 mg, 4 μmol) in MeOH (2.0 mL ) was treated with a catalytic amount of p-TsOH (3 mg). The reaction mixture was stirred at 18° C. for 30 min. The reaction was then diluted with H₂O (15 mL ) followed by EtOAc (20 mL ). The organic layer was washed with H₂O (3×20 mL ) and brine (20 mL ). The organic phase was dried over Na₂SO₄ and concentrated under reduced pressure. Purification of the crude residue by flash column chromatography (0-40% EtOAc/petroleum ether) afforded 4″-O—[3-(5-(3-azidopropyl)-4,4-difluoro-4-bora-3a,4a-diaza-s-indacene-3-yl)propanoate]avermectin B1a, 15 as a red oil (4 mg, 72%). MS (ESI) calculated for C₆₅H₉₂BF₂N₅O₁₅ [M−H]⁺: m/z 1230.6651, found m/z 1230.1914.

General Method for Reacting Protein with Conjugate Precursor

To a 10 mL flask were added maleimide compound (12 mmol), iLOV protein (10 mg, 0.7 mmol) and 1 mL of pH7 phosphate buffer, with stirring at room temperature for 1 h. After this time the reaction mixture was added to a column loaded with 1 mL of Strep-Tactin Superflow resin (IBA Lifesciences) and 1 mL of pH 8 buffer (150 mM TRIS, 100 mM NaCl). The column was sealed and incubated on a rotor for 15 min at 4° C. Unreacted compounds were eluted with pH 8 buffer (4 mL ) before addition of SUMO protein (15 μL) and pH 8 buffer (100 mM TRIS, 150 mM NaCl). The column was sealed and incubated on the rotor for 18 h at 4° C. After the incubation, solution (now green in color) was collected in tubes before concentration in centrifuge at 14,000 rpm for 7 min. The concentration was calculated by Bradford protein assay and the conjugate solutions were stored at −80° C. until required. Compounds P1 to P11 were generated in this way (see Table 1 below).

### Conjugate Uptake Studies

Uptake studies using conjugates of the invention were carried out on a number of organisms, focusing particularly on uptake by C. elegans.

The conjugates tested are shown in the table below, together with reference compounds also tested for comparison.

TABLE 1
Conjugates Tested

| Compound | Structure |
|---|---|
| 1 | 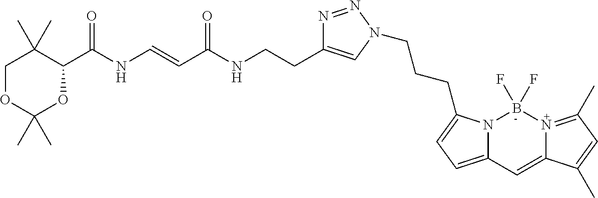 |
| 2 | 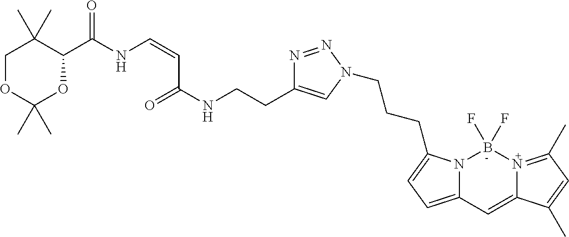 |
| 3 | 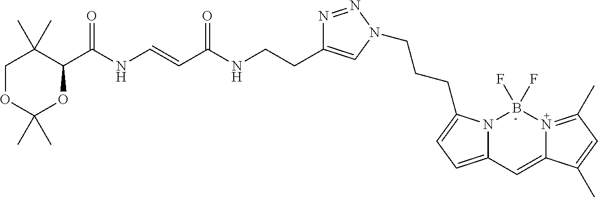 |
| 4 | 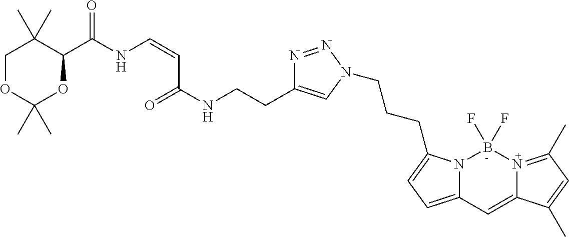 |
| 5 | 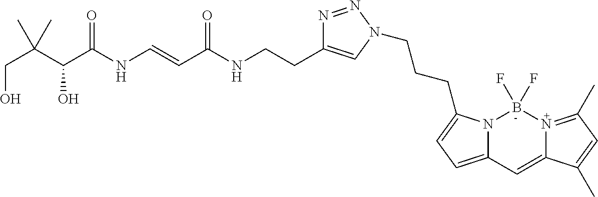 |
| 6 | 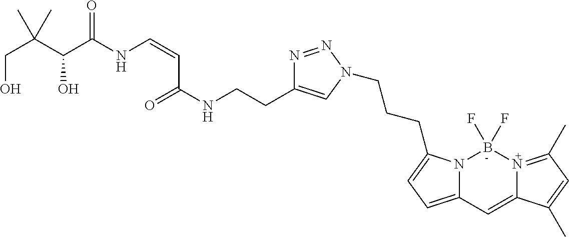 |
| 7 | 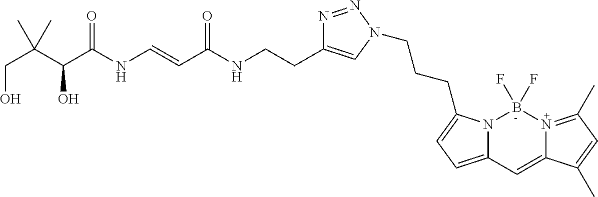 |
| 8 | 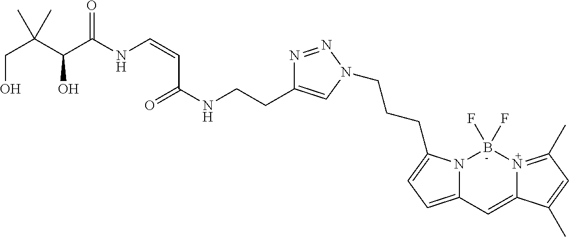 |
| 9 | 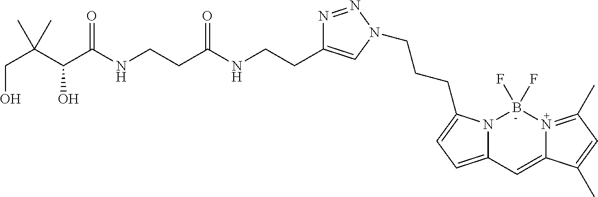 |
| 10 | 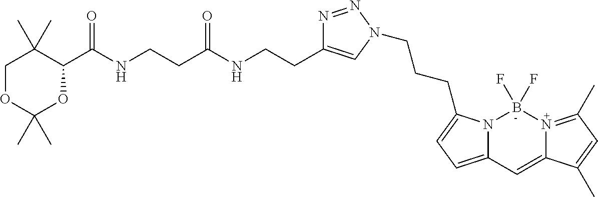 |
| 11 | 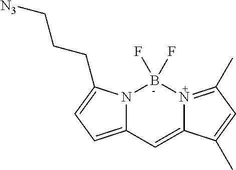 |
| 12 | 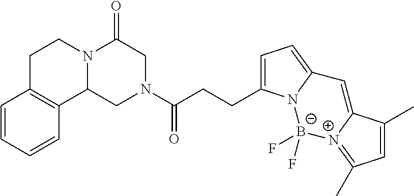 [SMILES: CC1=CC(C)=[N+]2C1=Cc1ccc(CCC(=O)N3CC(=O)N4CCc5ccccc5C4C3)n1[B-]2(F)F] |
| 13 | 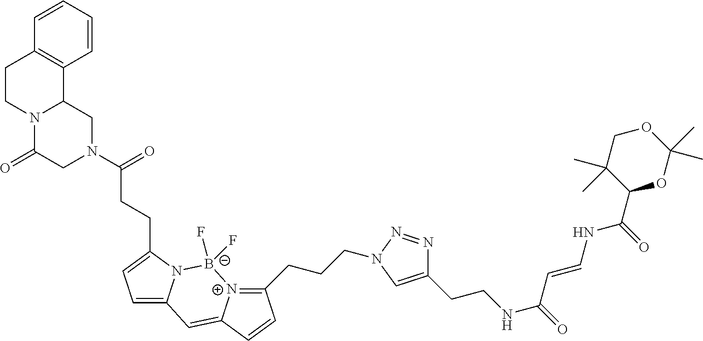 [SMILES: CC1(C)OCC(C)(C)[C@H](C(=O)N/C=C/C(=O)NCCc2cn(CCCC3=[N+]4C(=Cc5ccc(CCC(=O)N6CC(=O)N7CCc8ccccc8C7C6)n5[B-]4(F)F)C=C3)nn2)O1] |
| 14 | 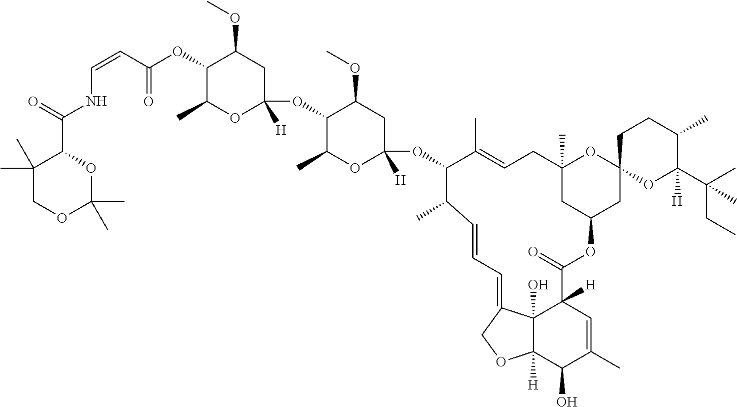 |
| 15 | 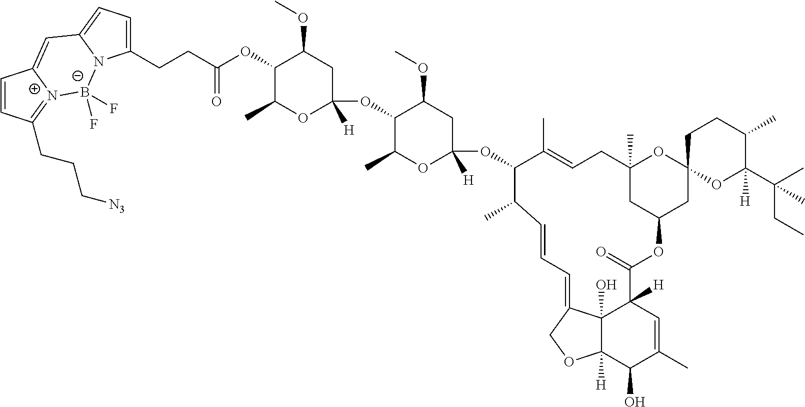 |
| P1 | 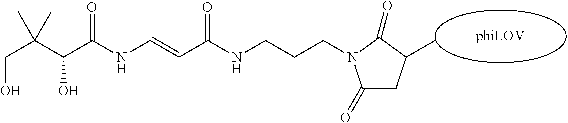 [SMILES: CC1CC(=O)N(CCCNC(=O)/C=C/NC(=O)[C@H](O)C(C)(C)CO)C1=O] |
| P2 | 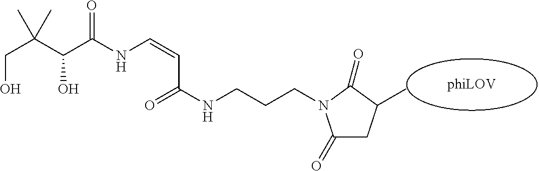 [SMILES: CC1CC(=O)N(CCCNC(=O)/C=C\NC(=O)[C@H](O)C(C)(C)CO)C1=O] |
| P3 | 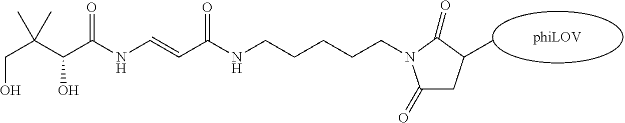 [SMILES: CC1CC(=O)N(CCCCCNC(=O)/C=C/NC(=O)[C@H](O)C(C)(C)CO)C1=O] |
| P4 | 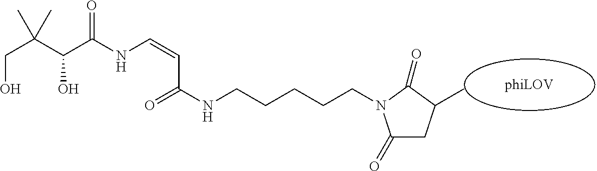 [SMILES: CC1CC(=O)N(CCCCCNC(=O)/C=C\NC(=O)[C@H](O)C(C)(C)CO)C1=O] |
| P5 | 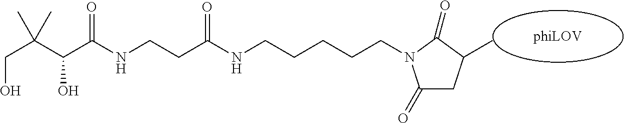 [SMILES: CC1CC(=O)N(CCCCCNC(=O)CCNC(=O)[C@H](O)C(C)(C)CO)C1=O] |
| P6 | 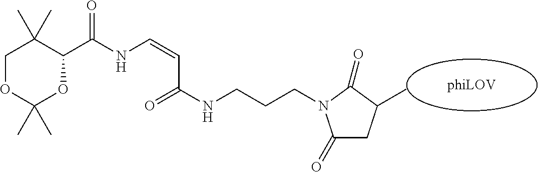 [SMILES: CC1CC(=O)N(CCCNC(=O)/C=C\NC(=O)[C@@H]2OC(C)(C)OCC2(C)C)C1=O] |
| P7 | 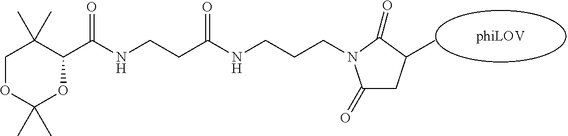 [SMILES: CC1CC(=O)N(CCCNC(=O)CCNC(=O)[C@@H]2OC(C)(C)OCC2(C)C)C1=O] |
| P8 | 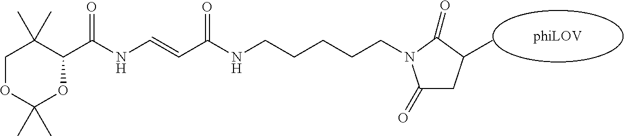 [SMILES: CC1CC(=O)N(CCCCCNC(=O)/C=C/NC(=O)[C@@H]2OC(C)(C)OCC2(C)C)C1=O] |
| P9 | 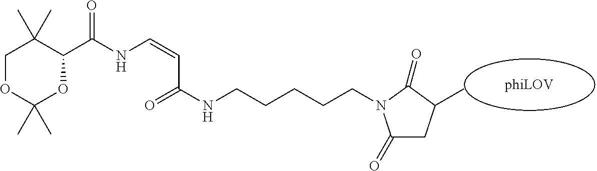 [SMILES: CC1CC(=O)N(CCCCCNC(=O)/C=C\NC(=O)[C@@H]2OC(C)(C)OCC2(C)C)C1=O] |
| P10 | 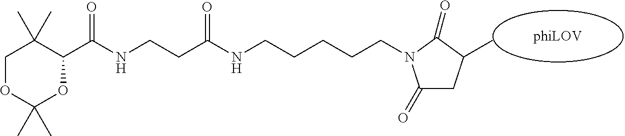 [SMILES: CC1CC(=O)N(CCCCCNC(=O)CCNC(=O)[C@@H]2OC(C)(C)OCC2(C)C)C1=O] |
| P11 | 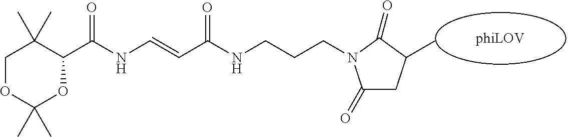 [SMILES: CC1CC(=O)N(CCCNC(=O)/C=C/NC(=O)[C@@H]2OC(C)(C)OCC2(C)C)C1=O] |

C. elegans Testing

Nematode Growth Media (NGM) plates containing C. elegans were washed with 1.0-1.5 mL of standard M9 buffer and the washes were collected in an Eppendorf tube. The liquid containing the C. elegans was then spun for 1 min at room temperature at 10,000 rpm. The supernatant was removed, and fresh M9 buffer was added (500 μL) to generate a new stock solution of M9 buffer containing the C. elegans.

A C. elegans suspension (100 μL) was added a defined amount of M9 buffer (determined by the final desired concentration of compound to be tested: 399 μL in the case of the BODIPY tagged derivatives) contained within a separate Eppendorf tube. To this was then added a 25 mM solution of the compound in DMSO (1 μL). The resulting suspension was mixed using a vortex instrument. Once the mixing was complete, the Eppendorf tube was placed on its side to ensure that the worms did not settle to the bottom of the tube, and the test compound as evenly dispersed.

The resulting mixture was incubated at room temperature for 2 to 4 h. in the dark (using aluminium foil to screen the tube, which was also stored in a cabinet).

Other samples were incubated overnight. Here, OP50 bacteria (3 μL) were added to the worms for feeding purposes, and the worms were kept in the dark at 20° C.

After the incubation, the tube contents were mixed gently by hand (typically by simple rotation of the tube), so as to achieve a homogenous suspension. A sample of the suspension (250 μL) was taken and mixed with M9 buffer (500 μL). The resulting suspension was placed in a centrifuge and spun at room temperature at 10,000 rpm for 1 min. The supernatant was removed, and the washing process was repeated twice more, each time using further quantities of fresh M9 buffer (500 μL each time). After the final washing sufficient supernatant was removed so as to leave a final sample volume of approximate 30 μL.

A 1.5% agarose solution in water was pre-warmed for 60 to 90 s. in a standard household style microwave (set to Power 4). Two drops of this solution was removed and placed on a glass slide. The glass slide was covered with a second glass slide. After 1 min., the slides were slid carefully apart, with the agarose remaining on one of the slides. Onto this slide was placed an aliquot of the final C. elegans solution (15 to 20 μL). To this was added a 20 mM sodium azide solution (5 μL) to inhibit nematode mobility. Vaseline was then placed around the agarose area before replacing the second slide. The slides were not pressed together.

The nematodes were then analysed using a Zeiss Axiskop 2 Plus microscope with a Hamamatsu Orca-ER (CA 742-95) camera. The images were resolved using OpenLab on a Mac operating system.

The uptake studies demonstrate that uptake into C. elegans may be optimised, and the location of accumulation may be altered, by changes to the structure of the conjugate. This is shown by way of example using the compounds described below.

Dye Uptake in C. elegans

Compounds 1-11 were evaluated after 3 h and overnight incubation at a 50 μM concentration using the general uptake protocol described for C. elegans.

### Compound 1—(R)-t-kCJ-BODIPY

After 3 h incubation, compound 1 could be observed in the pharynx, gut lumen and gut cells of both adults and larval stages. This was the largest uptake observed compared to compounds 2 to 8 after the 3 h incubation period. After incubation for 20 h, strong fluorescence was localised in the pharynx, gut lumen and gut cells of both adults and larvae.

### Compound 2—(R)-c-kCJ BODIPY

After 3 h incubation, traces of compound 2 were observed in the pharynx, gut cells and gut lumen of the larval stages. No adults were present on the slide. After incubation for 20 h, fluorescence was localised in the gut cells, gut lumen and to a lesser extent within the pharynx of the larvae.

### Compound 3—(S)-t-kCJ BODIPY

After 3 h incubation, compound 3 was present in the pharynx, gut cells and gut lumen of the larval stages. The uptake was to a much lower degree than the enantiomeric analogue compound 1 consistent with an active uptake. In the adults, the fluorescence was detected in the pharynx, gut cells and gut lumen. Fluorescence was also present in the early stage of egg development. After overnight incubation, fluorescence was visible within the pharynx, gut cells and gut lumen of larvae.

### Compound 4—(S)-c-kCJ BODIPY

After 3 h incubation, traces of compound 4 were present in the gut cells of larval stages. After incubation for 20 h, fluorescence was detected in the pharynx and the gut cells of the larval stages as well as in the early stage of egg development.

### Compound 5—(R)-t-dCJ BODIPY

After 3 h incubation, small amounts of compound 5 were present in the gut cells, and the gut lumen of the larval stages, as well as in the pharynx, gut lumen and gut cells of adult nematodes. Compound 5 was not detected in the embryos. After incubation for 20 h, fluorescence remained in the pharynx, gut cells and gut lumen of both the larval and adult stages. Fluorescence was also detected in the early stage of egg development, but not in the embryos.

### Compound 6—(R)-c-dCJ BODIPY

After 3 h incubation, compound 6 was visible in the gut cells and gut lumen of larval stages and in a lesser extent in the pharynx. In adults, fluorescence was located in the pharynx, gut lumen and gut cells. Fluorescence was not observed in the embryos. The uptake was to a much lower degree than the enantiomeric analogue compound 8 consistent with an active uptake. After incubation for 20 h, fluorescence was present in the gut lumen and gut cells, and to a smaller degree in the pharynx of the larvae. Fluorescence was also present in the pharynx, gut cells and gut lumen of the adults as well as in the early stages of egg development. Fluorescence was not observed in the embryos.

### Compound 7—(S)-t-dCJ BODIPY

After 3 h incubation, small amounts of compound 7 were visible in the gut cells and gut lumen, but not in the pharynx of the larvae. In the adults, fluorescence was located in the gut lumen and gut cells, and to a lesser extent in the pharynx. After incubation for 20 h, fluorescence was present in the gut cells, gut lumen but not in the pharynx of the larval stages. Fluorescence was also detected in the gut lumen, gut cells and to a lesser extent in the pharynx of the adults. Fluorescence was also located in the early stages of egg development.

### Compound 8—(S)-c-dCJ BODIPY

After 3 h incubation, compound 8 was visible in the gut cells and gut lumen and to a lesser extent into the pharynx of larvae. This was the second largest uptake observed compared to compounds 1 to 7 after the 3 h incubation period. In adults, fluorescence was observed in the pharynx, the gut lumen, the gut cells, and eggs. After incubation for 20 h, fluorescence was present in the pharynx, gut lumen and gut cells of the larvae. In adults, fluorescence was detected in the pharynx, gut lumen and gut cells, but not in the embryos.

### Compound 9—Pantothenic Acid BODIPY

After 3 h incubation, traces of compound 9 were visible in the get cells and to a lesser extent 30 in the pharynx of both the larvae and the adult nematodes. After incubation for 20 h, small amounts of fluorescence could be detected in the gut cells of both larval and adult stages. In both cases, the amount of fluorescence was lower than the enamide derivatives, compounds 1 to 8.

### Compound 10—Ketal Pantothenic Acid BODIPY

After 3 h incubation, traces of compound 10 were detected in the gut cells and gut lumen of the larvae. Compound 10 was also visible to a lesser degree in the pharynx of the larvae. No adults were present on the slide. After 20 h incubation, fluorescence was visible in the gut cells and in a smaller amount in the pharynx of the larval stages. The amount of fluorescence present after 20 h was comparable with the enamide compounds showing the least uptake.

### Compound 11—BODIPY Azide

Worms exposed to BODIPY 11, showed slight fluorescence after 3 h, focalized in the gut lumen and gut cells, with no significant increase in fluorescence even after 20 h incubation.

Praziquantel Uptake in C. elegans

In order to compare the ability of a conjugate to improve the uptake of the drug praziquantel, two praziquantel derivatives were generated. Praziquantel was tagged with a BODIPY fluorescent tag (compound 12) and incorporated into a conjugate of the invention (compound 13). Compounds 12 and 13 were evaluated at a concentration of 50 μM using the C. elegans uptake protocol described above for compounds 1-11. Microscopic readings were taken after 3, 24 and 72 h.

### Compound 12—Praziquantel BODIPY

After 3 h incubation, compound 12 was weakly up taken up into the pharynx and gut cells of the C. elegans larvae, as well as by adult nematodes mainly in the gut cells with lesser amount in the pharynx. After 24 h incubation, the larva showed no increased uptake, with very minor amounts of compound 12 located in the pharynx and the gut cells. No adult nematodes were present in the 24 h sample taken. After 72 h incubation, only the gut cells of the larvae showed the presence of small amounts of fluorescence. In the case of the adult stages, fluorescence was visible in the gut lumen, gut cells and pharynx. The nematodes remained viable with no apparent detrimental effect.

Compound 13—Conjugate with Praziquantel-BODIPY

After 3 h incubation, compound 13 was strongly located in the pharynx, gut cells and gut lumen of the larval stages. In the adult nematodes, compound 13 was strongly located in the pharynx, gut cells and gut lumen, whilst not visible in the embryos. After 20 h incubation, there was a threefold increase in uptake of compound 13 compared to compound 12 despite its larger molecular weight. After 24 h incubation, compound 13 was strongly observed in the pharynx, gut cells and gut lumen of the larva. No adult nematodes were in the slide at this time point. After 72 h, compound 13 was still strongly visible in the pharynx, gut lumen and get cells of the larvae. In the case of the adult nematodes, compound 13 was strongly visible in the pharynx and in the gut lumen, as well as in the embryos and eggs. The nematodes remained viable with no apparent detrimental effect.

Protein Uptake in C. elegans

Conjugates containing the PhiLOV protein were tested against C. elegans.

The effective PhiLOV protein conjugate in M9 buffer was taken from the chemical ligation and purification experiments. The amount of M9 buffer added was determined by the final concentration required. Nematodes (30-40 per protein sample to be analysed) were picked and added to the protein solution, and the resulting suspension was treated with OP50 E. coli solution for nematode sustenance. The nematode suspension was incubated at 20° C. in the dark, and covered with aluminium foil.

Aliquots (10 to 25 μL) were taken at 24, 48 and 72 h. and transferred into a new Eppendorf tube at each time point and diluted with M9 buffer (200 μL). The new suspension was placed in ice (for 1-2 min) and then centrifuged at 10.000 rpm for 1 min. The supernatant was removed and the concentrated nematodes were washed again (2×200 μL). In the final wash, a residual volume (approx. 30 μL) was left with the nematodes.

For the microscopy studies, a 1.5% agarose solution in water was warmed up for 1-1.5 min in a standard household microwave (Power 4). Two drops of the agarose solution were placed on a glass slide and covered with another glass slide. After 1 min, the two slides were taken apart from each other carefully. On the slide where the agarose remained, 15-20 μL of the final C. elegans solution was placed. To this was added a 20 mM sodium azide solution (5 μL) to inhibit nematode mobility.

Vaseline was placed around the agarose area before replacing the top cover slip. The cover slips were not pressed together. The nematodes were then analysed using a Zeiss Axiskop 2 Plus microscope using a Hamamatsu Orca-ER (CA 742-95) camera. The images were resolved using OpenLab on a Mac operating system.

Feeding was repeated after 48 h. with addition of 2 μL of OP50 bacteria for feeding purposes, and the worm incubation was continued in the dark at 2° C.

Using the approach described above, 11 protein conjugates were tested for their ability to deliver the phiLOV protein into nematodes. The results show that the conjugates are able to deliver large biological molecules to different parts of the nematode. The location and efficiency of the delivery can be modulated by small structural modifications to the delivery vehicle, as shown by compounds P1-P11 (including negative control, positive control using compound 1 and untagged phiLOV protein control).

### Negative Control—M9 Buffer.

After 24 h. incubation with M9 buffer solution only, both the larval and adult stages of C. elegans showed a small amount of cellular autofluorescence in the gut cells. After 48 h. and 72 h., there was no change in the autofluorescence levels. At the 72 h point, the adult nematodes were dead.

### Positive Control—Compound 1—(R)-t-kCJ BODIPY

After 24 h. incubation with compound 1 fluorescence could be observed in the pharynx, gut lumen and gut cells of both adults and larval stages of C. elegans. After 48 and 72 h. incubation fluorescence was still localised in the pharynx, gut lumen and gut cells of both adults and larvae. At the 72 h point, the adult nematodes were dead.

Protein Control—phiLOV Protein

After 24 h. incubation with untagged phiLOV protein (13 kDa protein used at a concentration of 236 μM), a small amount of fluorescence was visible in the pharynx of the adult stages, whilst no fluorescence was present in the larval stages. After 48 and 72 h., there was no change in the larval stages (i.e. no fluorescence was visible). The small amounts of fluorescence in the pharynx of the adult stages also showed no discernible change.

### Compound P1

After 24 h incubation with protein conjugate P1 (305 μM), only autofluorescence of the gut cells within the larval stages could be detected. In adults, a small amount of protein was located in the pharynx. After 48 h incubation with protein conjugate P1, fluorescence could be observed in the pharynx and gut cells of the larval stages. A smaller amount of fluorescence was also visible in the gut lumen. No adults were on the slide. After 72 h, the fluorescence levels remained unchanged.

### Compound P2

After 24 h. incubation with protein conjugate P2 (305 μM) fluorescence was observed in the pharynx as well as the gut lumen of the larval stages. A smaller amount of fluorescence was also visible in the gut cells. Protein conjugate P2 showed no significant improvement in the uptake relative to the untagged protein control. No adult stages were present on the slide. After 48 h. incubation, the larval stages showed increased amounts of fluorescence in the pharynx, gut cells and gut lumen. The adult stages showed localisation of fluorescence in the pharynx and in the gut lumen. After 72 h., the fluorescence remained localised in the pharynx, gut lumen and gut cells of the larvae. In adults, fluorescence was observed in the pharynx and in the gut lumen.

### Compound P3

After 24 h. incubation with protein conjugate P3 (305 μM) small amounts of fluorescence were visible in the pharynx and gut lumen of the larval stages. In the adult stages, fluorescence was visible in the pharynx, gut lumen and gut cells. After 48 h. incubation, significant increase in the amount of fluorescence was detected in the pharynx, gut cells and to a lesser extent in the gut lumen of the larvae. In the adults, increased fluorescence was seen in the pharynx and the gut lumen. After 72 h. incubation, fluorescence was visible in the pharynx, gut lumen and gut cells of the older larval stages. The adult stages showed fluorescence in the gut lumen and gut cells.

### Compound P4

After 24 h. incubation with protein conjugate P4 (305 μM), fluorescence was visible in the pharynx, gut lumen and to a lesser extent, the gut cells of the larvae. In the adults, fluorescence was observed in the pharynx, gut lumen and gut cells. Protein conjugate P4 showed no significant improvement in the uptake relative to the untagged protein control. After 48 h. incubation, fluorescence was present in the pharynx and gut lumen of the larvae. In the adults, the fluorescence was present in the pharynx and the gut lumen. After 72 h. incubation, there was a very large amount of fluorescence present in the pharynx and gut lumen, with a smaller amount of fluorescence in the gut cells of the larval stages. In the adults, fluorescence was clearly visible in the gut cells, gut lumen and pharynx.

### Compound P5

After 24 h. incubation with protein conjugate P5 (305 μM), fluorescence was present in the gut lumen and gut cells of the larvae. In the adults, fluorescence could be observed in the gut lumen and to a lesser extent in the pharynx. After 48 h. incubation, fluorescence could be seen in the gut cells and gut lumen and to a lower amount in the pharynx of the larvae. No adults were present on the slide. After 72 h., fluorescence could be observed in the gut lumen and gut cells of the larvae in a variable amount. No protein could be detected in the pharynx of the larvae. No adults were present on the slide.

### Compound P6

After 24 h. incubation with protein conjugate P6 (305 μM), only endogenous autofluorescence could be seen either the adult or the larval stages. After 48 h. incubation, fluorescence was present in the gut lumen and gut cells as well as in the pharynx of the younger larvae. In the adults, fluorescence was observed in the gut lumen. After 72 h., fluorescence was present in the gut lumen of the larval stages. No adults were on the slide.

At higher concentrations of protein conjugate P6 (435 μM) low fluorescence was seen in the pharynx and in the gut lumen of larvae after 24 h. Only endogenous autofluorescence could be seen in adults after 24 h. After 48 h., the larvae still displayed low amounts fluorescence in the pharynx, gut lumen and gut cells. No adults were on the slide. After 72 h., the larvae showed small amounts of fluorescence in the gut cells and gut lumen. No adults were present on the slide.

### Compound P7

After 24 h. incubation with protein conjugate P7 (305 μM), fluorescence was present in the gut lumen of the larvae. In the case of the adult stages, fluorescence was present in the gut cells and to a lesser extent within the pharynx. After 48 h. incubation, fluorescence could be observed in the gut lumen and gut cells of the larvae as well as in the pharynx of the younger stages of the larvae. No adults were present on the slide. After 72 h. incubation, fluorescence was visible in the gut lumen and gut cells of the larval stages. No adults were present on the slide.

At higher concentrations of protein conjugate P7 (435 μM), fluorescence was visible in the gut lumen and to a lesser extent in the pharynx of the larvae after 24 h. incubation. In the adults, fluorescence was detected in the gut lumen and in smaller amounts in the pharynx after 24 h. After 48 h. incubation, the larvae showed high levels of fluorescence in the gut lumen and to a lesser extent in the pharynx. No adults were present on the slide. After 72 h., high levels of fluorescence were in the gut lumen of the larval stages. Smaller amounts of fluorescence were visible in the pharynx of larvae. No adults were present on the slide.

### Compound P8

After 24 h. incubation with protein conjugate P8 (305 μM), small amounts of fluorescence were observed in the gut cells of the larvae. Small amounts of fluorescence were also present in the pharynx of the adults. Protein conjugate P8 showed significantly reduced uptake relative to protein conjugate P11. After 48 h, small amounts of fluorescence could be detected in the gut lumen and gut cells of the larval stages. No adults were present on the slide. After 72 h., small levels of fluorescence remained in the gut lumen and gut cells of the larval stages. No adults were present on the slide.

At higher concentrations of protein conjugate P8 (435 μM), fluorescence was visible in the gut lumen and to a lower extent into the pharynx of the larvae after 24 h. After 24 h. incubation, fluorescence was observed in the gut lumen and to a lesser extent in the pharynx of the adults. Protein conjugate P8 showed significantly reduced uptake relative to protein conjugate P11. After 48 h. incubation, fluorescence was visible in the gut lumen of the larvae with variable amounts in the gut cells and low distribution in the pharynx. In the adults, fluorescence could be seen in the pharynx and the gut lumen. After 72 h., the larvae showed the presence of fluorescence in the gut lumen and to a lesser extent in the pharynx. No adults were present on the slide.

### Compound P9

After 24 h. incubation with protein conjugate P9 (305 μM) small amounts of fluorescence were visible in the gut lumen of the larvae and in the pharynx of the adults. After 48 h. incubation, fluorescence could be observed in the gut lumen and gut cells of both the larval and adult stages. After 72 h. incubation, fluorescence was present in the gut lumen and gut cells in young larvae. Fluorescence could also be seen in the pharynx, gut cells and gut lumen of adults.

After 24 h. incubation at high concentrations of the protein conjugate P9 (435 μM), fluorescence could be seen in the gut lumen of the larvae. No adults were present on the slide. After 48 h. incubation, increased fluorescence was observed in the gut lumen on the larval stages. No adults were present on the slide. After 72 h. incubation, the larvae showed protein uptake in the gut lumen with smaller amounts in the pharynx and gut cells. No adults were present on the slide.

### Compound P10

After 24 h. incubation with protein conjugate P10 (305 μM), fluorescence was present in the gut lumen of larvae. Low fluorescence was also detected in the adults' pharynx. After 48 h., the larvae showed a higher level of fluorescence in the gut lumen and gut cells. Fluorescence was also visible in the adults' gut lumen. After 72 h., fluorescence was visible in the gut cells, gut lumen and to a lesser extent in the pharynx of the larvae. No adults were on the slide.

At high concentrations of the protein conjugate P10 (435 μM), fluorescence could be seen in the gut cells and gut lumen of the larval stages after 24 h. In the case of the adults, low fluorescence was visible in the pharynx. After 48 h. incubation, the larvae showed fluorescence in the gut cells and in the gut lumen, with a lesser amount in the pharynx. In the adults, fluorescence was visible in the gut lumen. After 72 h., fluorescence was present in the gut cells, gut lumen and pharynx of the larvae. No adults were on the slide.

### Compound P11

After 24 h. incubation with protein conjugate P11 (267 μM), fluorescence was visible in the gut lumen and to a lesser extent in the pharynx of the larvae. Protein conjugate P11 showed significant improvement in the uptake relative to the untagged protein control and compound P8. This supports the hypothesis that the protein conjugate P11 is being actively transported and not just ingested by the worm. In the adult stages, fluorescence was observed in the pharynx and in smaller amounts in the gut lumen. After 48 h. incubation, the larvae showed fluorescence in the gut lumen and gut cells, with lesser amounts in the pharynx. Fluorescence in the adults was located in the gut lumen, and in smaller amounts in the pharynx. After 72 h., the larval stages showed fluorescence in the gut cells, gut lumen and in smaller amounts in the pharynx. No adults were present on the slide.

Plasmodium falciparum Testing

P. falciparum were cultured using a complete medium of RPMI 1640 (Thermo Fisher) containing 10% Albumax II serum (Thermo Fisher). The compounds used for testing were prepared as 100 mM solution in DMSO and diluted to the appropriate concentrations using complete medium. For the testing a 96 well plate with an appropriate number of wells, each containing 50 μL of blood with 200 μL of serum, were used. The parasitemia range was approximately 3-5%. For each well the serum was removed and replaced with an equal volume of serum with the compound to be tested. The 96 well plate was then incubated at 37° C. under an atmosphere of 96% N2, 3% CO₂ and 1% O₂ for 45 minutes. After this the cells were concentrated via centrifugation at 2,209 rpm. The medium was removed and the cells resuspended with another 200 μL of fresh serum. The cells were centrifuged and washed in this manner twice more. After the final washing about 5-10 μL of blood was placed on a slide and a coverslip placed on top smearing the blood. The edges of the slide were then sealed with nail varnish to ensure the cells were contained before imaging. The slides were analysed using a Zeiss Axioplan 2 microscope system and images were taken using Volocity 3D Image Analysis software with a FITC fluorescent filter.

Compounds 1, 5, 9, 10 and 11 were tested for uptake in both synchronised stage and mix-stage cultures at 25 μM concentrations. As expected, compound 11 diffused into every red blood cell (both infected and non-infected) through what appears to be a diffusion controlled mechanism. Compound 1 was not taken up either by infected nor non-infected red blood cells in every culture. Compound 5, however, was selectively taken up by infected red blood cells, and the fluorescent compound was accumulated within the parasite itself (i.e. the compound was internalised into the parasite within the infected erythrocyte). Compound 10 was not taken up to any visible extent, while compound 9 was taken up very quickly by both infected and non-infected erythrocytes, with very little difference in uptake between the two sets of red blood cells.

Additional Plasmodium falciparum Testing

The BODIPY analogue—Amide Conjugate 310—shown below was partially purified via HPLC.

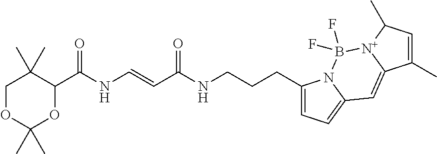

The compound was then incubated at a concentration of 25 μM with a 50 μL aliquot of trophozoite-infected erythrocytes (trophozoite stage P. falciparum) for 45 to 60 minutes.

Trophozoite stage parasites gave the best results when imaging under the fluorescent microscope due to their size. After incubation, the sample was centrifuged and the supernatant removed. The cells were washed with appropriate buffer to remove background fluorescence resulting from the compound in solution. A slide was then prepared from the washed, live cells, and examined using fluorescence microscopy on an Applied Precision DV Elite microscopy system. An inverted microscope and an oil-immersion 100× objective lens were used. Images were taken using a CoolSNAP_HQ2/HQ2-ICX285 camera and acquired using softWoRx version 5.5.

The images show that the compound is accumulated by the parasite within the infected erythrocyte. For healthy erythrocytes there is an absence of fluorescence, and so there is selectivity between healthy and infected cells, possibly due to uptake through the new permeation pathways (NPP) created when a healthy cell becomes infected (see Saliba et al. and Kirk et al.). The fluorescence is absent from the digestive vacuole, but present throughout the cytosol of the parasite.

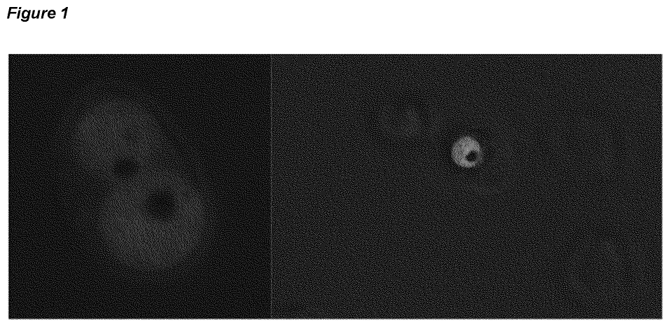

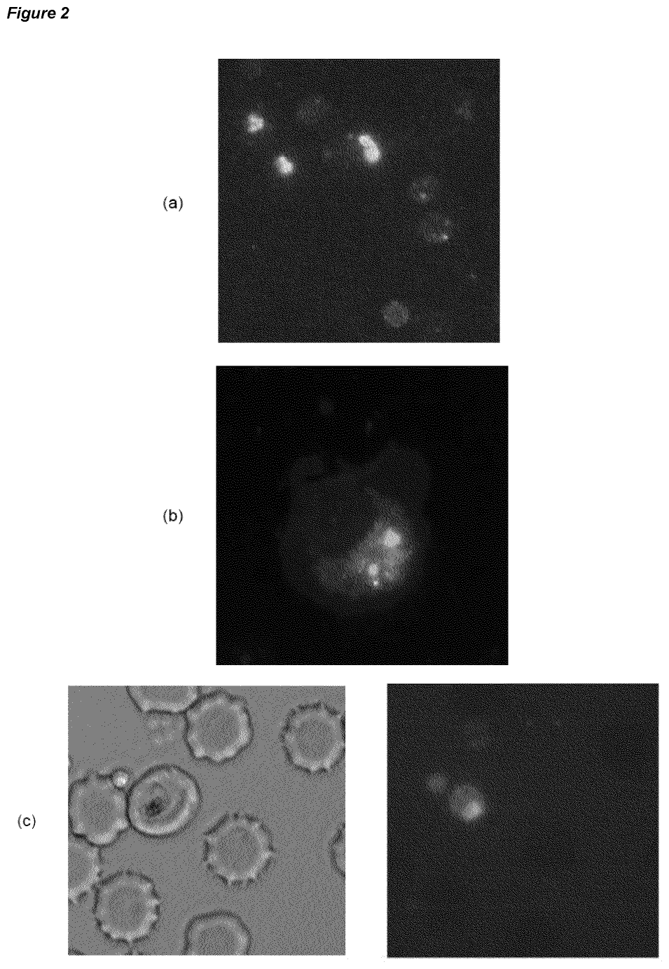

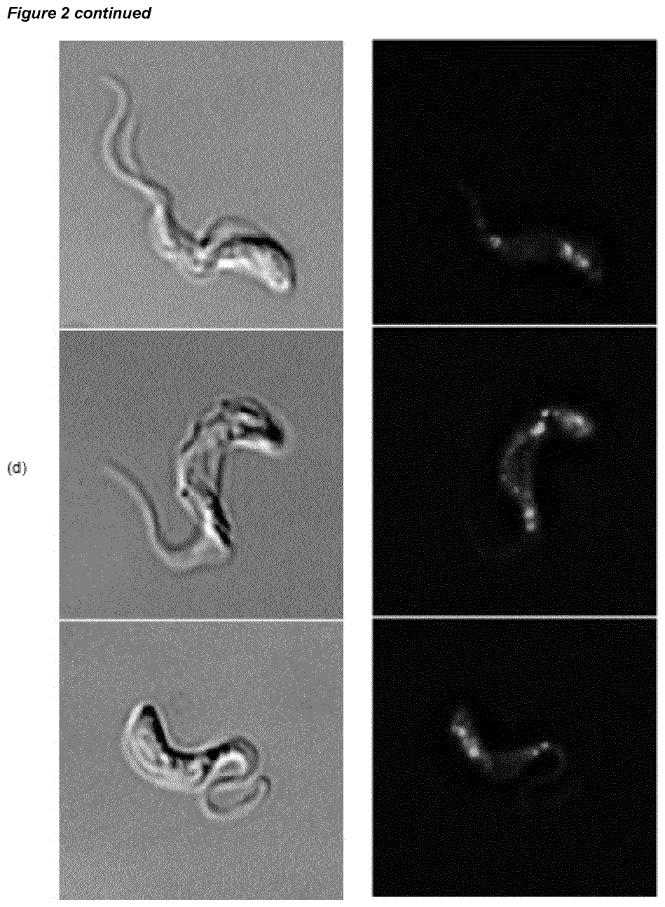

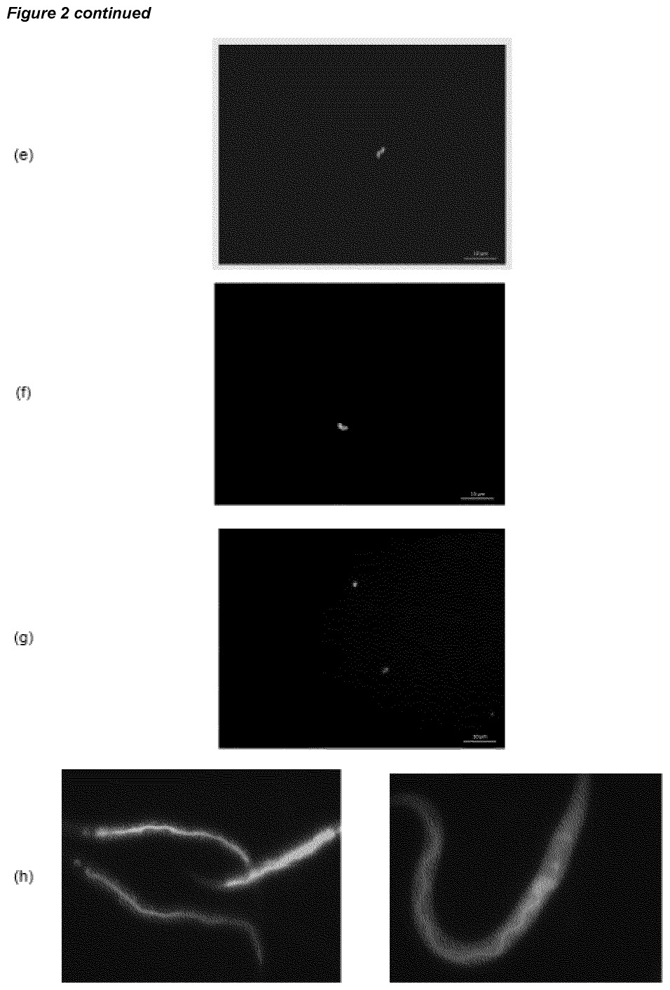

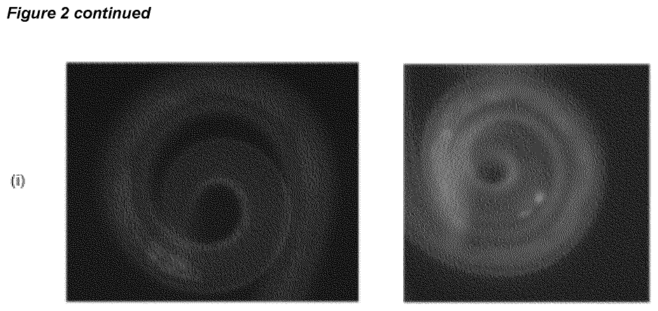

Within the cytosol we can also see spots of high fluorescence are observable, showing areas of high conjugate concentration. Examples are shown in FIG. 1.

Trypanosoma brucei Testing

T. brucei were cultured using a complete medium of RPMI 1640 (Thermo-Fisher) containing 10% Albumax II (Thermo-Fisher) serum. The compounds for testing were prepared as 100 mM solution in DMSO and diluted to the appropriate concentrations using complete medium. For the testing, a 96 well plate with an appropriate number of wells, each containing trypanosomes with 200 μL of serum, were used. For each well, the serum was removed and replaced with an equal volume of serum with the compound to be tested (10-25 μM). The 96 well plate was then incubated at 37° C. under an atmosphere of 96% N₂, 3% CO₂ and 1% O₂ for 45 minutes. After this time the trypanosomes were concentrated via centrifugation at 2,209 rpm. The medium was removed and the cells re-suspended with another 200 μL of fresh serum. The trypanosomes were centrifuged and washed in this manner twice more. After the final washing about 5-10 of serum was placed on a slide and a coverslip placed on top. The edges of the slide were then sealed with nail varnish to ensure the cells were contained before imaging. The slides were analysed using a Deltavision deconvolving microscope system.

The uptake of compounds 5 and 9 in T. brucei was studied.

The images suggest that compound 5 is taken up by T. brucei and forms small vesicles all over the trypanosome body, whilst not accumulating neither in the lysosome nor in the nucleus, but rather in a vesicle between the flagellar pocket and the lysosome.

Compound 9 on the other hand, is taken up much more rapidly and in much higher concentrations than compound 5. Compound 9 is also spread throughout the cell, but it does seem to have areas of higher concentration either in the mitochondrion or in the endoplasmic reticulum.

Theileria annulata Testing

T. annulata were cultured using RPMI 1640 with 10% Albumax II (Thermo-Fisher) serum. A standard hemocytometer was used to calculate the concentration of cells in the parent culture. The culture was diluted with RPMI 1640 to a concentration of 2×10⁵ cells per mL. For each compound and concentration to be tested, 2 mL of the culture were centrifuged at 1,000 rpm for 5 minutes. The medium was removed before the cells were resuspended in 950 μL of complete medium containing the compound to be tested at the desired concentration. This was prepared from a 100 mM stock solution of compound in DMSO. Once the cells were resuspended they were transferred to a 5 well plate (1 mL capacity per well). They were then incubated for 1 hour at 37° C. in an atmosphere of 5% CO₂ in air. After the incubations were complete the contents of each well were transferred to a 10 mL centrifuge tube. Cold RPMI 1640 (Thermofisher) or HBSS added to obtain a volume of 5 mL. The suspension was then centrifuged at 1,000 rpm and at 4° C. for 5 minutes. The supernatant was then removed and the cells were washed in this manner twice more. After the final washing the pellet of cells was resuspended in a minimum amount of RPMI 1640 and then spotted onto a glass slide. The slide was then covered with a cover slip and analysed using Olympus BX60 UV microscope system with a Spot RT3 camera and Spot Image software with a FITC fluorescent filter.

The uptake of compounds 1, 5, 9, 10, 11 in T. annulata was studied. The images suggest that compounds 5 and 9 are taken up by T. annulata. Compound 9 is taken up more rapidly and fluorescence can be detected at lower concentrations albeit it is also present in the infected lymphocyte. Compound 5 on the other hand, is selectively accumulated within the intracellular parasite. Compound 11 diffused indiscriminately, while compounds 1 and 10 showed very limited uptake.

### Bacteria Testing

Bacteria cultured by inoculating LB media (Thermo-Fisher) with the desired bacteria and incubating overnight at 37° C. under an atmosphere of air. The OD₆₀₀ was then measured and the concentration of bacteria calculated from this. The cultures were then diluted appropriately to give a concentration of 2×10⁵ cells per mL. 1 mL of this culture was placed in a 5 mL centrifuge tube then centrifuged at 13,000 rpm for 5 minutes. The supernatant was removed and replaced with 1 mL of 25 μM solution of compound in media. The cultures were incubated for 45 minutes at 37° C. The cultures were centrifuged as before, the supernatant removed and the pellet resuspended in 1 mL of PBS. The cells were washed in this manner twice more. After the final washing the pellet was resuspended in 35 μL of water and spotted onto a slide. The slide was then protected with a coverslide before the images were analysed using an Olympus UV microscope system with a Leica imaging analysis software with a FITC fluorescent filter.

In the case of the bacterial cultures, compound 5 was tested in every case. Uptake was seen both in the case of Gram-negative and Gram-positive bacteria.

### Cytotoxicity

The conjugates of the invention are based on pantothenate and CJ-15,801. The former is an essential vitamin, and is tolerated in vivo in human subjects.

The cytotoxicity of the conjugates may be measures against HEK cells using the method described below.

A sample compound was incubated at 100 μM with human embryonic kidney cells (HEK293) in Greiner 384 cell star plates for 24 hours before adding CellTitre-Glo® (Promega), an indicator of cell viability giving a luminescent output. In this assay, compounds showing greater than 40% cytotoxicity over two test runs at 100 μM are deemed to have shown cytotoxicity.

Interestingly, the representative compound shown below was inactive in the screen, 3.7±0.6% at the 100 μM concentration.

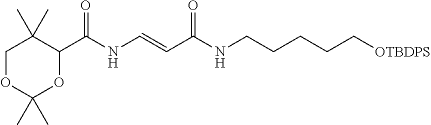

The compound above may be prepared as shown below, with hexafluorophosphate benzotriazole tetramethyl uranium (HBTU), N,N-Diisopropylethylamine (DIPEA), CH₂Cl₂, μW 80° C. for 2.5 hr. The yield was 58%.

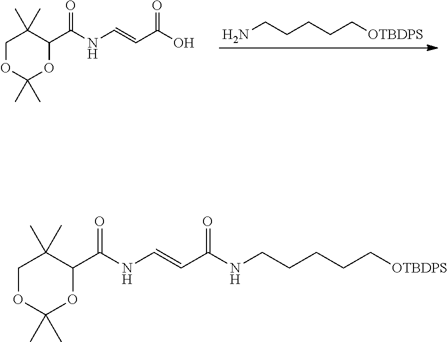

The parent carrier was tested for intrinsic clearance in female CD 1 mouse microsomes, exhibiting a clearance of 1.0 mL/min/g of liver. Here, the carrier is a CJ-15,801 framework with an amide linkage.

Lotmaria passim Testing

In the last years, bee colony collapse disorder represents one of the key issues for honey bee's survival. European honey bees (Apis mellifera) have played a significant role in agriculture, and organism-model to identify pathogens and symbionts. Belonging to Kinetoplastea class, Lotmaria passim (L. passim) has emerged as the predominant parasite in honey bees worldwide (Schwarz, et al., Ravoet et al., and Paxton et al.).

### Parasite Uptake Test (CJ-15,801 and Pantothenic Acid Derivatives Coupled to BODIPY FL).

100 mM stock solutions of BODIPY derivatives were prepared with dimethylsulfoxide (DMSO). L passim strain PRA-403 (ATCC) was cultured in modified medium for C. bombi (Tognazzo et al.). The parasite culture was centrifuged, supernatant was removed and 500 μL of L passim medium with compound (final concentration 500, 100, 10 or 1 μM for BODIPY derivatives) or DMSO (0.1%) control was added. Incubated for 45 min at room temperature. After the incubation, the samples were centrifuged and pellet washed with PBS (3×500 μL), samples were transferred to slide and be observed by fluorescent microscope (NIKON ECLIPSE Ni) or added 100 μL to transfer to 96-wells microplate and fluorescence intensities were measured using a Varioskan LUX or BioTek microplate reader.

### Honey Bee Gut Uptake Test (CJ-15,801 and Pantothenic Acid Derivatives Coupled to BODIPY FL).

Honey bee (A. mellifera) hives were obtained and allowed to feed ab libitum spring to autumn. During the winter period, pollen mixed with 50% (v/v) sucrose was added to the honey bee hives in order to provide sufficient food. Honey bees were dissected and gastrointestinal system was removed including the crop, midgut and rectum, and added immediately to 500 μL of the delivery vehicle to be tested sample diluted with PBS at 100 μM final concentration or DMSO (0.1%) control. The mixture was incubated for 45 min at 33° C. After the incubation, the samples were centrifuged and guts washed with PBS buffer (3×500 μL), samples were transferred to slides and observed by fluorescent microscope (NIKON ECLIPSE Ni).

### Results—Parasite Uptake Test

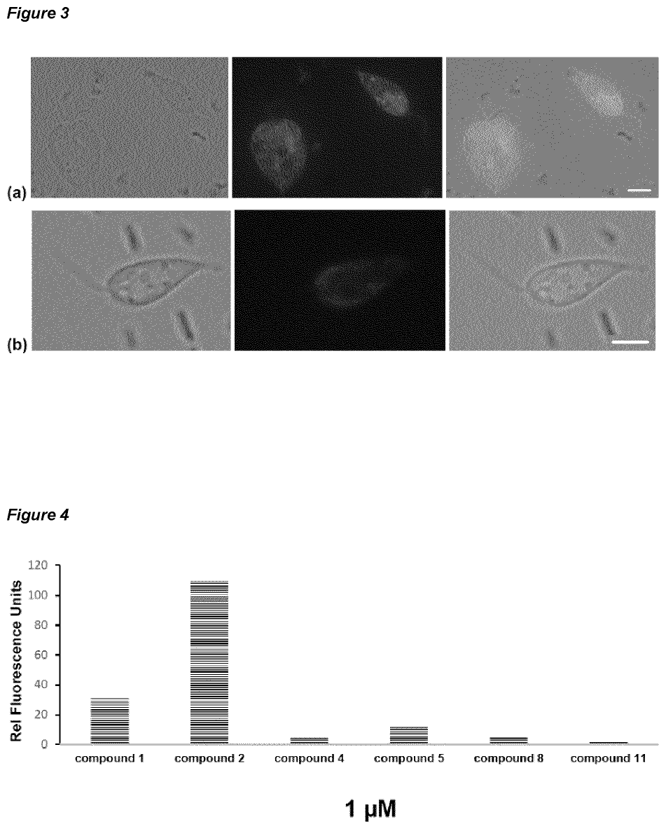

A set of CJ-BODIPY derivatives were tested in L. passim strain PRA-403 at 10 and 500 μM, incubated at room temperature for 45 min. After incubation, the samples were analysed under fluorescence microscopy. Parasites treated with (R)-trans-ketal 2 at 500 μM exhibited fluorescence in the parasite's cytosol, with the derivative forming fluorescence dots. On the other hand, parasites incubated with the diol derivative (S)-c/s-diol 8 at 10 μM displayed fluorescence surrounding the cell membrane but not in the cytoplasm (FIG. 3).

Uptake tests were performed at 1 μM and 100 μM final concentration of CJ-BODIPY derivatives. After incubation at room temperature for 45 min, fluorescence intensities were evaluated using a microplate reader. The quantitative results confirmed the fluorescence microscope findings, which suggested the preferential uptake of the ketal derivatives. Derivative 2 showed the highest reading amongst all the compounds tested, whilst derivative 4 showed one of the lowest amounts of uptake. Compound 1 showed approximately % of the fluorescence of derivative 2 at 1 μM. Compounds 5 and 8 had relatively low uptake (FIG. 4).

### Results—Gut Uptake Test

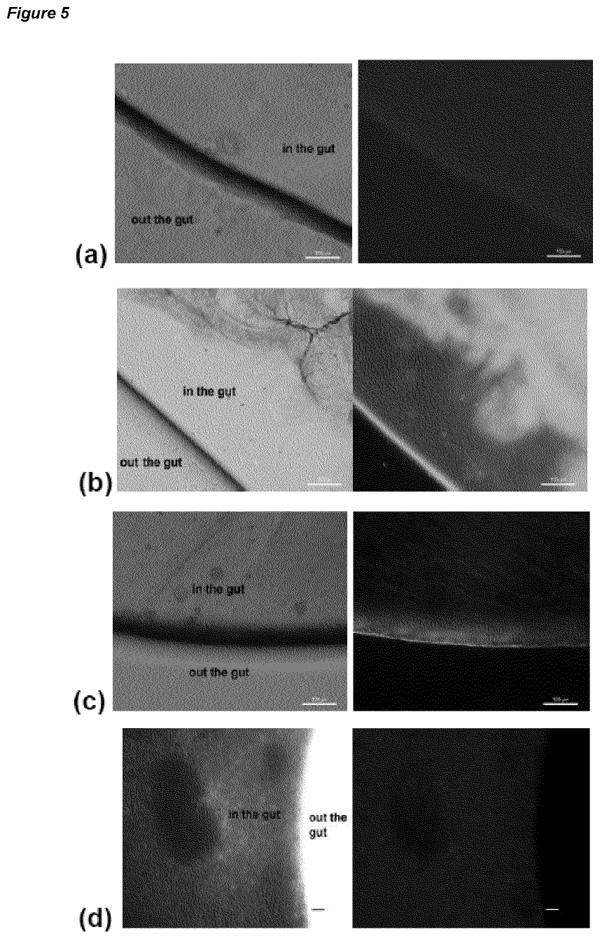

The gastrointestinal system (crop, midgut and rectum) of adult honey bees was extracted, and incubated with derivatives 1, 2, 8 and 11 at 100 μM and 33° C. for 45 min. Derivatives 2 8 displayed slight fluorescence in midgut and rectum, but none in the crop. Compound 2 is localised on the surface of the gut, but not inside of it. Guts treated with BODIPY 11 showed only autofluorescence. Incubation with derivative 1 resulted in high fluorescence in the midgut and rectum, but not the crop (FIG. 5).

Without wishing to be bound by theory, the results suggest that using derivatives at concentrations below 100 μM would diminish the probability of distributing the tested compounds outside of the bees' gastrointestinal system, and would maintain the compounds close to the parasite.

Thus, these derivatives appear to be suitable tools for the delivery of molecules into Lotmaria passim, with minimal permeability through honey bees' gastrointestinal system.

Babesia bovis. Testing

B. bovis is a protozoan parasite, which shares the taxonomic group with Theileria, and is placed in the Piroplasmida order due to its pear-shaped appearance within the infected erythrocytes. Babesia reproduces by binary fission, causing the characteristic presence of pairs or tetrads in stained infected erythrocytes. Two species, B. bovis and B. bigemina, cause considerable economic impact in the cattle industry (Yamagishi et al.)

Short-Term Test in B. bovis (CJ-15,801 and Pantothenic Acid Derivatives Coupled to BODIPY FL and phiLOV(R475G, K476C)).

100 mM stock solutions of the delivery vehicles were prepared with DMSO. B. bovis Texas strain (sourced from The University of Nagasaki) was cultured in GIT medium (Kohjin-bio Co) with 10% Bovine red blood cells (RBC) by a microaerophilic stationary-phase culture system (Bork et al.). The parasite culture was centrifuged and washed once with GIT medium. RBC's were pellet down, and 20 μl infected RBC mixture (10 μl packed infected RBC+10 μl GIT) was prepared. Then, 180 μl GIT medium with compound the compound to be evaluated (final concentration 25 μM for BODIPY derivatives and 100 μM for phiLOV derivatives) or DMSO (0.1%)/PBS control was added. The samples were incubated for 45 min at 37° C. (Samples were stained 1 μg/mL diluted Hoechst 33342 in GIT medium). After incubation, the samples were centrifuged and the pellet was washed with GIT medium (3×). A final solution of 50% haematocrit infected RBC mixture was transferred to slides to be observed by confocal laser microscopy (Nikon A1). The level of parasitemia was monitored by staining thin blood smears with Giemsa solution.

FACS Experiment in B. bovis (CJ-15,801 and Pantothenic Acid Derivatives Coupled to phiLOV(R475G, K476C)).

B. bovis Texas strain was cultured in GIT medium (Kohjin-bio Co) with 10% Bovine RBC by a microaerophilic stationary-phase culture system (Bork et al.). The parasite culture was centrifuged and washed once with GIT medium. RBC's were pelleted down and 20 μl infected RBC mixture (10 μl packed with infected RBC+10 μl GIT) was prepared. Then, 180 μl GIT medium with the test compound (final concentration 150-250 μM) or DMSO (0.1%)/PBS control was added. The samples were incubated for 45 min at 37° C. (Samples were stained using DRAQ5 at a 20 μM final concentration in GIT medium). After the incubation, the samples were centrifuged and the pellet was washed with GIT medium (3×), and transferred to FACS tubes to be analysed by BD FACSVerse.

Long-Term Test in B. bovis (CJ-15,801 and Pantothenic Acid Derivatives Coupled to phiLOV(R475G, K476C)).

B. bovis Texas strain was cultured in GIT medium with 10% Bovine RBC by a microaerophilic stationary-phase culture system (Bork et al.). The parasite culture was centrifuged, and washed once with GIT medium. RBC's were pelleted down, and a 20 μL infected RBC mixture (10 μl packed infected RBC+10 μl GIT) was prepared. Then, 180 μL of a GIT medium with the test compounds (final concentration 100 or 5 μM for phiLOV derivatives) or DMSO (0.1%)/PBS control was added. The mixtures were incubated for 45 min at 37° C. After the incubation, the samples were centrifuged and the pellet washed with GIT medium (3×). 180 μL of GIT were then added and the sample incubated at 37° C. The samples were stained with 200× diluted Hoechst 33342 (Thermofisher) in GIT medium and 50% haematocrit infected RBC mixtures were transferred onto a slide to be observed by confocal laser microscopy (Nikon A1) after 5 h and 48 h incubation. At 24 h, fresh GIT media was added to the samples. Parasetimia levels were monitored by staining thin blood smears with Giemsa solution.

### Results

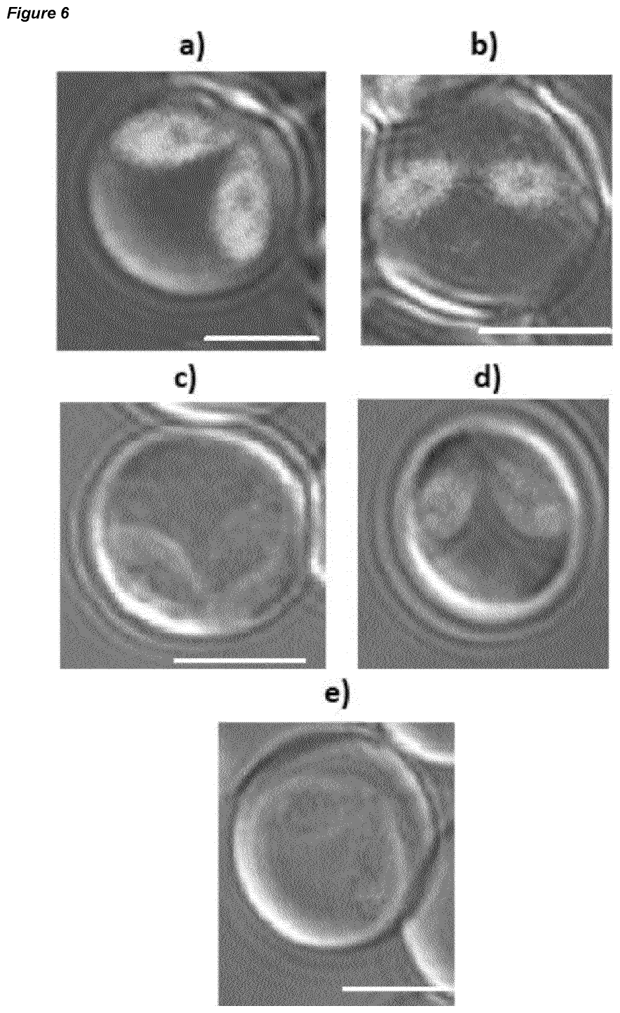

Uptake experiments were performed by incubating the parasite (˜5% Parasitized Erythrocytes (PPE)) with each derivative at 25 μM and 37° C. for 45 min. After incubation, the fluorescence intensities were calculated from confocal microscope images. These images were analysed by ImageJ 1.50b software, following the same procedure applied on C. elegans (Miller et al., and Fricker et al.) (FIG. 6).

Compound 1 exhibited the highest uptake, and was significantly higher than derivative 3 (50% less), Analogue 2 and the untagged BODIPY control 11 (90% less) showed much lower values compared to compound 1. Compound 5 on the other hand, also displayed lower uptake (30% less) compared to the compound 1.

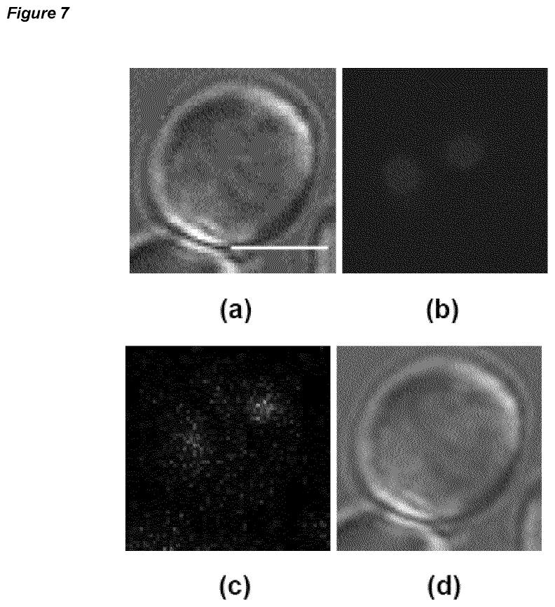

The phiLOV double mutant (R475G, K476C) was used to generate derivatives P11, P1, P8, and P3 which were tested for uptake in B. bovis Texas strain using phiLOV as a control. The derivatives and the control were incubated at 100 μM and 37° C. for 45 min. After incubation, the samples were analysed under confocal microscopy using Hoeschst 33342 (Thermofisher) as co-stain. Only samples treated with derivative P3 showed fluorescence uptake in the characteristic binary form of B. bovis (FIG. 7).

In a separate study, derivatives P11, P1, P8 and P3 as well as untagged phiLOV as a control were incubated at 37° C. for 45 min incubation in ca. 9% Parasitized Erythrocytes (PPE). After incubation, DRAQ5 (Thermofisher) was added as co-stain in order to sort the samples. The FACS results were consistent with the confocal findings, in which derivative P3 exhibited significant uptake and localisation within the parasite.

Parasite growth impact assays were performed by incubating RBCs infected with B. bovis, in the presence of the phiLOV functionalised derivatives in 5 μM and 100 μM for 45 min. The initial PPE was ca. 1% and 3% respectively). Samples were taken at 48 h after exposure, and the PPE was calculated using Giemsa solution before microscopic analysis. All derivatives exhibited similar cell growth, with no significant difference with the control. Parasite reproduction reached comparable levels in all cultures, including those treated with derivative P3, showing the nontoxic of the derivatives on the parasite in long-term experiments.

Mycobacterium testing

Uptake test in M. tuberculosis (CJ-15,801 and pantothenic acid derivatives coupled to BODIPY FL).

20 mg/mL stock solutions of BODIPY derivative compounds 1-8 and 10 were prepared with DMSO. M. tuberculosis strain H37Rv (sourced from the University of Queensland) and centrifuged, media was removed and pellet resuspended with PBS/0.02% tyloxapol, 1 mL aliquots were transferred to Eppendorf tubes, centrifuged, supernatant was removed and pellet was resuspended with 1 mL of BODIPY derivatives (final concentration 50 μg/ml) incubated at room temperature for 45 min, with agitation and protected from light. After the incubation, the samples were centrifuged and pellet washed with PBS (3×100 μL), samples were transferred to 96-well microplate to evaluate the fluorescence intensities in the microplate reader (Agilent). 25 μL were spotted on glass slides before being dried over a heat block at 90° C. for 10 min. Cells were fixed by immersing in 10% formalin for 30 min. Slides were then air dried and mounted with DAKO mounting media (Agilent) before being observed under fluorescence microscopy.

### Results

A set of compounds was tested using M. tuberculosis strain H37Rv. This strain has been studied extensively for biomedical purposes due to the ease of genetic manipulation but more importantly, the cells retain full virulence in animal models for tuberculosis and the susceptibility to drugs.

An uptake experiment was performed by incubating the bacteria with 50 μg/mL (˜80 μM) compounds 1-8, 10 and the control PBS (phosphate-buffered saline) at room temperature for 45 min as described above.

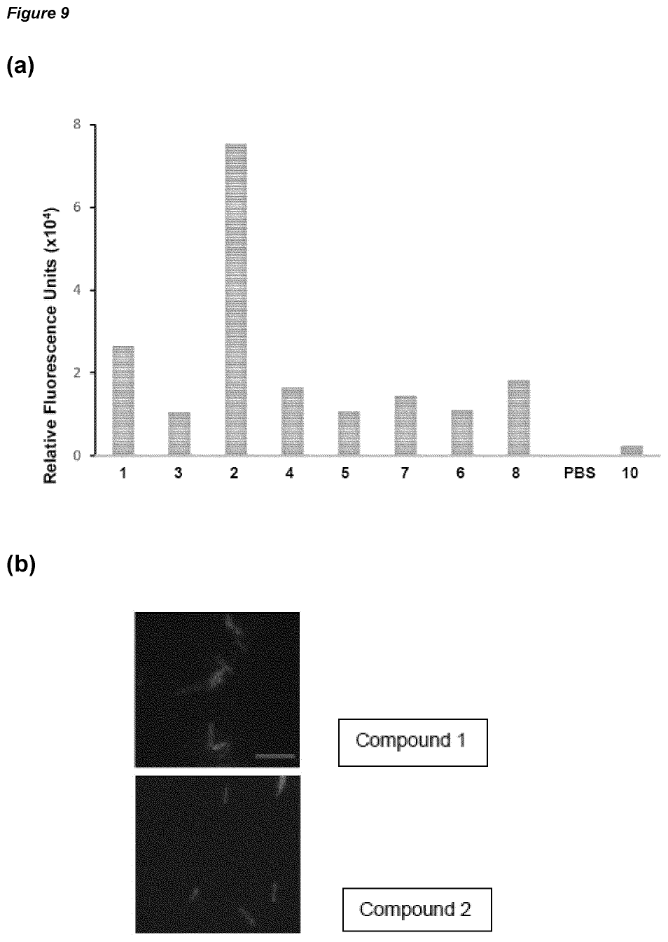

The results are shown in FIG. 9.

The results showed that the enamide ketal derivatives (compounds 1-4) showed higher intensities compared to the enamide diols (compounds 5-8), and that the R enantiomers of the enamide ketal derivatives (compounds 1 and 2) were preferred over their S counterparts (compounds 3 and 4). Compounds 1 and 2 showed the highest fluorescence signals, with compound 2 exhibiting around 2.5 times larger intensity compared to compound 1. Interestingly, the shape of bacteria is clear in the case of compound 2, and fluorescence is localised at the poles, as shown by the images in FIG. 9(b). The pantothenic acid derivative 10 showed minimal uptake.

### Nematode Testing

### Uptake Tests in Nematodes (Ivermectin B1a, Pantothenic Acid Derivative Coupled to Ivermectin and BODIPY-Ivermectin Complex)

A mixed culture of wild type nematodes (roundworms, tape worms and flukes), obtained from Massey University's sheep farm, were incubated for 20 h with compound 14, compound and commercially available ivermectin B1a, as a control. The protocol followed is the same as the C. elegans protocol used previously.

### Results

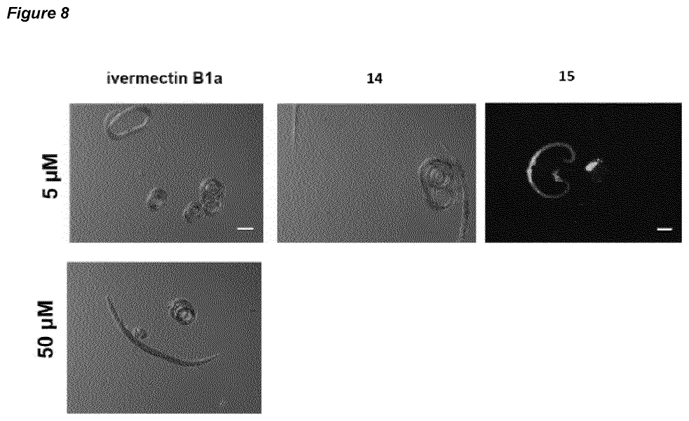

Compound 14 showed enhanced activity against nematodes relative to commercially available ivermectin B1a. Nematodes treated with compound 14 showed paralysis at 5 μM, compared to 50 μM for ivermectin B1a. Compound 15 showed a similar activity profile to that of ivermectin B1a (FIG. 8).

### REFERENCES

EP 0 068 485
U.S. Pat. No. 9,108,942
WO 2012/064632
WO 2012/097454
WO 2019/060634
Akinnusi, T. O. et al., Bioorg. Med. Chem. 2011, 19, 2696.
Aragon et al., Mol. Biochem. Parasitol. 2009, 164, 57.
Bird et al., Am. J. Clin. Nutr. 1955, 3, 298.
Bork, S. et al., Parasitology. 2005, 130 (5), 489.
Clarke, K. M. et al., J. Am. Chem. Soc. 2005, 127, 11234.
Date et al., Advanced Drug Delivery Reviews 2007, 59, 505.
Fricker, G. et al., Pharm Res. 1999, 16 (10), 1570-5.
Gießler et al., Eur. J. Org. Chem. 2010, 2010, 3611.
Green and Wuts; 3rd Edition; John Wiley and Sons, 1999.
Han, C. et al., Org. Lett. 2004, 6, 28.
Kayser et al., Expert Opin. Investig. Drugs 2003, 12, 197.
Kirk, K. et al., Transport and Trafficking in the Malaria infected Erythrocyte, pp 55-66. John Wiley & Sons, Chichester.
Landfear Clinical Pharmacology Therapeutics 2010, 87, 122.
Ma et al., J. Med. Chem. 2004, 47, 6349.
Meier, J. L. et al., J. Am. Chem. Soc. 2006, 128 (37), 12174-12184.
Miller, D. S., Am. J. Physiol. 1995, 269 (2 Pt 2), R370-9.
Paxton, R. J. et al., Journal of Apicultural Research. 2010, 49 (1), 80-84.
Ravoet, J. et al., Journal of Invertebrate Pathology. 2015, 130, 21-27.
Saliba, K. J., J. Biol. Chem. 1998, 273, 10190.
Schwarz, R. S. et al., Journal of Eukaryotic Microbiology. 2015, 62 (5), 567-583;
Sewell, A. L., et al., Org. Lett. 2011, 13, 800.
Shakya, G. et al., J. Am. Chem. Soc. 2014, 136 (48), 16792-16799.
Sparr et al., Antimicrobial Agents Chemotherapy 2013, 57, 4689.
Spry, C. et al., Eur. J. Med. Chem. 2018, 143, 1139.
Storz, et al., J. Am. Chem. Soc. 2012, 134 (39), 16143-16146.
Tognazzo, M. et al., PLoS ONE. 2012, 7(11): e49137.
Van der Westhuyzen et al., Chemistry & Biology 2012, 19, 559.
Woelfle et al., PLoS Negl. Trop. Dis. 2011. 9, 1260.
Yamagishi, J. et al., BMC Genom. 2014, 15 (1), 678.
Zabrodski et al., Synlett, 2015, 26, 461.
Zhao et al., Org. Biomol. Chem. 2010, 8, 3328.

1. A conjugate of formula (I), or a pharmaceutical composition comprising the conjugate of formula (I): 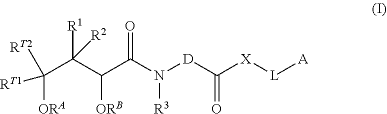
wherein:
—R^{A} and —R^{B} are each independently selected from hydrogen, alkyl, alkenyl, alkynyl, aralkyl, cycloalkylalkyl, alkanoyl and aralkanoyl;
or —R^{A} and —R^{B} together with —C(R^{C1})(R^{C2}) form a 6-membered ring, where
—R^{C1} is independently selected from hydrogen, alkyl, alkenyl, alkynyl, aralkyl and cycloalkylalkyl, and
—R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or
—R^{C1} and —R^{C2} are together oxo (═O);
—R^{T1} and —R^{T2} are each independently hydrogen or alkyl;
—R¹ and —R² are each independently selected from hydrogen, alkyl, alkenyl, alkynyl, aralkyl and cycloalkylalkyl;
—R³ is hydrogen or alkyl;
-D- is C₂₋₄ alkenylene or C₁₋₄ alkylene, where the alkenylene or alkylene is optionally substituted with alkyl or halo;
—X— is a covalent bond, —N(R⁴)—, —O—, —S—, or —Se—, where —R⁴ is hydrogen or alkyl;
-L- is a linker or a covalent bond; and
A- is an active agent for delivery, and comprises a dye, a small drug, a polypeptide, a polynucleotide or a polysaccharide;
and salts, solvates and protected forms thereof.

2. A method for treating a nematode or flatworm infection, comprising contacting the conjugate or the pharmaceutical composition of claim 1 with a nematode or a flatworm.

3. The method of claim 2, wherein the nematode or flatworm is selected from the group consisting of Caenorhabditis nematode, a Haemonchus nematode, and a Schistosoma flatworm.

4. The conjugate or pharmaceutical composition of claim 1, wherein A- is a small drug, a polypeptide, or a polysaccharide.

5-7. (canceled)

8. The conjugate or pharmaceutical composition of claim 1, wherein -D- is C₂ alkenylene or C₂ alkylene, where the alkenylene or alkylene is optionally substituted with alkyl or halo.

9-12. (canceled)

13. The conjugate or pharmaceutical composition of claim 1 wherein —R^{T1} is hydrogen and —R^{T2} is hydrogen or alkyl.

14. The conjugate or pharmaceutical composition of claim 1, wherein —R¹ and —R² are each independently selected from alkyl, alkenyl, aralkyl and cycloalkylalkyl.

15. (canceled)

16. The conjugate of claim 1, wherein —R^{A} and —R^{B} are each independently selected from hydrogen, alkyl, alkenyl, alkynyl, aralkyl, cycloalkylalkyl, alkanoyl and aralkanoyl.

17. The conjugate of claim 1, wherein —R^{A} and —R^{B} together with —C(R^{C1})(R^{C2}) form a 6-membered ring, where —R^{C1} is independently selected from hydrogen, alkyl, alkenyl, alkynyl, aralkyl and cycloalkylalkyl, and —R^{C2} is independently selected from hydrogen, alkyl, alkenyl, alkynyl, aralkyl, cycloalkylalkyl, alkoxy, alkenoxy, alkynoxy, aralkoxy and cycloalkylalkoxy, or —R^{C1} and —R^{C2} are together oxo (═O).

18. (canceled)

19. The conjugate or pharmaceutical composition of claim 1, wherein —R³ is hydrogen.

20. The conjugate or pharmaceutical composition of claim 1, wherein —X— is a covalent bond or —N(R⁴).

21-22. (canceled)

23. The conjugate or pharmaceutical composition of claim 1 wherein -L- is a linker defined by *-L³-B-L⁴-G-L^{A}-,
wherein the asterisk indicates the point of attachment to —X—;
-L³- is a covalent bond, alkylene or heteroalkylene;
-B- is a covalent bond, arylene, heterocyclene, or cycloalkylene;
-L⁴- is a covalent bond, alkylene or heteroalkylene; and
-G- is a covalent bond, —O—, —S—, —N(R^{N})—, —C(O)—, —C(O)N(R^{N})—, —C(O)O—, —N(R^{N})C(O)—, —OC(O)—, and a maleimide-derived group, where —R^{N} is hydrogen or alkyl,
-L^{A}- is a covalent bond, alkylene or heteroalkylene
wherein at least one of -L³-, -B- and -L⁴- is not a covalent bond, and when -B- is a covalent bond, -L⁴- is a covalent bond.

24. The conjugate or pharmaceutical composition of claim 1, wherein -L- is a linker defined by *-L³-B-L⁵-G-,
wherein the asterisk indicates the point of attachment to —X—;
-L³- is a covalent bond, alkylene or heteroalkylene;
-B- is a covalent bond, arylene, heterocyclene, or cycloalkylene;
-L⁵- is an amide group of formula *—(NR^{N}C(O)-L⁶)-, where the asterisk indicates the point of attachment to -B-, and -L⁶- is alkylene;
G- is a covalent bond, —O—, —S—, —N(R^{N})—, —C(O)—, —C(O)N(R^{N})—, —C(O)O—, —N(R^{N})C(O)—, —OC(O)—, and a maleimide-derived group,
and —R^{N} is hydrogen or alkyl.

25-57. (canceled)

58. The method of claim 2, wherein the parasite is selected from a Plasmodium parasite, a Theileria parasite, a Phytophthora parasite, a Babesia parasite, a Crithidia parasite, a Lotmaria parasite, a Trypanosoma parasite, and a Toxoplasma parasite.

59. A method of treating a microbial infection comprising contacting the conjugate or the pharmaceutical composition of claim 1 with a bacterium.

60. (canceled)

61. The method of claim 59, wherein the microbe is selected from a Mycobacteria bacterium, an Escherichia bacterium, a Staphylococcus bacterium, and an Enterococcus bacterium.
